SEQUENCE LISTING (174 sequences)

SEQ ID NO: 1 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
  SITE 7..11: note = Dpr
  SITE 14: note = Dpr
  SITE 22..23: note = Dpr
GXGXFXXXXXXFGXAXVGEXAXX

SEQ ID NO: 2 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
  SITE 7..11: note = Dpr
  SITE 14: note = Dpr
  SITE 22..23: note = Dpr
GXGXFXXXXXXFGXAXVGEXAXXG

SEQ ID NO: 3 - protein, 20 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGEX

SEQ ID NO: 4 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGEXAKK

SEQ ID NO: 5-6 - skipped (2 entries)

SEQ ID NO: 7 - protein, 7 aa, synthetic construct
  SITE 1: note = Dpr
  SITE 4: note = Dpr
  SITE 7: note = Dpr
XFAXFAX

SEQ ID NO: 8 - protein, 14 aa, synthetic construct
  SITE 1: note = Dpr
  SITE 4: note = Dpr
  SITE 7..8: note = Dpr
  SITE 11: note = Dpr
  SITE 14: note = Dpr
XFAXFAXXFAXFAX

SEQ ID NO: 9 - protein, 7 aa, synthetic construct
  SITE 1: note = Dpr
  SITE 4: note = Dpr
  SITE 7: note = Dpr
XYAXYAX

SEQ ID NO: 10 - protein, 14 aa, synthetic construct
  SITE 1: note = Dpr
  SITE 4: note = Dpr
  SITE 7..8: note = Dpr
  SITE 11: note = Dpr
  SITE 14: note = Dpr
XYAXYAXXYAXYAX

SEQ ID NO: 11 - protein, 7 aa, synthetic construct
  SITE 1: note = Dpr
  SITE 4: note = Dpr
  SITE 7: note = Dpr
XFGXFGX

SEQ ID NO: 12 - protein, 14 aa, synthetic construct
  SITE 1: note = Dpr
  SITE 4: note = Dpr
  SITE 7..8: note = Dpr
  SITE 11: note = Dpr
  SITE 14: note = Dpr
XFGXFGXXFGXFGX

SEQ ID NO: 13 - protein, 7 aa, synthetic construct
  SITE 1: note = Dpr
  SITE 4: note = Dpr
  SITE 7: note = Dpr
XFSXFSX

SEQ ID NO: 14 - protein, 14 aa, synthetic construct
  SITE 1: note = Dpr
  SITE 4: note = Dpr
  SITE 7..8: note = Dpr
  SITE 11: note = Dpr
  SITE 14: note = Dpr
XFSXFSXXFSXFSX

SEQ ID NO: 15 - protein, 18 aa, synthetic construct
  SITE 1: note = Dpr
  SITE 4: note = Dpr
  SITE 7..8: note = Dpr
  SITE 11: note = Dpr
  SITE 14..15: note = Dpr
  SITE 18: note = Dpr
XFAXFAXXFAXFAXXFAX

SEQ ID NO: 16 - protein, 18 aa, synthetic construct
  SITE 1: note = Dpr
  SITE 4: note = Dpr
  SITE 7..8: note = Dpr
  SITE 11: note = Dpr
  SITE 14..15: note = Dpr
  SITE 18: note = Dpr
XYAXYAXXYAXYAXXYAX

SEQ ID NO: 17 - protein, 18 aa, synthetic construct
  SITE 1: note = Dpr
  SITE 4: note = Dpr
  SITE 7..8: note = Dpr
  SITE 11: note = Dpr
  SITE 14..15: note = Dpr
  SITE 18: note = Dpr
XFGXFGXXFGXFGXXFGX

SEQ ID NO: 18 - protein, 18 aa, synthetic construct
  SITE 1: note = Dpr
  SITE 4: note = Dpr
  SITE 7..8: note = Dpr
  SITE 11: note = Dpr
  SITE 14..15: note = Dpr
  SITE 18: note = Dpr
XFSXFSXXFSXFSXXFSX

SEQ ID NO: 19 - skipped

SEQ ID NO: 20 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVGEXAKK

SEQ ID NO: 21 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
GXGKFXKKKKKFGKAXVGEXAKK

SEQ ID NO: 22 - protein, 12 aa, synthetic construct
GRKKRRQRRRPQ

SEQ ID NO: 23 - protein, 4 aa, synthetic construct
KLAK

SEQ ID NO: 24 - protein, 14 aa, synthetic construct
KLAKLAKKLAKLAK

SEQ ID NO: 25 - protein, 23 aa, synthetic construct
GIGKFLHSAGKFGKAFVGEIMKS

SEQ ID NO: 26 - protein, 23 aa, synthetic construct
GIGKFLHSAKKFGKAFVGEIMNS

SEQ ID NO: 27 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVGEXAKK

SEQ ID NO: 28 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGSFXHKKKKFGKAXVGEXAKK

SEQ ID NO: 29 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHNKKKFGKAXVGEXAKK

SEQ ID NO: 30 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHQKKKFGKAXVGEXAKK

SEQ ID NO: 31 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHTKKKFGKAXVGEXAKK

SEQ ID NO: 32 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHYKKKFGKAXVGEXAKK

SEQ ID NO: 33 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVWEXAKK

SEQ ID NO: 34 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVVEXAKK

SEQ ID NO: 35 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVLEXAKK

SEQ ID NO: 36 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVYEXAKK

SEQ ID NO: 37 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVFEXAKK

SEQ ID NO: 38 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVTEXAKK

SEQ ID NO: 39 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGDXAKK

SEQ ID NO: 40 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGQXAKK

SEQ ID NO: 41 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGNXAKK

SEQ ID NO: 42 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVVEXAKK

SEQ ID NO: 43 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVFEXAKK

SEQ ID NO: 44 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVVEXAKK

SEQ ID NO: 45 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVFEXAKK

SEQ ID NO: 46 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dbu
  SITE 7..11: note = Dbu
  SITE 14: note = Dbu
  SITE 22..23: note = Dbu
XXGXFXXXXXXFGXAXVGEXAXX

SEQ ID NO: 47 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Orn
  SITE 7..11: note = Orn
  SITE 14: note = Orn
  SITE 22..23: note = Orn
GXGXFXXXXXXFGXAXVGEXAXX

SEQ ID NO: 48 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGE

SEQ ID NO: 49 - protein, 21 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXK

SEQ ID NO: 50 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 5: note = D-phenylalanine
  SITE 12: note = D-phenylalanine
GXGKFXHKKKKFGKAXVVEXAKK

SEQ ID NO: 51 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 5: note = L-4-fluoro phenylalanine
  SITE 12: note = L-4-fluoro phenylalanine
GXGKFXHKKKKFGKAXVVEXAKK

SEQ ID NO: 52 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 5: note = L-4-methyl phenylalanine
  SITE 12: note = L-4-methyl phenylalanine
GXGKFXHKKKKFGKAXVVEXAKK

SEQ ID NO: 53 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 5: note = L-4-trifluoromethyl phenylalanine
  SITE 12: note = L-4-trifluoromethyl phenylalanine
GXGKFXHKKKKFGKAXVVEXAKK

SEQ ID NO: 54 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 5: note = L-4-t-butyl phenylalanine
  SITE 12: note = L-4-t-butyl phenylalanine
GXGKFXHKKKKFGKAXVVEXAKK

SEQ ID NO: 55 - protein, 26 aa, synthetic construct
  VARIANT 2: note = X can be any natural or artificial amino acid
  VARIANT 6: note = X can be any natural or artificial amino acid
  SITE 16: note = X can be any natural or artificial amino acid
  SITE 16: note = X can be any natural or artificial amino acid
  SITE 20: note = X can be any natural or artificial amino acid
GXGKFXKKKKKFGKAXVVEXAKKGGE

SEQ ID NO: 56 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVFEXAKKGGE

SEQ ID NO: 57 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 18: note = D-Phenylalanine
GXGKFXKKKKKFGKAXVFEXAKKGGE

SEQ ID NO: 58 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 18: note = L-4-trifluoromethyl phenylalanine
GXGKFXKKKKKFGKAXVFEXAKKGGE

SEQ ID NO: 59 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 18: note = L-4-t-butyl phenylalanine
GXGKFXKKKKKFGKAXVFEXAKKGGE

SEQ ID NO: 60 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dbu
GXGXFXKKKKKFGKAXVGEXAKK

SEQ ID NO: 61 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 8: note = Dbu
GXGKFXKXKKKFGKAXVGEXAKK

SEQ ID NO: 62 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 14: note = Dbu
GXGKFXKKKKKFGXAXVGEXAKK

SEQ ID NO: 63 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 9: note = Dpr
GXGKFXKKXKKFGKAXVGEXAKK

SEQ ID NO: 64 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dbu
GXGKFXXKKKKFGKAXVGEXAKK

SEQ ID NO: 65 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 9: note = Dbu
GXGKFXKKXKKFGKAXVGEXAKK

SEQ ID NO: 66 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 10: note = Dbu
GXGKFXKKKXKFGKAXVGEXAKK

SEQ ID NO: 67 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 22: note = Dbu
GXGKFXKKKKKFGKAXVGEXAXK

SEQ ID NO: 68 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
GXGXFXKKKKKFGKAXVGEXAKK

SEQ ID NO: 69 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVGEXAKK

SEQ ID NO: 70 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 8: note = Dpr
GXGKFXKXKKKFGKAXVGEXAKK

SEQ ID NO: 71 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 10: note = Dpr
GXGKFXKKKXKFGKAXVGEXAKK

SEQ ID NO: 72 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 11: note = Dpr
GXGKFXKKKKXFGKAXVGEXAKK

SEQ ID NO: 73 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 14: note = Dpr
GXGKFXKKKKKFGXAXVGEXAKK

SEQ ID NO: 74 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 22: note = Dpr
GXGKFXKKKKKFGKAXVGEXAXK

SEQ ID NO: 75 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 23: note = Dpr
GXGKFXKKKKKFGKAXVGEXAKX

SEQ ID NO: 76 - protein, 25 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGE

SEQ ID NO: 77 - protein, 27 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGGE

SEQ ID NO: 78 - protein, 27 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGEE

SEQ ID NO: 79 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGQ

SEQ ID NO: 80 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
  SITE 7..11: note = Dpr
  SITE 14: note = Dpr
GXGXFXXXXXXFGXAXVGEXAKK

SEQ ID NO: 81 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dbu
GXGKFXXKKKKFGKAXVGEXAKK

SEQ ID NO: 82 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVGEXAKKGGE

SEQ ID NO: 83 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 9: note = Dpr
GXGKFXKKXKKFGKAXVGEXAKKGGE

SEQ ID NO: 84 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
  SITE 9: note = Dpr
GXGKFXXKXKKFGKAXVGEXAKK

SEQ ID NO: 85 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
  SITE 9: note = Dpr
GXGKFXXKXKKFGKAXVGEXAKKGGE

SEQ ID NO: 86 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 1: note = L-propargyl glycine
  SITE 4: note = Dpr
  SITE 7..11: note = Dpr
  SITE 14: note = Dpr
  SITE 22..23: note = Dpr
XXGXFXXXXXXFGXAXVGEXAXX

SEQ ID NO: 87 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXSKKKFGKAXVGEXAKK

SEQ ID NO: 88 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFSKAXVGEXAKK

SEQ ID NO: 89 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKSXVGEXAKK

SEQ ID NO: 90 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVSEXAKK

SEQ ID NO: 91 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVGEXSKK

SEQ ID NO: 92 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVGEXAKKGGE

SEQ ID NO: 93 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFSKAXVGEXAKKGGE

SEQ ID NO: 94 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKSXVGEXAKKGGE

SEQ ID NO: 95 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVSEXAKKGGE

SEQ ID NO: 96 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXSKKGGE

SEQ ID NO: 97 - protein, 27 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 27: note = L-propargyl glycine
GXGKFXKKKKKFGKAXVGEXAKKGGEX

SEQ ID NO: 98 - protein, 25 aa, synthetic construct
  VARIANT 1: note = X can be any natural or artificial amino acid
  VARIANT 8: note = X can be any natural or artificial amino acid
  SITE 5: note = Dpr
  SITE 9..12: note = Dpr
  SITE 15: note = Dpr
  SITE 23..24: note = Dpr
XGIGXFLXXXXXFGXAFVGEIAXXG

SEQ ID NO: 99 - protein, 24 aa, synthetic construct
  VARIANT 1: note = X can be any natural or artificial amino acid
  VARIANT 8: note = X can be any natural or artificial amino acid
  SITE 4: note = Dpr
  SITE 7: note = Dpr
  SITE 9..11: note = Dpr
  SITE 14: note = Dpr
  SITE 22..23: note = Dpr
XIGXFLXXXXXFGXAFVGEIAXXG

SEQ ID NO: 100 - protein, 24 aa, synthetic construct
  VARIANT 2: note = X can be any natural or artificial amino acid
  VARIANT 9: note = X can be any natural or artificial amino acid
  SITE 4: note = Dpr
  SITE 7..8: note = Dpr
  SITE 10..11: note = Dpr
  SITE 14: note = Dpr
  SITE 22..23: note = Dpr
GXGXFLXXXXXFGXAFVGEIAXXG

SEQ ID NO: 101 - protein, 24 aa, synthetic construct
  VARIANT 3: note = X can be any natural or artificial amino acid
  VARIANT 10: note = X can be any natural or artificial amino acid
  SITE 4: note = Dpr
  SITE 7..9: note = Dpr
  SITE 11: note = Dpr
  SITE 14: note = Dpr
  SITE 22..23: note = Dpr
GIXXFLXXXXXFGXAFVGEIAXXG

SEQ ID NO: 102 - protein, 24 aa, synthetic construct
  VARIANT 4: note = X can be any natural or artificial amino acid
  SITE 7..10: note = Dpr
  VARIANT 11: note = X can be any natural or artificial amino acid
  SITE 14: note = Dpr
  SITE 22..23: note = Dpr
GIGXFLXXXXXFGXAFVGEIAXXG

SEQ ID NO: 103 - protein, 24 aa, synthetic construct
  VARIANT 5: note = X can be any natural or artificial amino acid
  VARIANT 12: note = X can be any natural or artificial amino acid
  SITE 4: note = Dpr
  SITE 7..11: note = Dpr
  SITE 14: note = Dpr
  SITE 22..23: note = Dpr
GIGXXLXXXXXXGXAFVGEIAXXG

SEQ ID NO: 104 - protein, 24 aa, synthetic construct
  VARIANT 6: note = X can be any natural or artificial amino acid
  VARIANT 13: note = X can be any natural or artificial amino acid
  SITE 4: note = Dpr
  SITE 7..11: note = Dpr
  SITE 14: note = Dpr
  SITE 22..23: note = Dpr
GIGXFXXXXXXFXXAFVGEIAXXG

SEQ ID NO: 105 - protein, 24 aa, synthetic construct
  SITE 4: note = Dpr
  VARIANT 7: note = X can be any natural or artificial amino acid
  SITE 8..11: note = Dpr
  VARIANT 14: note = X can be any natural or artificial amino acid
  SITE 22..23: note = Dpr
GIGXFLXXXXXFGXAFVGEIAXXG

SEQ ID NO: 106 - protein, 24 aa, synthetic construct
  SITE 4: note = Dpr
  SITE 7: note = Dpr
  VARIANT 8: note = X can be any natural or artificial amino acid
  SITE 9..11: note = Dpr
  SITE 14: note = Dpr
  VARIANT 15: note = X can be any natural or artificial amino acid
  SITE 22..23: note = Dpr
GIGXFLXXXXXFGXXFVGEIAXXG

SEQ ID NO: 107 - protein, 24 aa, synthetic construct
  SITE 4: note = Dpr
  SITE 7..8: note = Dpr
  VARIANT 9: note = X can be any natural or artificial amino acid
  SITE 10..11: note = Dpr
  SITE 14: note = Dpr
  VARIANT 16: note = X can be any natural or artificial amino acid
  SITE 22..23: note = Dpr
GIGXFLXXXXXFGXAXVGEIAXXG

SEQ ID NO: 108 - protein, 24 aa, synthetic construct
  SITE 4: note = Dpr
  SITE 7..9: note = Dpr
  VARIANT 10: note = X can be any natural or artificial amino acid
  SITE 11: note = Dpr
  SITE 14: note = Dpr
  VARIANT 17: note = X can be any natural or artificial amino acid
  SITE 22..23: note = Dpr
GIGXFLXXXXXFGXAFXGEIAXXG

SEQ ID NO: 109 - protein, 24 aa, synthetic construct
  SITE 4: note = Dpr
  SITE 7..10: note = Dpr
  VARIANT 11: note = X can be any natural or artificial amino acid
  SITE 14: note = Dpr
  VARIANT 18: note = X can be any natural or artificial amino acid
  SITE 22..23: note = Dpr
GIGXFLXXXXXFGXAFVXEIAXXG

SEQ ID NO: 110 - protein, 24 aa, synthetic construct
  SITE 4: note = Dpr
  SITE 7..11: note = Dpr
  VARIANT 12: note = X can be any natural or artificial amino acid
  SITE 14: note = Dpr
  VARIANT 19: note = X can be any natural or artificial amino acid
  SITE 22..23: note = Dpr
GIGXFLXXXXXXGXAFVGXIAXXG

SEQ ID NO: 111 - protein, 24 aa, synthetic construct
  SITE 4: note = Dpr
  SITE 7..11: note = Dpr
  VARIANT 13: note = X can be any natural or artificial amino acid
  SITE 14: note = Dpr
  VARIANT 20: note = X can be any natural or artificial amino acid
  SITE 22..23: note = Dpr
GIGXFLXXXXXFXXAFVGEXAXXG

SEQ ID NO: 112 - protein, 24 aa, synthetic construct
  SITE 4: note = Dpr
  SITE 7..11: note = Dpr
  VARIANT 14: note = X can be any natural or artificial amino acid
  VARIANT 21: note = X can be any natural or artificial amino acid
  SITE 22..23: note = Dpr
GIGXFLXXXXXFGXAFVGEIXXXG

SEQ ID NO: 113 - protein, 24 aa, synthetic construct
  SITE 4: note = Dpr
  SITE 7..11: note = Dpr
  SITE 14: note = Dpr
  VARIANT 15: note = X can be any natural or artificial amino acid
  VARIANT 22: note = X can be any natural or artificial amino acid
  SITE 23: note = Dpr
GIGXFLXXXXXFGXXFVGEIAXXG

SEQ ID NO: 114 - protein, 24 aa, synthetic construct
  SITE 4: note = Dpr
  SITE 7..11: note = Dpr
  SITE 14: note = Dpr
  VARIANT 16: note = X can be any natural or artificial amino acid
  SITE 22: note = Dpr
  VARIANT 23: note = X can be any natural or artificial amino acid
GIGXFLXXXXXFGXAXVGEIAXXG

SEQ ID NO: 115 - protein, 24 aa, synthetic construct
  SITE 4: note = Dpr
  SITE 7..11: note = Dpr
  SITE 14: note = Dpr
  SITE 22..23: note = Dpr
  VARIANT 24: note = X can be any natural or artificial amino acid
GIGXFLXXXXXFGXAFXGEIAXXX

SEQ ID NO: 116 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
WXGKFXHSKKKFGKAXVGEXAKK

SEQ ID NO: 117 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXWKFXHSKKKFGKAXVGEXAKK

SEQ ID NO: 118 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGWFXHSKKKFGKAXVGEXAKK

SEQ ID NO: 119 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKWXHSKKKFGKAXVGEXAKK

SEQ ID NO: 120 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXWSKKKFGKAXVGEXAKK

SEQ ID NO: 121 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHWKKKFGKAXVGEXAKK

SEQ ID NO: 122 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKWWKAXVGEXAKK

SEQ ID NO: 123 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKWXVGEXAKK

SEQ ID NO: 124 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXWGEXAKK

SEQ ID NO: 125 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVWEXAKK

SEQ ID NO: 126 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGWXAKK

SEQ ID NO: 127 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGEXAKW

SEQ ID NO: 128 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKWGKAXVGEXAKK

SEQ ID NO: 129 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFWKAXVGEXAKK

SEQ ID NO: 130 - protein, 19 aa, synthetic construct
  VARIANT 2: note = X can be any natural or artificial amino acid
  VARIANT 6: note = X can be any natural or artificial amino acid
  VARIANT 12: note = X can be any natural or artificial amino acid
  VARIANT 16: note = X can be any natural or artificial amino acid
GXGKFXKKKKKXVGEXAKK

SEQ ID NO: 131 - protein, 17 aa, synthetic construct
  VARIANT 2: note = X can be any natural or artificial amino acid
  VARIANT 6: note = X can be any natural or artificial amino acid
ZXGKFXHSAKKFGKAFV

SEQ ID NO: 132 - protein, 17 aa, synthetic construct
  VARIANT 2: note = X can be any natural or artificial amino acid
  VARIANT 6: note = X can be any natural or artificial amino acid
ZXGKFXHSKKKFGKAFV

SEQ ID NO: 133 - protein, 17 aa, synthetic construct
  VARIANT 2: note = X can be any natural or artificial amino acid
  VARIANT 6: note = X can be any natural or artificial amino acid
ZXGKFXKSKKKFGKAFV

SEQ ID NO: 134 - protein, 17 aa, synthetic construct
  VARIANT 2: note = X can be any natural or artificial amino acid
  VARIANT 6: note = X can be any natural or artificial amino acid
ZXGKFXKKKKKFGKAFV

SEQ ID NO: 135 - protein, 17 aa, synthetic construct
  VARIANT 2: note = X can be any natural or artificial amino acid
  VARIANT 6: note = X can be any natural or artificial amino acid
  SITE 7: note = Dpr
ZXGKFXXKKKKFGKAFV

SEQ ID NO: 136 - protein, 4 aa, synthetic construct
GGGC

SEQ ID NO: 137 - protein, 4 aa, synthetic construct
GGGE

SEQ ID NO: 138 - protein, 4 aa, synthetic construct
GGEE

SEQ ID NO: 139 - protein, 6 aa, synthetic construct
GGSGGS

SEQ ID NO: 140 - protein, 4 aa, synthetic construct
  VARIANT 1: note = Y is glycine, glutamic acid, or lysine
  VARIANT 2: note = Y is valine, phenylalanine, alanine, tyrosine, or glycine
  VARIANT 3: note = Y is citrulline, arginine, lysine, alanine, or glycine
  VARIANT 4: note = Y is glycine
  VARIANT 1: note = may be absent
  VARIANT 4: note = may be absent
YYYY

SEQ ID NO: 141 - protein, 4 aa, synthetic construct
GGFG

SEQ ID NO: 142 - protein, 12 aa, synthetic construct
  VARIANT 2: note = X can be any natural or artificial amino acid
  SITE 4: note = Dpr
  VARIANT 6: note = X can be any natural or artificial amino acid
  SITE 7..11: note = Dpr
GXGXFXXXXXXF

SEQ ID NO: 143 - protein, 12 aa, synthetic construct
  SITE 2: note = Dpr
  VARIANT 4: note = X can be any natural or artificial amino acid
  VARIANT 8: note = X can be any natural or artificial amino acid
  SITE 10..11: note = Dpr
GXAXVGEXAXXG

SEQ ID NO: 144 - protein, 15 aa, synthetic construct
  VARIANT 2: note = X can be any natural or artificial amino acid
  SITE 4: note = Dpr
  VARIANT 6: note = X can be any natural or artificial amino acid
  SITE 7..11: note = Dpr
  SITE 14: note = Dpr
GXGXFXXXXXXFGXA

SEQ ID NO: 145 - protein, 18 aa, synthetic construct
  SITE 1..5: note = Dpr
  SITE 8: note = Dpr
  VARIANT 10: note = X can be any natural or artificial amino acid
  VARIANT 14: note = X can be any natural or artificial amino acid
  SITE 16..17: note = Dpr
XXXXXFGXAXVGEXAXXG

SEQ ID NO: 146 - skipped

SEQ ID NO: 147 - protein, 20 aa, synthetic construct
  VARIANT 2: note = X can be any natural or artificial amino acid
  SITE 4: note = Dpr
  VARIANT 6: note = X can be any natural or artificial amino acid
  SITE 7..10: note = Dpr
  VARIANT 12: note = X can be any natural or artificial amino acid
  VARIANT 16: note = X can be any natural or artificial amino acid
  SITE 18..19: note = Dpr
GXGXFXXXXXAXVGEXAXXG

SEQ ID NO: 148 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7..11: note = Dpr
GXGAFXXXXXXFGAAXVGEXAAAG

SEQ ID NO: 149 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
  SITE 14: note = Dpr
  SITE 22..23: note = Dpr
GXGXFXAAAAAFGXAXVGEXAXXG

SEQ ID NO: 150 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGAFXAAAAAFGAAXVGEXAAAG

SEQ ID NO: 151 - protein, 24 aa, synthetic construct
  VARIANT 16: note = X can be any natural or artificial amino acid
  VARIANT 20: note = X can be any natural or artificial amino acid
  SITE 2: note = Nle
  SITE 6: note = Nle
  SITE 4: note = Dpr
  SITE 7..11: note = Dpr
  SITE 14: note = Dpr
  SITE 22..23: note = Dpr
GBGXFBXXXXXFGXAXVGEXAXXG

SEQ ID NO: 152 - protein, 24 aa, synthetic construct
  VARIANT 2: note = X can be any natural or artificial amino acid
  VARIANT 6: note = X can be any natural or artificial amino acid
  SITE 4: note = Dpr
  SITE 7..11: note = Dpr
  SITE 14: note = Dpr
  SITE 16: note = Nle
  SITE 20: note = Nle
  SITE 22..23: note = Dpr
GXGXFXXXXXXFGXABVGEBAXXG

SEQ ID NO: 153 - protein, 24 aa, synthetic construct
  SITE 2: note = Nle
  SITE 4: note = Dpr
  SITE 6: note = Nle
  SITE 7..11: note = Dpr
  SITE 14: note = Dpr
  SITE 16: note = Nle
  SITE 20: note = Nle
  SITE 22..23: note = Dpr
GXGXFXXXXXXFGXAXVGEXAXXG

SEQ ID NO: 154 - protein, 24 aa, synthetic construct
  VARIANT 2: note = X can be any natural or artificial amino acid
  VARIANT 6: note = X can be any natural or artificial amino acid
  SITE 7..11: note = Dpr
  VARIANT 16: note = X can be any natural or artificial amino acid
  VARIANT 20: note = X can be any natural or artificial amino acid
GXGSFXXXXXXFGSAXVGEXASSG

SEQ ID NO: 155 - protein, 24 aa, synthetic construct
  VARIANT 2: note = X can be any natural or artificial amino acid
  SITE 4: note = Dpr
  VARIANT 6: note = X can be any natural or artificial amino acid
  SITE 14: note = Dpr
  VARIANT 16: note = X can be any natural or artificial amino acid
  VARIANT 20: note = X can be any natural or artificial amino acid
  SITE 22..23: note = Dpr
GXGXFXSSSSSFGXAXVGEXAXXG

SEQ ID NO: 156 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGSFXSSSSSFGSAXVGEXASSG

SEQ ID NO: 157 - protein, 24 aa, synthetic construct
  VARIANT 2: note = X can be any natural or artificial amino acid
  VARIANT 6: note = X can be any natural or artificial amino acid
  SITE 7..11: note = Dpr
  VARIANT 16: note = X can be any natural or artificial amino acid
  VARIANT 20: note = X can be any natural or artificial amino acid
GXGHFXXXXXXFGHAXVGEXAHHG

SEQ ID NO: 158 - protein, 24 aa, synthetic construct
  VARIANT 2: note = X can be any natural or artificial amino acid
  SITE 4: note = Dpr
  VARIANT 6: note = X can be any natural or artificial amino acid
  SITE 14: note = Dpr
  VARIANT 16: note = X can be any natural or artificial amino acid
  VARIANT 20: note = X can be any natural or artificial amino acid
  SITE 22..23: note = Dpr
GXGXFXHHHHHFGXAXVGEXAXXG

SEQ ID NO: 159 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGHFXHHHHHFGHAXVGEXAHHG

SEQ ID NO: 160-164 - skipped (5 entries)

SEQ ID NO: 165 - protein, 12 aa, synthetic construct
  VARIANT 2: note = X can be any natural or artificial amino acid
  VARIANT 6: note = X can be any natural or artificial amino acid
  SITE 7..11: note = Dpr
GXGSFXXXXXXF

SEQ ID NO: 166 - protein, 12 aa, synthetic construct
  VARIANT 2: note = X can be any natural or artificial amino acid
  VARIANT 6: note = X can be any natural or artificial amino acid
  SITE 4: note = Dpr
GXGXFXHHHHHF

SEQ ID NO: 167 - protein, 12 aa, synthetic construct
  SITE 2: note = Nle
  SITE 4: note = Dpr
  SITE 6: note = Nle
  SITE 7..11: note = Dpr
GXGXFXXXXXXF

SEQ ID NO: 168 - protein, 12 aa, synthetic construct
  SITE 2: note = Nle
  SITE 6: note = Nle
  SITE 7..11: note = Dpr
GXGSFXXXXXXF

SEQ ID NO: 169 - protein, 12 aa, synthetic construct
  SITE 2: note = Nle
  SITE 4: note = Dpr
  SITE 6: note = Nle
GXGXFXHHHHHF

SEQ ID NO: 170 - protein, 20 aa, synthetic construct
  SITE 2: note = Nle
  SITE 4: note = Dpr
  SITE 6: note = Nle
  SITE 7..10: note = Dpr
  SITE 12: note = Nle
  SITE 16: note = Nle
  SITE 18..19: note = Dpr
GXGXFXXXXXAXVGEXAXXG

SEQ ID NO: 171 - protein, 24 aa, synthetic construct
  SITE 2: note = Nle
  SITE 6: note = Nle
  SITE 7..11: note = Dpr
  SITE 16: note = Nle
  SITE 20: note = Nle
GXGSFXXXXXXFGSAXVGEXASSG

SEQ ID NO: 172 - protein, 24 aa, synthetic construct
  SITE 2: note = Nle
  SITE 4: note = Dpr
  SITE 6: note = Nle
  SITE 14: note = Dpr
  SITE 16: note = Nle
  SITE 20: note = Nle
  SITE 22..23: note = Dpr
GXGXFXHHHHHFGXAXVGEXAXXG

SEQ ID NO: 173 - protein, 24 aa, synthetic construct
  VARIANT 2: note = X can be any natural or artificial amino acid
  SITE 4: note = Dpr
  VARIANT 6: note = X can be any natural or artificial amino acid
  SITE 7..11: note = Dpr
  SITE 12: note = D-Phenylalanine
  SITE 14: note = Dpr
  VARIANT 16: note = X can be any natural or artificial amino acid
  VARIANT 20: note = X can be any natural or artificial amino acid
  SITE 22..23: note = Dpr
GXGXFXXXXXXXGXAXVGEXAXXG

SEQ ID NO: 174 - skipped

(19) United States
(12) Patent Application Publication
(10) Pub. No.: US 2026/0191983 A1
(43) Pub. Date: Jul. 9, 2026

(54) COMPOSITIONS AND METHODS FOR PROTEIN INTERNALIZATION

(71) Applicant: Lytica Therapeutics, Inc., Cambridge, MA (US)

(72) Inventors: Rida Mourtada, Cambridge, MA (US); John Ernest Vallarta Bajacan, Cambridge, MA (US); Daniel T. Cohen, Cambridge, MA (US)

(73) Assignee: Lytica Therapeutics, Inc., Cambridge, MA (US)

(21) Appl. No.: 19/133,696

(22) PCT Filed: Dec. 1, 2023
(86) PCT No.: PCT/US2023/082035

§ 371 Date: May 28, 2025

Related U.S. Application Data
(60) Provisional application No. 63/385,867, filed on Dec. 2, 2022.
(60) Provisional application No. 63/579,983, filed on Sep. 1, 2023.

(51) Int. Cl. A61K 47/68; A61P 35/00; C07K 7/06

(52) U.S. Cl. CPC A61K 47/6849; A61K 47/68031; A61K 47/68033; A61K 47/68035; A61K 47/68037; A61K 47/6831; A61P 35/00; C07K 7/06

(57) ABSTRACT

Provided herein are conjugates comprising a promoter peptide conjugated to an agent, such an antibody or antigen-binding fragment thereof. The promoter peptides promote the cellular internalization of the agent (e.g., the antibody or antigen-binding fragment thereof). The promoter peptides, when conjugated to an agent that binds a target protein (e.g., a receptor), can also promote internalization of the target protein into a cell. Also provided are methods of using the conjugates provided herein, e.g., for promoting the uptake of an antibody or antigen-binding fragment thereof into a cell, for promoting the uptake of a target protein into a cell.

### RELATED APPLICATIONS

This application claims priority under 35 U.S.C. § 119(e) to United States provisional patent applications, U.S. Ser. No. 63/385,867, filed Dec. 2, 2022; and U.S. Ser. No. 63/579,983, filed Sep. 1, 2023, the entire contents of each of which are incorporated herein by reference.

### BACKGROUND OF THE INVENTION

The efficacy of some antibody-based therapies, including monoclonal antibodies (mAbs) and antibody-drug conjugates (ADCs), depends not only on binding affinity and specificity of the antibody to a receptor, but also on internalization of the antibody or associated payload into a cell. In the case of ADCs, for example, antibody internalization is often critical for the delivery of cytotoxic agents to intracellular compartments. Despite their binding affinity and specificity for cellular targets, many current antibody-based therapies are limited by low rates of cellular uptake. Methods for promoting cellular uptake of proteins (e.g., antibodies, ADCs) and other agents are therefore important to achieving desired therapeutic effects.

### SUMMARY OF THE INVENTION

The present disclosure is based in part on the discovery that a class of peptides (i.e., “promoter peptides”) can promote the internalization of proteins and other agents into cells, e.g., when the promoter peptide is conjugated to the protein or other agent. Many antibodies, including antigen-binding fragments thereof, for example, are not readily internalized into cells, and therefore, the delivery of such agents to intracellular compartments is often difficult. The present disclosure in one aspect provides conjugates which can promote the internalization of agents (e.g., proteins, such as antibodies) into cells. The conjugates provided herein generally comprise a promoter peptide conjugated to the agent (e.g., protein, such as an antibody). As described herein, the promoter peptide can be a stapled or unstapled peptide.

In one aspect, provided herein are methods of promoting uptake (i.e., internalization) of an agent into a cell, the methods comprising contacting the cell with a conjugate, wherein the conjugate comprises a promoter peptide conjugated to the agent to be delivered; and wherein the promoter peptide is a stapled peptide.

In another aspect, provided herein are methods of promoting uptake (i.e., internalization) of an agent into a cell, the method comprising contacting the cell with a conjugate, wherein the conjugate comprises a promoter peptide conjugated to the agent to be delivered; and wherein the promoter peptide is a Magainin peptide (e.g., a stapled or unstapled Magainin peptide, such as a stapled or unstapled peptide comprising SEQ ID NO: 1 or SEQ ID NO: 2).

In another aspect, provided herein are methods of promoting uptake (i.e., internalization) of an agent into a cell, the method comprising contacting the cell with a conjugate, wherein the conjugate comprises a promoter peptide conjugated to the agent to be delivered; and wherein the promoter peptide is a cationic promoter peptide (e.g., a promoter peptide comprising any one of SEQ ID NOs: 5-19, e.g., any one of SEQ ID NOs: 7-18).

In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the agent to be delivered is a protein such as an antibody or an antigen-binding fragment thereof. In certain embodiments, the agent is not an antibody or antigen-binding fragment thereof. The agent, in certain embodiments, may be any protein that binds a receptor. Other non-limiting examples of agents include proteins such as cytokines and peptide hormones. Additional non-limiting examples of agents include small molecules, polynucleotides, oligonucleotides, and nucleic acids.

In certain embodiments, the agent is an antibody or antigen-binding fragment thereof. In one aspect, provided herein are methods of promoting uptake (i.e., internalization) of an antibody or antigen-binding fragment thereof into a cell, the methods comprising contacting the cell with a peptide-antibody conjugate (PAC), wherein the PAC comprises a promoter peptide conjugated to the antibody or antigen-binding fragment thereof; and wherein the promoter peptide is a stapled peptide.

In another aspect, provided herein are methods of promoting uptake (i.e., internalization) of an antibody or antigen-binding fragment thereof into a cell, the methods comprising contacting the cell with a peptide-antibody conjugate (PAC), wherein the PAC comprises a promoter peptide conjugated to the antibody or antigen-binding fragment thereof; and wherein the promoter peptide is a Magainin peptide (e.g., a stapled or unstapled Magainin peptide, e.g., a stapled or unstapled peptide comprising SEQ ID NO: 1 or SEQ ID NO: 2).

In another aspect, provided herein are methods of promoting uptake (i.e., internalization) of an antibody or antigen-binding fragment thereof into a cell, the methods comprising contacting the cell with a peptide-antibody conjugate (PAC), wherein the PAC comprises a promoter peptide conjugated to the antibody or antigen-binding fragment thereof; and wherein the promoter peptide is a cationic promoter peptide (e.g., a promoter peptide comprising any one of SEQ ID NOs: 5-19, e.g., any one of SEQ ID NOs: 7-18).

In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the percent uptake of the agent (e.g., the antibody or antigen-binding fragment thereof) into the cell is greater than 10%, greater than 25%, greater than 50%, or greater than 75%. In certain embodiments, uptake of the agent (e.g., the antibody or antigen-binding fragment thereof) into the cell is increased by at least 25%, by at least 100%, by at least 400%, or by at least 800%, relative to uptake of the agent not conjugated to the promoter peptide.

In certain embodiments, an antibody or antigen-binding fragment thereof is conjugated to the promoter peptide, and is further conjugated to an additional active agent (i.e., the antibody or antigen-binding fragment thereof is an antibody-drug conjugate (ADC)), and the PAC conjugate promotes internalization of the active agent into a cell. In certain embodiments, the active agent is a small molecule (e.g., a small molecule cytotoxic agent). In certain embodiments, the PAC promotes internalization of the antibody or antigen-binding fragment thereof and the additional active agent (e.g., the small molecule cytotoxic agent).

Accordingly, in another aspect, provided herein are methods of promoting uptake (i.e., internalization) of a small molecule into a cell, the methods comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a stapled peptide.

In another aspect, provided herein are methods of promoting uptake (i.e., internalization) of a small molecule into a cell, the methods comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a Magainin peptide (e.g., a stapled or unstapled Magainin peptide, such as a stapled or unstapled peptide comprising SEQ ID NO: 1 or SEQ ID NO: 2).

In another aspect, provided herein are methods of promoting uptake (i.e., internalization) of a small molecule into a cell, the methods comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a cationic promoter peptide (e.g., a promoter peptide comprising any one of SEQ ID NOs: 5-19, e.g., any one of SEQ ID NOs: 7-18).

In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the small molecule is a small molecule cytotoxic agent. In certain embodiments, the promoter peptide is conjugated to the antibody or antigen-binding fragment thereof of the ADC.

In other embodiments, the small molecule to be delivered (e.g., small molecule cytotoxic agent to be delivered) may be conjugated to the promoter peptide or a linker of a PAC.

In certain embodiments, the percent uptake of the small molecule (e.g., small molecule cytotoxic agent) into the cell is greater than 10%, greater than 25%, greater than 50%, or greater than 75%. In certain embodiments, uptake of the small molecule (e.g., small molecule cytotoxic agent) into the cell is increased by at least 10%, by at least 25%, by at least 50%, by at least 100%, by at least 400%, or by at least 800%, relative to uptake of the small molecule when the ADC is not conjugated to the promoter peptide.

The present disclosure is based on the discovery that the promoter peptides described herein can promote the internalization of extracellular proteins (e.g., receptors) into cells, e.g., when the promoter peptide is conjugated to an agent that binds the extracellular protein (i.e., “target protein”). This discovery led to the development of compositions and methods for the removal of certain target proteins (e.g., receptors) from the outside of the cell (e.g., from the outside of the cell membrane). Significantly, the target protein (e.g., receptor) can be degraded after entry into the cell. In some embodiments, the target protein triggers one or more signaling pathways after entry into the cell.

Therefore, in another aspect, provided herein are methods of promoting uptake (i.e., internalization) of a target protein (e.g., receptor) into a cell, the methods comprising contacting the cell with a conjugate, wherein the conjugate comprises an agent that binds the target protein conjugated to a promoter peptide; and wherein the promoter peptide is a stapled peptide.

In another aspect, provided herein are methods of promoting uptake (i.e., internalization) of a target protein into a cell, the methods comprising contacting the cell with a conjugate, wherein the conjugate comprises an agent that binds the target protein conjugated to a promoter peptide; and wherein the promoter peptide is a Magainin peptide (e.g., a stapled or unstapled Magainin peptide, e.g., a stapled or unstapled peptide comprising SEQ ID NO: 1 or SEQ ID NO: 2).

In another aspect, provided herein are methods of promoting uptake (i.e., internalization) of a target protein into a cell, the methods comprising contacting the cell with a conjugate, wherein the conjugate comprises an agent that binds the target protein conjugated to a promoter peptide; and wherein the promoter peptide is a cationic promoter peptide (e.g., a promoter peptide comprising any one of SEQ ID NOs: 5-19, e.g., any one of SEQ ID NOs: 7-18).

In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the target protein is on the surface of the cell. In certain embodiments, the target protein is a receptor or a fragment thereof (e.g., HER2, EGFR, or a fragment thereof). In certain embodiments, the agent that binds the target protein is an antibody or antigen-binding fragment thereof, i.e., the cell is contacted with a PAC described herein. In some embodiments, the agent that binds the target protein is a ligand of the receptor, such as a peptide ligand.

In certain embodiments, the percent uptake of the target protein (e.g., receptor) into the cell is greater than 10%, greater than 25%, greater than 50%, or greater than 75%. In certain embodiments, uptake of the target protein (e.g., receptor) into the cell is increased by at least 25%, by at least 100%, by at least 400%, or by at least 800%, relative to uptake of the target protein when the agent that binds the target protein is not conjugated to the promoter peptide. In some embodiments, the target protein (e.g., receptor) is degraded after entering the cell. In some embodiments, the target protein triggers one or more signaling pathways after entry into the cell.

In certain embodiments of any of the methods provided herein, the cell is in a subject, and the method comprises administering to the subject an effective amount (e.g., a therapeutically effective amount) of a conjugate described herein (e.g., a PAC described herein). In certain embodiments, the subject is diagnosed with a disease, such as cancer or a tumor.

Conjugates (e.g., PACs) are also provided herein, as well as pharmaceutical compositions and kits comprising the same. Methods of preparing conjugates (e.g., PACs) described herein are also provided.

Non-limiting examples of peptide-antibody conjugates (PACs) are provided in in Table 2A and Table 2B. Other non-limiting examples of PACs are provided in Table 2C, Table 3C, and Table 3D (infra).

TABLE 2A
Examples of PACs with Stapled Promoter Peptides

| | Promoter | | | Promoter | | |
|---|---|---|---|---|---|---|
| | Peptide | X¹-X² | X³-X⁴ | Peptide | | |
| | SEQ ID | cross- | Cross- | C- | Linker | |
| PAC# | NO: | link | link | Terminus | Number | Antibody |
| 1 | 2 | alk | alk | —NH₂ | L5 | daratumumab |
| 2 | 2 | alk | alk | —NH₂ | L5 | obinutuzumab |
| 3 | 2 | alk | alk | —NH₂ | L5 | trastuzumab |
| 4 | 2 | alk | alk | —NH₂ | L5 | cetuximab |

TABLE 2B
Examples of PACs with Unstapled Promoter Peptides

| | | | | Promoter | Promoter |
|---|---|---|---|---|---|
| | | Linker | | Peptide | Peptide C- |
| PAC# | Antibody | Number | Promoter Peptide Sequence | SEQ ID NO: | Terminus |
| 5 | daratumumab | L11 | (DapFADapFADap)₂DapFADap | 15 | —NH₂ |
| 6 | daratumumab | L11 | (DapFADapFADap)₂ | 8 | —NH₂ |
| 7 | daratumumab | L9 | (DapFADapFADap)₂DapFADap | 15 | —NH₂ |
| 8 | daratumumab | L11 | (DapYADapYADap)₂DapYADap | 16 | —NH₂ |
| 9 | daratumumab | L11 | (DapFGDapFGDap)₂DapFGDap | 17 | —NH₂ |
| 10 | daratumumab | L11 | (DapFSDapFSDap)₂DapFSDap | 18 | —NH₂ |

The details of certain embodiments of the invention are set forth in the Detailed Description of Certain Embodiments, as described below. Other features, objects, and advantages of the invention will be apparent from the Definitions, Examples, Figures, and Claims.

### Definitions

### General Definitions

The following definitions are general terms used throughout the present application.

The terms “peptide” and “polypeptide” are used interchangeably and refer to a polymer of amino acid residues linked together by peptide bonds. The terms also include “proteins,” and refer to peptides, polypeptides, and proteins, of any size, structure, or function. Typically, a peptide will be at least three amino acids long, or at least the length required by an amino acid sequence provided herein. A peptide may refer to an individual peptide or a collection of peptides. Peptides provided herein can include natural amino acids and/or unnatural amino acids (i.e., compounds that do not occur in nature but that can be incorporated into a peptide chain) in any combination. One or more of the amino acids in a peptide may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A peptide may be a fragment or modified version of a naturally occurring peptide or protein. A peptide may be naturally occurring, recombinant, synthetic, or any combination of these.

“Promoter peptide,” as used herein, refers to a peptide capable of promoting the internalization of an agent (e.g., protein) to which it is conjugated into a cell. In certain embodiments, for example, a promoter peptide is capable of promoting the internalization of an antibody or antigen-binding fragment thereof to which it is conjugated into a cell. A promoter peptide can be a stapled or unstapled peptide. In certain embodiments, the promoter peptide is a stapled or unstapled Magainin peptide.

Throughout the present disclosure, references to a “promoter peptide” is intended to encompass, without limitation, peptides comprising any amino acid sequence provided herein and pharmaceutically acceptable salts, stereoisomers, tautomers, isotopically labeled derivatives, solvates, hydrates, polymorphs, co-crystals, and prodrugs thereof.

“Internalization” and “uptake” are used interchangeably and refer to the movement of an entity (e.g., agent, such as an antibody, or target protein) from an extracellular location (i.e., a location outside of the cell membrane) to an intracellular location (i.e., a location within the cell). Internalization and uptake, as used herein, are not limited to a particular mechanism.

“Percent uptake,” when referring to uptake of an agent, refers to the percent of the agent (e.g., an antibody) that is internalized into a cell relative to the total amount of the agent that is exposed to the cell. “Percent uptake,” when referring to uptake of a target protein (e.g., receptor), refers to the percent of the target protein (e.g., receptor) that is internalized into a cell relative to the total amount of the target protein on the surface of or external to the cell.

The term “amino acid” refers to a molecule containing both an amino group and a carboxyl group. Amino acids include alpha-amino acids, the generic structure of which is depicted below. Each amino acid referred to herein may be denoted by a 1- to 4-letter code (e.g., R and Arg represent L-Arginine, hArg represents L-homoarginine).

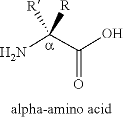

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided below), and unnatural alpha-amino acids.

Exemplary natural alpha-amino acids (with one-letter code provided in parentheses) include L-alanine (A), L-arginine (R), L-asparagine (N), L-aspartic acid (D), L-cysteine (C), L-glutamic acid (E), L-glutamine (Q), glycine (G), L-histidine (H), L-isoleucine (I), L-leucine (L), L-lysine (K), L-methionine (M), L-phenylalanine (F), L-proline (P), L-serine (S), L-threonine (T), L-tryptophan (W), L-tyrosine (Y), and L-valine (V).

Exemplary unnatural alpha-amino acids include D-arginine, D-asparagine, D-aspartic acid, D-cysteine, D-glutamic acid, D-glutamine, D-histidine, D-isoleucine, D-leucine, D-lysine, D-methionine, D-phenylalanine, D-proline, D-serine, D-threonine, D-tryptophan, D-tyrosine, D-valine, Di-vinyl, α-methyl-alanine (Aib), α-methyl-arginine, α-methyl-asparagine, α-methyl-aspartic acid, α-methyl-cysteine, α-methyl-glutamic acid, α-methyl-glutamine, α-methyl-histidine, α-methyl-isoleucine, α-methyl-leucine, α-methyl-lysine, α-methyl-methionine, α-methyl-phenylalanine, α-methyl-proline, α-methyl-serine, α-methyl-threonine, α-methyl-tryptophan, α-methyl-tyrosine, α-methyl-valine, norleucine, and terminally unsaturated alpha-amino acids. There are many known unnatural amino acids any of which may be included in the peptides of the present disclosure. See for example, S. Hunt, The Non-Protein Amino Acids: In Chemistry and Biochemistry of the Amino Acids, edited by G. C. Barrett, Chapman and Hall, 1985. Unnatural amino acids also include amino acids comprising nitrogen substituents.

Certain amino acids referred to herein are provided in Table 1 below (represented by name, structure, and 1- to 4-letter code).

TABLE 1
Certain Amino Acids

| Name | Code | Structure |
|---|---|---|
| L-Norleucine | B | 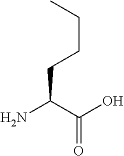 |
| L-Ornithine | Orn | 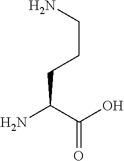 |
| L-Diaminobutyric Acid | Dab | 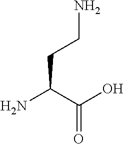 |
| L-Diaminopropionic Acid | Dap | 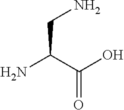 |
| ß-Alanine | BAla | 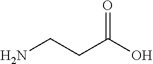 |
| L-Homoarginine | hArg | 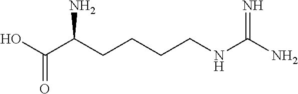 [SMILES: N=C(N)NCCCC[C@H](N)C(=O)O] |
| D-Phenylalanine | F¹ | 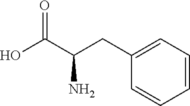 [SMILES: N[C@H](Cc1ccccc1)C(=O)O] |
| L-4-trifluoromethyl phenylalanine | F² | 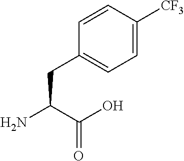 [SMILES: N[C@@H](Cc1ccc(C(F)(F)F)cc1)C(=O)O] |
| L-4-t-butyl phenylalanine | F³ | 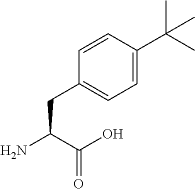 [SMILES: CC(C)(C)c1ccc(C[C@H](N)C(=O)O)cc1] |
| L-4-fluoro phenylalanine | F⁴ | 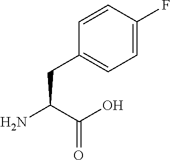 [SMILES: N[C@@H](Cc1ccc(F)cc1)C(=O)O] |
| L-4-methyl phenylalanine | F⁵ | 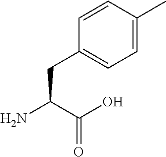 |
| S-2-(4′-pentenyl) alanine | S⁵ | 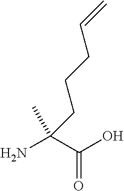 |
| R-2-(7′-octenyl) alanine | R⁸ | 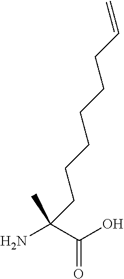 |
| L-propargyl glycine | J | 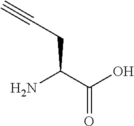 |
| L-Azidolysine | Azi | 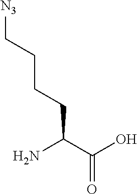 [SMILES: [N-]=[N+]=NCCCC[C@H](N)C(=O)O] |

“Modified phenylalanine” for the purpose of this disclosure means a stereoisomer of phenylalanine (e.g., D-Phe) or any unnatural phenylalanine analog wherein the phenyl ring is ortho-, meta-, and/or para-substituted with one or more non-hydrogen substituents (e.g., optionally substituted alkyl (e.g., Me, Et, n-Pr, i-Pr, n-Bu, t-Bu), halogen (e.g., F, Cl, Br, I), haloalkyl (e.g., —CF₃), optionally substituted hydroxyl (e.g., —OH), optionally substituted amino (e.g., —NH₂), optionally substituted thio (e.g., —SH), optionally substituted acyl (e.g., —C(═O)Me, —C(═O)OH, —C(═O)NH₂), —CN, —SCN, —NO₃, —N₃). Examples of modified phenylalanines include, but are not limited to, F¹, F⁵, F⁴, F², and F³.

The term “amino acid substitution” when used in reference to an amino acid sequence refers to an amino acid of the amino acid sequence being replaced by a different amino acid (e.g., replaced by a natural or unnatural amino acid). An amino acid sequence provided herein may comprise or include one or more amino acid substitutions. Specific amino acid substitutions are denoted by commonly used colloquial nomenclature in the art of peptide sequencing to denote amino acid sequence variations. For example, when referring to SEQ ID NO: 20 (below), an “amino acid substitution at H7” refers to the histidine (H) at position 7 of the amino acid sequence being replaced by a different amino acid (e.g., a natural or unnatural amino acid other than histidine). Also, for example, when referring to SEQ ID NO: 20, the amino acid substitution “H7K” refers to replacing the histidine (H) at position 7 of the amino acid sequence of SEQ ID NO: 20 with lysine (K), resulting in an amino acid sequence represented by SEQ ID NO: 21 (below).

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | (Position #) |
|---|---|---|---|---|---|---|---|---|---|---|---|---|---|---|---|---|---|---|---|---|---|---|---|
| G | X¹ | G | K | F | X² | H | K | K | K | K | F | G | K | A | X³ | V | G | E | X⁴ | A | K | K | SEQ ID NO: 20 |
| G | X¹ | G | K | F | X² | K | K | K | K | K | F | G | K | A | X³ | V | G | E | X⁴ | A | K | K | SEQ ID NO: 21 |

The term “amino acid addition” when used in reference to an amino acid sequence refers to an amino acid (e.g., a natural or unnatural amino acid) being inserted between two amino acids of the amino acid sequence, or added at either end of the sequence. Standard colloquial nomenclature is used to represent specific amino additions (e.g., when referring to SEQ ID NO: 20, “G3_K4insX” denotes that a hypothetical amino acid X is inserted between amino acids G3 and K4 of the amino acid sequence). In certain embodiments, an amino acid sequence herein can comprise 0, 1, 2, 3, 4, 5, 6, 7, 8, 9, or 10 amino acid additions.

The term “amino acid deletion” when used in reference to an amino acid sequence refers to an amino acid of the amino acid sequence being deleted from the amino acid sequence. Standard colloquial nomenclature is used to represent specific amino deletions (e.g., when referring to SEQ ID NO: 20, “G1del” denotes that the amino acid G1 is deleted from the sequence). In certain embodiments, an amino acid sequence herein can comprise 0, 1, 2, 3, 4, 5, 6, 7, 8, 9, or 10 amino acid deletions.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the peptides of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the peptides of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a conjugate described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a conjugate (e.g., PAC) described herein refers to an amount sufficient to elicit the desired biological response. An effective amount of a conjugate (e.g., PAC) described herein may vary depending on such factors as the desired biological endpoint, severity of side effects, disease, or disorder, the identity, pharmacokinetics, and pharmacodynamics of the particular peptide, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a conjugate (e.g., PAC) described herein in a single dose. In certain embodiments, an effective amount is the combined amounts of a conjugate (e.g., PAC) described herein in multiple doses.

A “therapeutically effective amount” of a conjugate (e.g., PAC) described herein is an amount sufficient to provide a therapeutic benefit in the treatment of a condition or to delay or minimize one or more symptoms associated with the condition. A therapeutically effective amount of a conjugate (e.g., PAC) means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a conjugate (e.g., PAC) described herein is an amount sufficient to prevent a condition, or one or more symptoms associated with the condition or prevent its recurrence. A prophylactically effective amount of a conjugate (e.g., PAC) means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Peptides described herein can comprise one or more asymmetric centers, and thus can exist in various stereoisomeric forms, e.g., enantiomers and/or diastereomers. For example, the peptides described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses peptides as individual isomers substantially free of other isomers, and alternatively, as mixtures of various isomers.

In a formula, the bond [symbol] is a single bond, the dashed line - - - is a single bond or absent, and the bond [symbol] or [symbol] is a single or double bond. Additionally, the bond [symbol] or [symbol] is a double or triple bond.

Unless otherwise provided, formulae and structures depicted herein include peptides that do not include isotopically enriched atoms, and also include peptides that include isotopically enriched atoms (“isotopically labeled derivatives”). For example, peptides having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such peptides are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₃, C₂₋₅, C₂₋₄, C₂₋₃, C₃₋₆, C₃₋₅, C₃₋₄, C₄₋₃, C₄₋₅, and C₅₋₆ alkyl.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like.

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). The one or more carbon-carbon double bonds can be internal (such as in 2-butenyl) or terminal (such as in 1-butenyl). In an alkenyl group, a C═C double bond for which the stereochemistry is not specified (e.g., —CH═CHCH₃ or

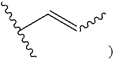

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”).

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). The one or more carbon-carbon triple bonds can be internal (such as in 2-butynyl) or terminal (such as in 1-butynyl).

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”).

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 6 ring carbon atoms (“C₃₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused to one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom or the ring that does not contain a heteroatom.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A chemical moiety is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, heteroaryl, acyl groups are optionally substituted. In general, the term “substituted” when referring to a chemical group means that at least one hydrogen present on the group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The invention is not limited in any manner by the exemplary substituents described herein.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SCN, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, S(═O)R^{aa}, —OS(═O)R^{aa}, —Si(R^{aa})₃, —OSi(R^{aa})₃—C(═S)N(R^{bb})₂, —C(═O)SR^{aa}, C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;
   each instance of RC is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring; and each X⁻ is a counterion.

In certain embodiments, each substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —N₃, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S—SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen. The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen. The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent.

Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻).Exemplary counterions which may be multivalent include CO₃²⁻, HPO₄²⁻, PO₄³⁻, B₄O₇²⁺, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, Figures, and Claims. The invention is not limited in any manner by the above exemplary listing of substituents.

### BRIEF DESCRIPTION OF THE DRAWINGS

The accompanying drawings, which are incorporated in and constitute a part of this specification, illustrate several embodiments of the invention and together with the description, provide non-limiting examples of the invention.

FIG. 1A. Anti-proliferative activity of PAC1 in various cell lines. FIG. 1B. Anti-proliferative activity of daratumumab (not conjugated to a promoter peptide) in various cell lines.

FIG. 2A. CD38 RNA expression in various cell lines derived from DepMap database. FIG. 2B. Anti-CD38 antibody (daratumumab; “Dara”) uptake in CD38+ cell line SUDHL8; unconjugated daratumumab compared to PAC1.

FIG. 3. Anti-CD20 antibody (obinutuzumab; “Obi”) uptake in a CD20+ cell line SUDHL4; unconjugated obinutuzumab compared to PAC2.

FIG. 4. Anti-HER2 antibody (trastuzumab; “Trast”) uptake in a HER2+ cell line SKOV3; unconjugated trastuzumab compared to PAC3.

FIG. 5. Time course of degradation of EGFR in an EGFR+ cell line (COL0678) with PAC4. Lanes from left to right: Time 0, 1 hour post-treatment, 4 hours post-treatment, 8 hours post-treatment, 24 hours post-treatment.

FIG. 6. Anti-CD38 antibody (daratumumab; “Dara”) uptake in CD38+ cell line SUDHL4; unconjugated daratumumab compared to PAC1, PAC5, and PAC6.

FIG. 7. Antibody internalization for PAC1 with different peptide-antibody ratios over time.

FIG. 8. Antibody internalization with different linkers over time. PAR=Peptide-antibody ratio.

FIG. 9. EGFR degradation with various stapled promoter peptides conjugated to Cetuximab at 24 hours post-treatment.

FIG. 10. EGFR degradation with full antibody and Fab fragment conjugated to a promoter peptide at 24 hours post-treatment with cycloheximide co-treatment. PAR=peptide-antibody ratio.

### DETAILED DESCRIPTION OF CERTAIN EMBODIMENTS

Provided herein are conjugates comprising a promoter peptide conjugated to an agent, such an antibody or antigen-binding fragment thereof. The promoter peptides promote the internalization of the agent (e.g., the antibody or antigen-binding fragment thereof) into a cell. The promoter peptides, when conjugated to an agent that binds a target protein (e.g., a receptor), can also promote internalization of the target protein itself. Also provided are methods of using the conjugates provided herein, e.g., for promoting the uptake of an antibody or antigen-binding fragment thereof into a cell, for promoting the uptake of a target protein (e.g., a receptor) into a cell.

### Promoting Internalization of Proteins

In one aspect, provided herein are methods of promoting uptake of an agent (e.g., a protein) into a cell, the method comprising contacting the cell with a conjugate, wherein the conjugate comprises a promoter peptide conjugated to the agent to be delivered. In certain embodiments, the promoter peptide is a stapled peptide. In certain embodiments, the promoter peptide is a Magainin peptide (e.g., a stapled or unstapled Magainin peptide). In certain embodiments, the promoter peptide is a cationic promoter peptide (e.g., a promoter peptide comprising any one of SEQ ID NOs: 5-19, e.g., any one of SEQ ID NOs: 7-18). In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the agent to be delivered is a protein. In certain embodiments, the agent is an antibody or antigen-binding fragment thereof. In certain embodiments, the agent is not an antibody or antigen-binding fragment thereof. In certain embodiments, the agent is a protein that binds a receptor (e.g., HER2, EGFR, GPCR, PCKS9, Nav1.7). In certain embodiments, the agent is a cytokine. In certain embodiments, the agent is a peptide hormone. Other non-limiting examples of agents include small molecules, polynucleotides, oligonucleotides, and nucleic acids.

As described herein, in certain embodiments, the agent is a protein, such an antibody or antigen-binding fragment thereof. In one aspect, provided herein are methods of promoting uptake (i.e., internalization) of an antibody or antigen-binding fragment thereof into a cell, the methods comprising contacting the cell with a peptide-antibody conjugate (PAC), wherein the PAC comprises a promoter peptide conjugated to the antibody or antigen-binding fragment thereof.

In certain embodiments, provided herein are methods of promoting uptake (i.e., internalization) of an antibody or antigen-binding fragment thereof into a cell, the methods comprising contacting the cell with a peptide-antibody conjugate (PAC), wherein the PAC comprises a promoter peptide conjugated to the antibody or antigen-binding fragment thereof; and wherein the promoter peptide is a stapled peptide.

In certain embodiments, provided herein are methods of promoting uptake (i.e., internalization) of an antibody or antigen-binding fragment thereof into a cell, the methods comprising contacting the cell with a peptide-antibody conjugate (PAC), wherein the PAC comprises a promoter peptide conjugated to the antibody or antigen-binding fragment thereof, and wherein the promoter peptide is a Magainin peptide (e.g., a stapled or unstapled Magainin peptide, e.g., a stapled or unstapled peptide comprising SEQ ID NO: 1 or SEQ ID NO: 2).

In certain embodiments, provided herein are methods of promoting uptake (i.e., internalization) of an antibody or antigen-binding fragment thereof into a cell, the methods comprising contacting the cell with a peptide-antibody conjugate (PAC), wherein the PAC comprises a promoter peptide conjugated to the antibody or antigen-binding fragment thereof; and wherein the promoter peptide is a cationic promoter peptide (e.g., a promoter peptide comprising any one of SEQ ID NOs: 5-19, e.g., any one of SEQ ID NOs: 7-18).

In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the cell is contacted in vivo (i.e., in a subject). In certain embodiments, the cell is contacted in vitro (e.g., in a cellular assay, in culture).

In certain embodiments, the percent uptake of the agent to be delivered (e.g., the antibody or antigen-binding fragment thereof) into the cell is greater than 10% (e.g., greater than 20%, greater than 25%, greater than 30%, greater than 35%, greater than 40%, greater than 45%, greater than 50%, greater than 60%, greater than 65%, or greater than 70%, or greater than 75%, or greater than 80%, or greater than 85%, greater than 90%, or greater than 95%). In certain embodiments, the percent uptake of the agent (e.g., the antibody or antigen-binding fragment thereof) into the cell is greater than 25%. In certain embodiments, the percent uptake of the agent (e.g., the antibody or antigen-binding fragment thereof) into the cell is greater than 50%. In certain embodiments, the percent uptake of the agent (e.g., the antibody or antigen-binding fragment thereof) into the cell is greater than 75%.

In certain embodiments, uptake of the agent (e.g., the antibody or antigen-binding fragment thereof) into the cell is increased by at least 10% (e.g., by at least 25%, by at least 50%, by at least 75%, by at least 100%, by at least 200%, by at least 300%, by at least 400%, by at least 500%, by at least 600%, by at least 700%, by at least 800%, by at least 900%, by at least 1000%), relative to uptake of the agent (e.g., the antibody or antigen-binding fragment thereof) not conjugated to the promoter peptide. In certain embodiments, uptake of the agent (e.g., the antibody or antigen-binding fragment thereof) into the cell is increased by at least 25%, relative to uptake of the agent (e.g., the antibody or antigen-binding fragment thereof) not conjugated to the promoter peptide. In certain embodiments, uptake of the agent (e.g., the antibody or antigen-binding fragment thereof) into the cell is increased by at least 100%, relative to uptake of the agent (e.g., the antibody or antigen-binding fragment thereof) not conjugated to the promoter peptide. In certain embodiments, uptake of the agent (e.g., the antibody or antigen-binding fragment thereof) into the cell is increased by at least 200%, relative to uptake of the agent (e.g., the antibody or antigen-binding fragment thereof) not conjugated to the promoter peptide. In certain embodiments, uptake of the agent (e.g., the antibody or antigen-binding fragment thereof) into the cell is increased by at least 400%, relative to uptake of the agent (e.g., the antibody or antigen-binding fragment thereof) not conjugated to the promoter peptide. In certain embodiments, uptake of the agent (e.g., the antibody or antigen-binding fragment thereof) into the cell is increased by at least 600%, relative to uptake of the agent (e.g., the antibody or antigen-binding fragment thereof) not conjugated to the promoter peptide. In certain embodiments, uptake of the agent (e.g., the antibody or antigen-binding fragment thereof) into the cell is increased by at least 800%, relative to uptake of the agent (e.g., the antibody or antigen-binding fragment thereof) not conjugated to the promoter peptide. In certain embodiments, uptake of the agent (e.g., the antibody or antigen-binding fragment thereof) into the cell is increased by at least 1000%, relative to uptake of the agent (e.g., the antibody or antigen-binding fragment thereof) not conjugated to the promoter peptide.

In certain embodiments, the promoter peptide promotes uptake of the agent (e.g., the antibody or antigen-binding fragment thereof) into the cell, whereas the agent (e.g., the antibody or antigen-binding fragment thereof) not conjugated to the promoter peptide is not internalized into a cell in a statistically significant amount.

In certain embodiments, the antibody is an antibody directed against CD38, or an antigen-binding fragment thereof. In certain embodiments, the antibody is a monoclonal antibody (mAb) directed against CD38, or an antigen-binding fragment thereof. In certain embodiments, the antibody is daratumumab, or an antigen-binding fragment thereof. In certain embodiments, the antibody is daratumumab. Other non-limiting examples of anti-CD38 antibodies are provided herein.

In certain embodiments, the antibody is an antibody directed against CD20, or an antigen-binding fragment thereof. In certain embodiments, the antibody is a monoclonal antibody (mAb) directed against CD20, or an antigen-binding fragment thereof. In certain embodiments, the antibody is obinutuzumab, or an antigen-binding fragment thereof. In certain embodiments, the antibody is obinutuzumab. In certain embodiments, the antibody is rituximab, or an antigen-binding fragment thereof. In certain embodiments, the antibody is rituximab. Other non-limiting examples of anti-CD20 antibodies are provided herein.

In certain embodiments, the antibody is an antibody directed against HER2, or an antigen-binding fragment thereof. In certain embodiments, the antibody is a monoclonal antibody (mAb) directed against HER2, or an antigen-binding fragment thereof. In certain embodiments, the antibody is trastuzumab, or an antigen-binding fragment thereof. In certain embodiments, the antibody is trastuzumab. Other non-limiting examples of anti-HER2 antibodies are provided herein.

In certain embodiments, the antibody is an antibody directed against HER3, or an antigen-binding fragment thereof. In certain embodiments, the antibody is a monoclonal antibody (mAb) directed against HER3, or an antigen-binding fragment thereof. In certain embodiments, the antibody is patritumab, or an antigen-binding fragment thereof. In certain embodiments, the antibody is patritumab. Other non-limiting examples of anti-HER3 antibodies are provided herein.

In certain embodiments, the antibody is an antibody directed against EGFR, or an antigen-binding fragment thereof. In certain embodiments, the antibody is a monoclonal antibody (mAb) directed against EGFR, or an antigen-binding fragment thereof. In certain embodiments, the antibody is cetuximab, or an antigen-binding fragment thereof. In certain embodiments, the antibody is cetuximab. Other non-limiting examples of anti-EGFR antibodies are provided herein.

In certain embodiments, the antibody is an antibody directed against Folate Receptor Alpha, or an antigen-binding fragment thereof. In certain embodiments, the antibody is a monoclonal antibody (mAb) directed against Folate Receptor Alpha, or an antigen-binding fragment thereof. In certain embodiments, the antibody is mirvetuximab, or an antigen-binding fragment thereof. In certain embodiments, the antibody is mirvetuximab.

Promoter peptides are described herein. In certain embodiments, the promoter peptide is a stapled peptide (e.g., a singly stapled, doubly stapled, or stitched peptide). In certain embodiments, the promoter peptide is a stapled Magainin peptide. In certain embodiments, the promoter peptide is a stapled Magainin II peptide.

In certain embodiments, the promoter peptide is a stapled peptide comprising SEQ ID NO: 1 (wherein X¹ and X² are connected via a crosslink and X³ and X⁴ are connected via a crosslink). In certain embodiments, the promoter peptide is a stapled peptide comprising SEQ ID NO: 2 (wherein X¹ and X² are connected via a crosslink and X³ and X⁴ are connected via a crosslink). In certain embodiments, the promoter peptide comprises any one of SEQ ID NOs: 1-21, 27-135, and 142-174 (wherein X¹ and X² are connected via a crosslink, and X³ and X⁴ are connected via a crosslink, where applicable). In certain embodiments, the promoter peptide comprises any one of SEQ ID NOs: 142-174 (wherein X¹ and X² are connected via a crosslink, and X³ and X⁴ are connected via a crosslink, where applicable).

In certain embodiments, the antibody is a monoclonal antibody (mAb) directed against CD38, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the antibody is daratumumab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the antibody is a monoclonal antibody (mAb) directed against CD38, or an antigen-binding fragment thereof; and the promoter peptide comprises any one of SEQ ID NOs: 142-174. In certain embodiments, the antibody is daratumumab, or an antigen-binding fragment thereof; and the promoter peptide comprises any one of SEQ ID NOs: 142-174.

In certain embodiments, the antibody is a monoclonal antibody (mAb) directed against CD20, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the antibody is obinutuzumab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the antibody is a monoclonal antibody (mAb) directed against HER2, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the antibody is trastuzumab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the antibody is a monoclonal antibody (mAb) directed against HER3, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the antibody is patritumab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the antibody is a monoclonal antibody (mAb) directed against HER3, or an antigen-binding fragment thereof; and the promoter peptide comprises any one of SEQ ID NOs: 142-174. In certain embodiments, the antibody is patritumab, or an antigen-binding fragment thereof; and the promoter peptide comprises any one of SEQ ID NOs: 142-174.

In certain embodiments, the antibody is a monoclonal antibody (mAb) directed against EGFR, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the antibody is cetuximab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the antibody is a monoclonal antibody (mAb) directed against Folate Receptor Alpha, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the antibody is mirvetuximab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the antibody is a monoclonal antibody (mAb) directed against Folate Receptor Alpha, or an antigen-binding fragment thereof; and the promoter peptide comprises any one of SEQ ID NOs: 142-174. In certain embodiments, the antibody is mirvetuximab, or an antigen-binding fragment thereof; and the promoter peptide comprises any one of SEQ ID NOs: 142-174.

In certain embodiments, the antibody is a monoclonal antibody (mAb) directed against CD38, or an antigen-binding fragment thereof; and the promoter peptide comprises any one of SEQ ID NOs: 5-19. In certain embodiments, the antibody is a monoclonal antibody (mAb) directed against CD38, or an antigen-binding fragment thereof; and the promoter peptide comprises any one of SEQ ID NOs: 7-18.

Additional embodiments and non-limiting examples of peptide-antibody conjugates (PACs) are provided herein, including in Table 2A, Table 2B, and Table 2C.

TABLE 2A
Examples of PACs with Stapled Promoter Peptides

| | Promoter | | | Promoter | | |
|---|---|---|---|---|---|---|
| | Peptide | X¹-X² | X³-X⁴ | Peptide | | |
| | SEQ ID | cross- | Cross- | C- | Linker | |
| PAC# | NO: | link | link | Terminus | Number | Antibody |
| 1 | 2 | alk | alk | —NH₂ | L5 | daratumumab |
| 2 | 2 | alk | alk | —NH₂ | L5 | obinutuzumab |
| 3 | 2 | alk | alk | —NH₂ | L5 | trastuzumab |
| 4 | 2 | alk | alk | —NH₂ | L5 | cetuximab |

TABLE 2B
Examples of PACs with Unstapled Promoter Peptides

| | | | | Promoter | Promoter |
|---|---|---|---|---|---|
| | | Linker | | Peptide | Peptide C- |
| PAC# | Antibody | Number | Promoter Peptide Sequence | SEQ ID NO: | Terminus |
| 5 | daratumumab | L11 | (DapFADapFADap)₂DapFADap | 15 | —NH₂ |
| 6 | daratumumab | L11 | (DapFADapFADap)₂ | 8 | —NH₂ |
| 7 | daratumumab | L9 | (DapFADapFADap)₂DapFADap | 15 | —NH₂ |
| 8 | daratumumab | L11 | (DapYADapYADap)₂DapYADap | 16 | —NH₂ |
| 9 | daratumumab | L11 | (DapFGDapFGDap)₂DapFGDap | 17 | —NH₂ |
| 10 | daratumumab | L11 | (DapFSDapFSDap)₂DapFSDap | 18 | —NH₂ |

TABLE 2C
Examples of PACs with Stapled or Unstapled Promoter Peptides

| | | | Promoter | | | Promoter | |
|---|---|---|---|---|---|---|---|
| | | | Peptide SEQ | X¹-X² | X³-X⁴ | Peptide C- | |
| PAC# | Antibody | Linker | ID NO: | Crosslink | Crosslink | Terminus | Target |
| 11 | Daratumumab | L5 | 142 | alk | N/A | —NH₂ | CD38 |
| 12 | Daratumumab | L5 | 143 | N/A | alk | —NH₂ | CD38 |
| 13 | Daratumumab | L5 | 144 | alk | N/A | —NH₂ | CD38 |
| 14 | Daratumumab | L5 | 145 | N/A | alk | —NH₂ | CD38 |
| 15 | Daratumumab | L5 | 146 | N/A | N/A | —NH₂ | CD38 |
| 16 | Daratumumab | L5 | 147 | alk | alk | —NH₂ | CD38 |
| 17 | Daratumumab | L5 | 148 | alk | alk | —NH₂ | CD38 |
| 18 | Daratumumab | L5 | 149 | alk | alk | —NH₂ | CD38 |
| 19 | Daratumumab | L5 | 151 | N/A | alk | —NH₂ | CD38 |
| 20 | Daratumumab | L5 | 152 | alk | N/A | —NH₂ | CD38 |
| 21 | Daratumumab | L5 | 153 | N/A | N/A | —NH₂ | CD38 |
| 22 | Daratumumab | L5 | 154 | alk | alk | —NH₂ | CD38 |
| 23 | Daratumumab | L5 | 155 | alk | alk | —NH₂ | CD38 |
| 24 | Daratumumab | L5 | 156 | alk | alk | —NH₂ | CD38 |
| 25 | Daratumumab | L5 | 157 | alk | alk | —NH₂ | CD38 |
| 26 | Daratumumab | L5 | 158 | alk | alk | —NH₂ | CD38 |
| 27 | Daratumumab | L5 | 159 | alk | alk | —NH₂ | CD38 |
| 28 | Daratumumab | L5 | 160 | alk | N/A | —NH₂ | CD38 |
| 29 | Daratumumab | L5 | 161 | alk | N/A | —NH₂ | CD38 |
| 30 | Daratumumab | L5 | 162 | alk | N/A | —NH₂ | CD38 |
| 31 | Daratumumab | L5 | 163 | alk | N/A | —NH₂ | CD38 |
| 32 | Daratumumab | L5 | 164 | alk | N/A | —NH₂ | CD38 |
| 33 | Daratumumab | L5 | 165 | alk | N/A | —NH₂ | CD38 |
| 34 | Daratumumab | L5 | 166 | alk | N/A | —NH₂ | CD38 |
| 35 | Daratumumab | L5 | 167 | N/A | N/A | —NH₂ | CD38 |
| 36 | Daratumumab | L5 | 168 | N/A | N/A | —NH₂ | CD38 |
| 37 | Daratumumab | L5 | 169 | N/A | N/A | —NH₂ | CD38 |
| 38 | Daratumumab | L5 | 170 | N/A | N/A | —NH₂ | CD38 |
| 39 | Daratumumab | L5 | 171 | N/A | N/A | —NH₂ | CD38 |
| 40 | Daratumumab | L5 | 172 | N/A | N/A | —NH₂ | CD38 |
| 52 | Mirvetuximab | L16 | 2 | alk | alk | —NH₂ | Folate |
| | | | | | | | Receptor |
| | | | | | | | Alpha |
| 53 | Mirvetuximab | L15 | 2 | alk | alk | —NH₂ | Folate |
| | | | | | | | Receptor |
| | | | | | | | Alpha |
| 84 | Mirvetuximab | L16 | 142 | alk | N/A | —NH₂ | Folate |
| | | | | | | | Receptor |
| | | | | | | | Alpha |
| 85 | Mirvetuximab | L16 | 154 | alk | alk | —NH₂ | Folate |
| | | | | | | | Receptor |
| | | | | | | | Alpha |
| 86 | Mirvetuximab | L16 | 160 | alk | N/A | —NH₂ | Folate |
| | | | | | | | Receptor |
| | | | | | | | Alpha |
| 87 | Mirvetuximab | L16 | 163 | alk | N/A | —NH₂ | Folate |
| | | | | | | | Receptor |
| | | | | | | | Alpha |
| 88 | Mirvetuximab | L16 | 165 | alk | N/A | —NH₂ | Folate |
| | | | | | | | Receptor |
| | | | | | | | Alpha |
| 110 | Daratumumab | L16 | 2 | alk | alk | —NH₂ | CD38 |
| 111 | Daratumumab | L16 | 142 | alk | N/A | —NH₂ | CD38 |
| 112 | Daratumumab | L16 | 154 | alk | alk | —NH₂ | CD38 |
| 113 | Daratumumab | L16 | 160 | alk | N/A | —NH₂ | CD38 |
| 114 | Daratumumab | L16 | 163 | alk | N/A | —NH₂ | CD38 |
| 115 | Daratumumab | L16 | 165 | alk | N/A | —NH₂ | CD38 |
| 146 | Isatuximab | L5 | 2 | alk | alk | —NH₂ | CD38 |
| 147 | Isatuximab | L27 | 2 | alk | alk | —NH₂ | CD38 |
| 148 | Isatuximab | L28 | 2 | alk | alk | —NH₂ | CD38 |
| 149 | Isatuximab | L29 | 2 | alk | alk | —NH₂ | CD38 |
| 150 | Isatuximab | L30 | 2 | alk | alk | —NH₂ | CD38 |
| 151 | Isatuximab | L9 | 15 | N/A | N/A | —NH₂ | CD38 |
| 152 | Isatuximab | L9 | 8 | N/A | N/A | —NH₂ | CD38 |
| 153 | Isatuximab | L9 | 7 | N/A | N/A | —NH₂ | CD38 |
| 154 | Isatuximab | L9 | 15 | N/A | N/A | —NH₂ | CD38 |
| 155 | Isatuximab | L9 | 8 | N/A | N/A | —NH₂ | CD38 |
| 156 | Isatuximab | L9 | 7 | N/A | N/A | —NH₂ | CD38 |
| 157 | Daratumumab | L24 | 2 | alk | alk | —NH₂ | CD38 |
| 158 | Daratumumab | L25 | 2 | alk | alk | —NH₂ | CD38 |
| 159 | Daratumumab | L26 | 2 | alk | alk | —NH₂ | CD38 |
| 160 | Daratumumab | L27 | 2 | alk | alk | —NH₂ | CD38 |
| 161 | Daratumumab | L28 | 2 | alk | alk | —NH₂ | CD38 |
| 162 | Daratumumab | L29 | 2 | alk | alk | —NH₂ | CD38 |
| 163 | Daratumumab | L30 | 2 | alk | alk | —NH₂ | CD38 |

### Promoting Internalization of Small Molecules

As described herein, an antibody or antigen-binding fragment thereof may be conjugated to a promoter peptide, forming a PAC, which may be further conjugated to an additional active agent (e.g., the antibody or antigen-binding fragment thereof is an antibody-drug conjugate (ADC)). In such embodiments, the PAC promotes internalization of the active agent into a cell, instead of or in addition to the antibody or antigen-binding fragment thereof.

In certain embodiments, the additional active agent is a small molecule (e.g., a small molecule cytotoxic agent). In certain embodiments, the PAC promotes internalization of the antibody or antigen-binding fragment thereof and the small molecule (e.g., the small molecule cytotoxic agent).

In some embodiments, the small molecule to be delivered (e.g., the small molecule cytotoxic agent to be delivered) is conjugated to the antibody or antigen-binding fragment thereof, the promoter peptide, or the linker of a PAC.

Provided herein are methods of promoting uptake (i.e., internalization) of a small molecule (e.g., a small molecule cytotoxic agent) into a cell, the methods comprising contacting the cell with a peptide-antibody conjugate (PAC), wherein the PAC comprises a promoter peptide conjugated to an antibody-drug conjugate (ADC); and wherein ADC comprises the small molecule conjugated to an antibody or antigen-binding fragment thereof.

In certain embodiments, provided herein are methods of promoting uptake (i.e., internalization) of a small molecule (e.g., a small molecule cytotoxic agent) into a cell, the methods comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a stapled peptide.

In certain embodiments, provided herein are methods of promoting uptake (i.e., internalization) of a small molecule (e.g., a small molecule cytotoxic agent) into a cell, the methods comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a Magainin peptide (e.g., a stapled or unstapled Magainin peptide, e.g., a stapled or unstapled peptide comprising SEQ ID NO: 1 or SEQ ID NO: 2).

In certain embodiments, provided herein are methods of promoting uptake (i.e., internalization) of a small molecule (e.g., a small molecule cytotoxic agent) into a cell, the methods comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a cationic promoter peptide (e.g., a promoter peptide comprising one of SEQ ID NOs: 5-19, e.g., any one of SEQ ID NOs: 7-18).

In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the small molecule is a small molecule cytotoxic agent. In certain embodiments, the cell is contacted in vivo (i.e., in a subject). In certain embodiments, the cell is contacted in vitro (e.g., in a cellular assay).

In certain embodiments, the percent uptake of the small molecule (e.g., the small molecule cytotoxic agent) into the cell is greater than 10% (e.g., greater than 20%, greater than 25%, greater than 30%, greater than 35%, greater than 40%, greater than 45%, greater than 50%, greater than 60%, greater than 65%, or greater than 70%, or greater than 75%, or greater than 80%, or greater than 85%, greater than 90%, or greater than 95%). In certain embodiments, the percent uptake of the small molecule (e.g., the small molecule cytotoxic agent) into the cell is greater than 25%. In certain embodiments, the percent uptake of the small molecule (e.g., the small molecule cytotoxic agent) into the cell is greater than 50%. In certain embodiments, the percent uptake of the small molecule (e.g., the small molecule cytotoxic agent) into the cell is greater than 75%.

In certain embodiments, uptake of the small molecule (e.g., the small molecule cytotoxic agent) into the cell is increased by at least 10% (e.g., by at least 25%, by at least 50%, by at least 75%, by at least 100%, by at least 200%, by at least 300%, by at least 400%, by at least 500%, by at least 600%, by at least 700%, by at least 800%, by at least 900%, by at least 1000%), relative to uptake of the small molecule (e.g., the small molecule cytotoxic agent) when the ADC is not conjugated to the promoter peptide. In certain embodiments, uptake of the small molecule (e.g., the small molecule cytotoxic agent) into the cell is increased by at least 25%, relative to uptake of the small molecule (e.g., the small molecule cytotoxic agent) when the ADC is not conjugated to the promoter peptide. In certain embodiments, uptake of the small molecule (e.g., the small molecule cytotoxic agent) into the cell is increased by at least 100%, relative to uptake of the small molecule (e.g., the small molecule cytotoxic agent) when the ADC is not conjugated to the promoter peptide. In certain embodiments, uptake of the small molecule (e.g., the small molecule cytotoxic agent) into the cell is increased by at least 200%, relative to uptake of the small molecule (e.g., the small molecule cytotoxic agent) when the ADC is not conjugated to the promoter peptide. In certain embodiments, uptake of the small molecule (e.g., the small molecule cytotoxic agent) into the cell is increased by at least 400%, relative to uptake of the small molecule (e.g., the small molecule cytotoxic agent) when the ADC is not conjugated to the promoter peptide. In certain embodiments, uptake of the small molecule (e.g., the small molecule cytotoxic agent) into the cell is increased by at least 600%, relative to uptake of the small molecule (e.g., the small molecule cytotoxic agent) when the ADC is not conjugated to the promoter peptide. In certain embodiments, uptake of the small molecule (e.g., the small molecule cytotoxic agent) into the cell is increased by at least 800%, relative to uptake of the small molecule (e.g., the small molecule cytotoxic agent) when the ADC is not conjugated to the promoter peptide. In certain embodiments, uptake of the small molecule (e.g., the small molecule cytotoxic agent) into the cell is increased by at least 1000%, relative to uptake of the small molecule (e.g., the small molecule cytotoxic agent) when the ADC is not conjugated to the promoter peptide.

In certain embodiments, the promoter peptide promotes uptake of the small molecule (e.g., the small molecule cytotoxic agent) into the cell, whereas the small molecule (e.g., the small molecule cytotoxic agent) is not internalized into the cell in a statistically significant amount when the ADC is not conjugated to the promoter peptide.

In certain embodiments, the ADC comprises an antibody directed against CD38, or an antigen-binding fragment thereof. In certain embodiments, the ADC comprises a monoclonal antibody (mAb) directed against CD38, or an antigen-binding fragment thereof. In certain embodiments, the ADC comprises daratumumab, or an antigen-binding fragment thereof. In certain embodiments, the ADC comprises daratumumab.

In certain embodiments, the ADC comprises an antibody directed against CD20, or an antigen-binding fragment thereof. In certain embodiments, the ADC comprises a monoclonal antibody (mAb) directed against CD20, or an antigen-binding fragment thereof. In certain embodiments, the ADC comprises obinutuzumab, or an antigen-binding fragment thereof. In certain embodiments, the ADC comprises obinutuzumab.

In certain embodiments, the ADC comprises an antibody directed against HER2, or an antigen-binding fragment thereof. In certain embodiments, the ADC comprises a monoclonal antibody (mAb) directed against HER2, or an antigen-binding fragment thereof. In certain embodiments, the ADC comprises trastuzumab, or an antigen-binding fragment thereof. In certain embodiments, the ADC comprises trastuzumab.

In certain embodiments, the ADC comprises an antibody directed against HER3, or an antigen-binding fragment thereof. In certain embodiments, the ADC comprises a monoclonal antibody (mAb) directed against HER3, or an antigen-binding fragment thereof. In certain embodiments, the ADC comprises patritumab, or an antigen-binding fragment thereof. In certain embodiments, the ADC comprises patritumab.

In certain embodiments, the ADC comprises an antibody directed against EGFR, or an antigen-binding fragment thereof. In certain embodiments, the ADC comprises a monoclonal antibody (mAb) directed against EGFR, or an antigen-binding fragment thereof. In certain embodiments, the ADC comprises cetuximab, or an antigen-binding fragment thereof. In certain embodiments, the ADC comprises cetuximab.

In certain embodiments, the ADC comprises an antibody directed against Folate Receptor Alpha, or an antigen-binding fragment thereof. In certain embodiments, the ADC comprises a monoclonal antibody (mAb) directed against Folate Receptor Alpha, or an antigen-binding fragment thereof. In certain embodiments, the ADC comprises mirvetuximab, or an antigen-binding fragment thereof. In certain embodiments, the ADC comprises mirvetuximab.

Promoter peptides are described herein. In certain embodiments, the promoter peptide is a stapled peptide (e.g., a singly stapled, doubly stapled, or stitched peptide). In certain embodiments, the promoter peptide is a stapled Magainin peptide. In certain embodiments, the promoter peptide is a stapled Magainin II peptide. In certain embodiments, the promoter peptide is a stapled peptide comprising SEQ ID NO: 1 (wherein X¹ and X² are connected via a crosslink, and X³ and X⁴ are connected via a crosslink). In certain embodiments, the promoter peptide is a stapled peptide comprising SEQ ID NO: 2 (wherein X¹ and X² are connected via a crosslink, and X³ and X⁴ are connected via a crosslink). In certain embodiments, the promoter peptide comprises any one of SEQ ID NOs: 1-21, 27-135, and 142-174 (wherein X¹ and X² are connected via a crosslink, and X³ and X⁴ are connected via a crosslink, where applicable). In certain embodiments, the promoter peptide comprises any one of SEQ ID NOs: 142-174 (wherein X¹ and X² are connected via a crosslink, and X³ and X⁴ are connected via a crosslink, where applicable).

In certain embodiments, the ADC comprises an anti-cancer agent (e.g., a small-molecule anti-cancer agent), conjugated to the antibody or antigen-binding fragment thereof. The small molecule anti-cancer agent may be a small molecule cytotoxic agent. Exemplary anti-cancer agents include, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™ AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), nilotinib (TASIGNA®), sorafenib (NEXAVAR®), everolimus (AFINITOR®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the small molecule cytotoxic agent is selected from auristatins, maytansinoids, camptothecins (e.g., deruxtecan, SN38), tubulysins, calicheamycins, duocarymycins, pyrrolobenzodiazepines (PBD) dimers, amatoxins, and immunotoxins. In certain embodiments, the small molecule cytotoxic agent is a maytansinoid (e.g., maytansine, DM1, DM4).

In certain embodiments, the small molecule is a protein-protein interaction (PPI) inhibitor. In certain embodiments, the small molecule is an inhibitor of a Bcl-2 family member protein. In certain embodiments, the small molecule is an MCL-1 inhibitor (e.g., MIK665).

Table 3 below provides examples of antibody-drug conjugates (ADCs) (including generic name, trade name, and exemplary use(s)). Any of these ADCs may be conjugated to a promoter peptide to form a PAC described herein. Therapeutic applications of the ADCs listed below are not limited to the particular known target antigen(s) and exemplary use(s) provided.

TABLE 3
Examples of Antibody-Drug Conjugates (ADCs)

| | | Trade |
|---|---|---|
| Generic Name | Exemplary Use(s) | Name |
| Gemtuzumab | acute myelogenous leukemia (AML) | Mylotarg |
| ozogamicin | | |
| Brentuximab | Hodgkin lymphoma (HL) and systemic | Adcetris |
| vedotin | anaplastic large-cell lymphoma (ALCL) | |
| Trastuzumab | breast cancer (e.g., HER2-positive | Kadcyla |
| emtansine | metastatic breast cancer) | |
| Inotuzumab | acute lymphoblastic leukemia (ALL) | Besponsa |
| ozogamicin | | |
| Polatuzumab | diffuse large B-cell lymphoma | Polivy |
| vedotin | (DLBCL) | |
| Enfortumab vedotin | urothelial cancer | Padcev |
| Trastuzumab | breast cancer (e.g., HER2-positive | Enhertu |
| deruxtecan | metastatic breast cancer) | |
| Sacituzumab | breast cancer (e.g., metastatic triple- | Trodelvy |
| govitecan | negative breast cancer) | |
| Belantamab | multiple myeloma | Blenrep |
| mafodotin | | |
| Moxetumomab | hairy cell leukemia (HCL) | Lumoxiti |
| pasudotox | | |
| Loncastuximab | large B-cell lymphoma (including | Zynlonta |
| tesirine | diffuse large B-cell lymphoma | |
| | (DLBCL), DLBCL arising from low- | |
| | grade lymphoma, and high-grade | |
| | B-cell lymphoma) | |
| Tisotumab | cervical | Tivdak |
| vedotin-tftv | | |
| Patritumab | Non-small cell lung cancer (NSCLC), | |
| deruxtecan | breast cancer | |
| Mirvetuximab | Fallopian tube cancer, primary | Elahere |
| soravtansine | peritoneal cancer | |
| lifastuzumab | Non-small cell lung cancer (NSCLC), | |
| vedotin | ovarian cancer | |

Other non-limiting examples of small molecules (e.g., small molecule cytotoxic agents (“cytotoxins”)) are provided in Table 3A below. In certain embodiments, the small molecule cytotoxic agent is MMAF. In certain embodiments, the small molecule cytotoxic agent is MMAE. In certain embodiments, the small molecule cytotoxic agent is DM1. In certain embodiments, the small molecule cytotoxic agent is Exatecan. In certain embodiments, the small molecule cytotoxic agent is DX-8951 (e.g., as Deruxtecan). In certain embodiments, the small molecule cytotoxic agent is SG3199 (e.g., as Tesirine).

In certain embodiments, the small molecule is an MCL-1 inhibitor such as MIK665.

TABLE 3A
Examples of Small Molecules

| Name and | | |
|---|---|---|
| Function | Abbreviation | Structure |
| Monomethyl auristatin F (cytotoxin) | MMAF | 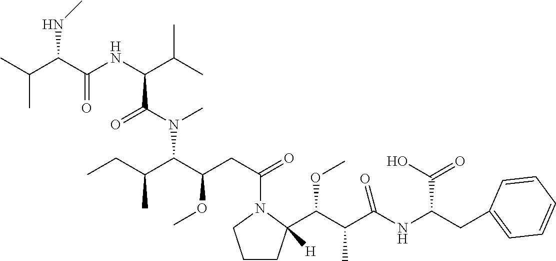 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C] |
| Monomethyl auristatin E (cytotoxin) | MMAE | 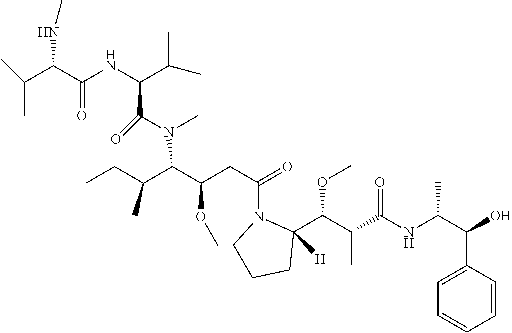 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C] |
| Mertansine (cytotoxin) | DM1 | 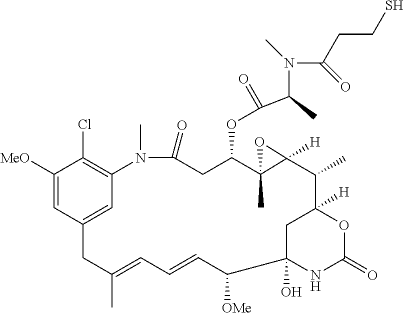 |
| Exatecan (cytotoxin) | Exa | 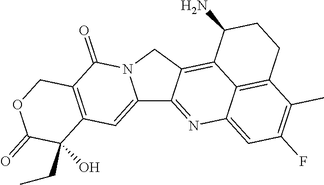 [SMILES: CC[C@@]1(O)C(=O)OCc2c1cc1n(c2=O)Cc2c-1nc1cc(F)c(C)c3c1c2[C@@H](N)CC3] |
| DX-8951 (cytotoxin) | DX-8951 | 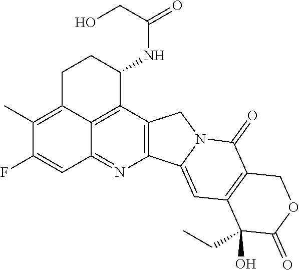 [SMILES: CC[C@@]1(O)C(=O)OCc2c1cc1n(c2=O)Cc2c-1nc1cc(F)c(C)c3c1c2[C@@H](NC(=O)CO)CC3] |
| MIK665 (MCL-1 inhibitor) | MIK665 | 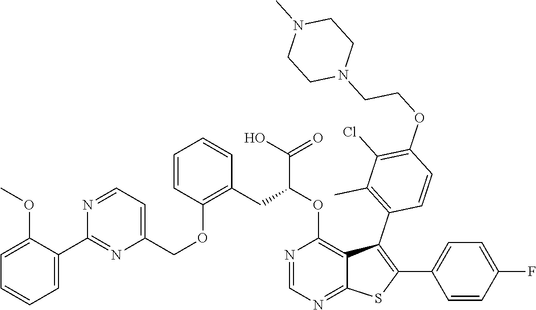 |
| SG3199 (cytotoxin) | SG3199 | 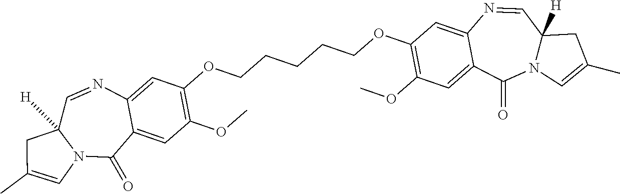 |

As described, provided herein are peptide antibody conjugates (PACs) comprising an antibody or antigen-binding fragment thereof conjugated to a promoter peptide, wherein the PAC is further conjugated to a small molecule cytotoxic agent.

In certain embodiments, the small molecule cytotoxic agent (“cytotoxin”) can be conjugated to the PAC via a linker. As described, the cytotoxin can be conjugated to the antibody or antigen-binding fragment thereof of the PAC and/or conjugated to a linker of the PAC. Table 3B below provides non-limiting examples of reagents comprising a linker and a cytotoxin (“Linker-Cytotoxin”) used to conjugate the cytotoxin to a PAC described herein. In the case of T1-T4 and T6-T12, the maleimide of the Linker-Cytotoxin reacts with and is conjugated to a thiol (e.g., cysteine) on the PAC. In the case of T5, the N-hydroxysuccinimidyl ester of the Linker-Cytotoxin reacts with and is conjugated to an amine (e.g., lysine) on the PAC. Note that in the case of T9, the exemplified small molecule is an MCL-1 inhibitor rather than a cytotoxin.

TABLE 3B
Examples of Linker-Cytotoxin Reagents

| Linker- | Linker- | | |
|---|---|---|---|
| Cytotoxin | Cytotoxin | Linker-Cytotoxin | |
| # | Name | Structure* | Cytotoxin |
| T1 | MC-VCit- PAB-MMAF | 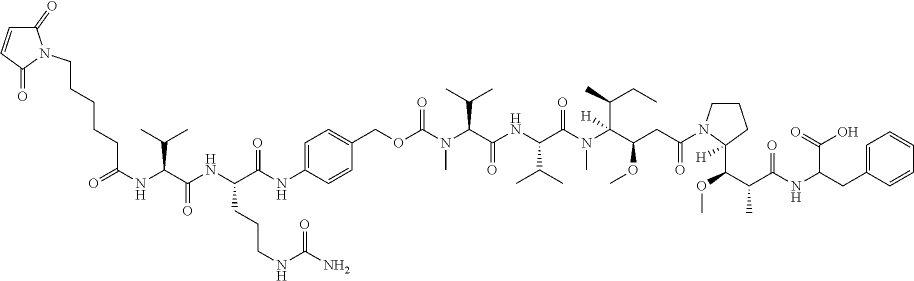 [SMILES: CC[C@H](C)[C@@H]([C@@H](CC(=O)N1CCC[C@H]1[C@H](OC)[C@@H](C)C(=O)NC(Cc1ccccc1)C(=O)O)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C] | MMAF |
| T2 | MC-VCit- PAB-MMAE | 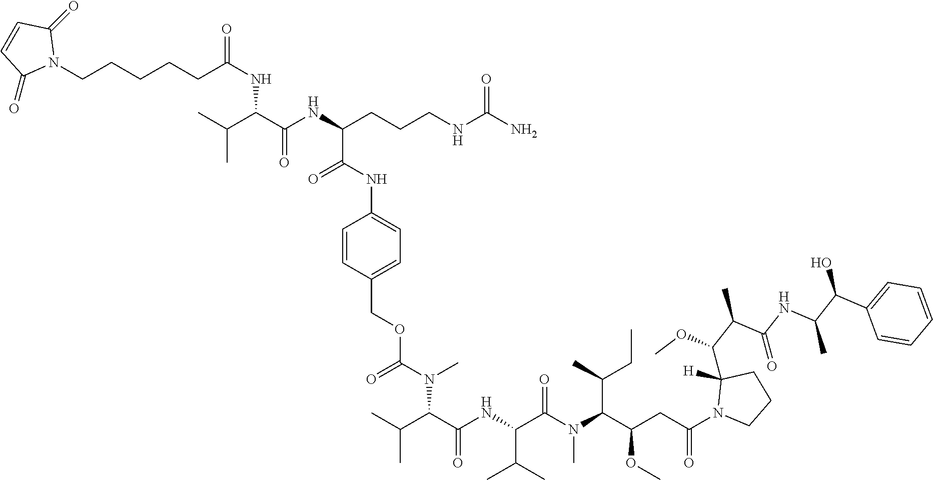 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C] | MMAE |
| T3 | MC-MMAF | 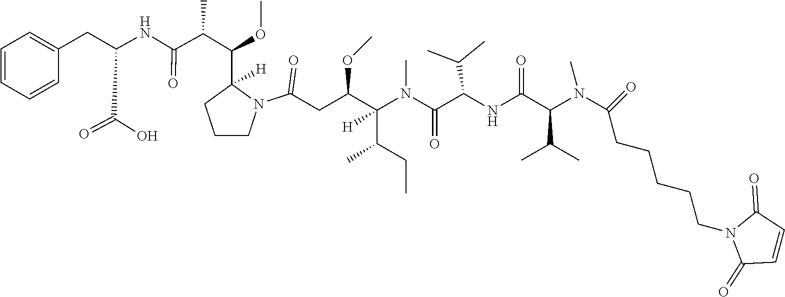 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C] | MMAF |
| T4 | MC- MAB(GlcA)- MMAE | 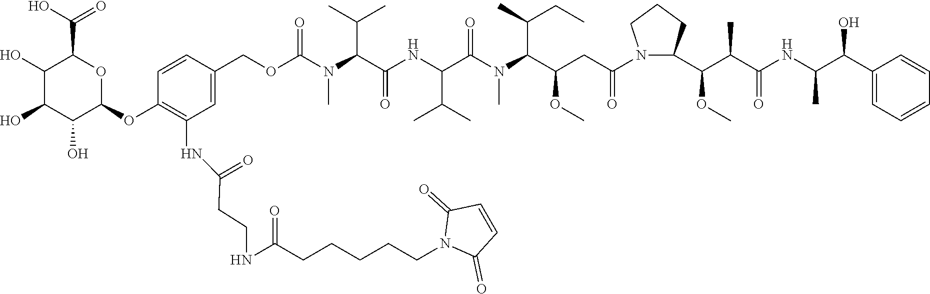 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)C(NC(=O)[C@H](C(C)C)N(C)C(=O)OCc1ccc(O[C@@H]2O[C@H](C(=O)O)C(O)[C@H](O)[C@H]2O)c(NC(=O)CCNC(=O)CCCCCN2C(=O)C=CC2=O)c1)C(C)C] | MMAE |
| T5 | SMCC-DM1 | 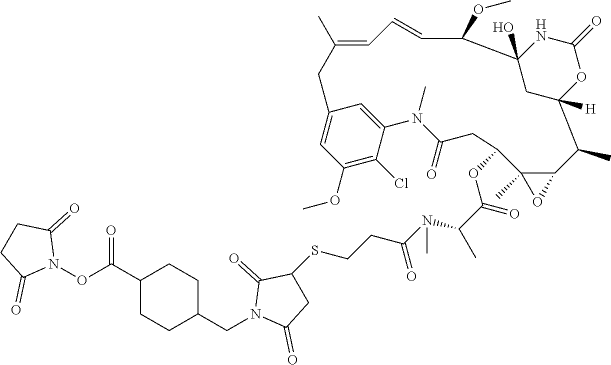 [SMILES: COc1cc2cc(c1Cl)N(C)C(=O)C[C@H](OC(=O)[C@H](C)N(C)C(=O)CCSC1CC(=O)N(CC3CCC(C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2] | DM1 |
| T6 | MC-VC- PAB- Exatecan | 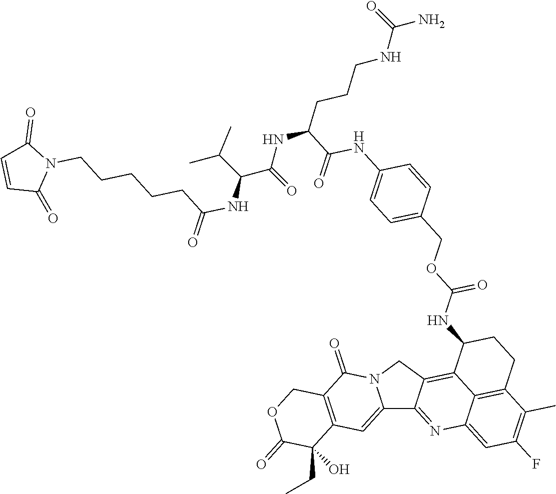 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCCCCN2C(=O)C=CC2=O)C(C)C)cc1)CC3] | Exatecan (Exa) |
| T7 | Mal-PEG8- VA-PAB- Exatecan | 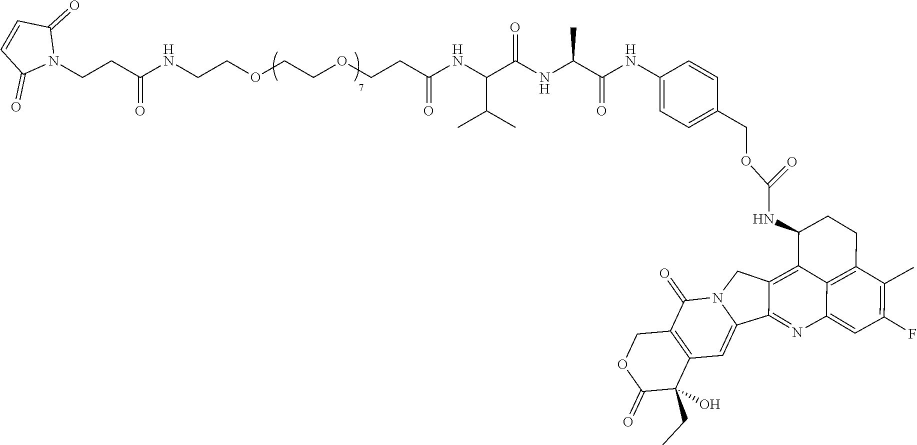 | Exatecan (Exa) |
| T8 | Mal- MAB(GlcA)- Exatecan | 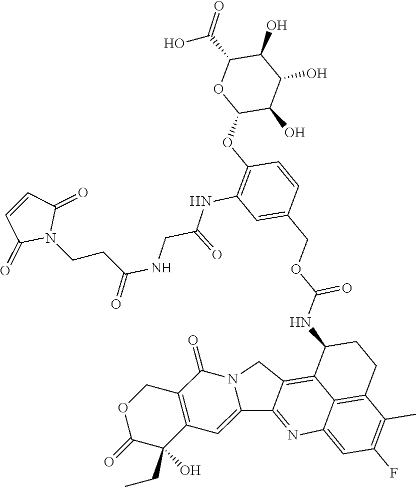 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CCN2C(=O)C=CC2=O)c1)CC3] | Exatecan (Exa) |
| T9** | MC-VCit- PAB- MIK665 | 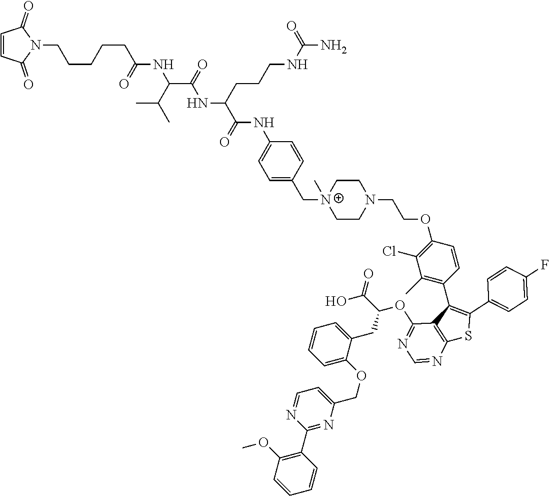 | MIK665 (MCL-1 inhibitor) |
| T10 | Deruxtecan | 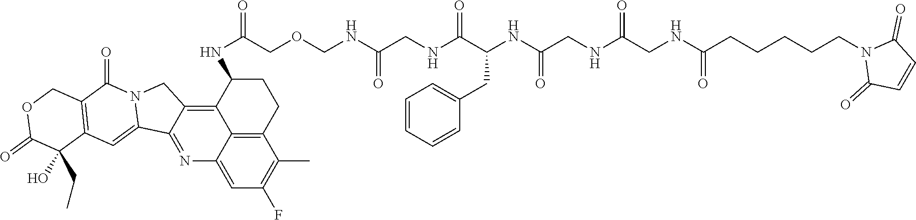 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@@H](Cc1ccccc1)NC(=O)CNC(=O)CNC(=O)CCCCCN1C(=O)C=CC1=O)CC3] | DX-8951 |
| T11 | Tesirine | 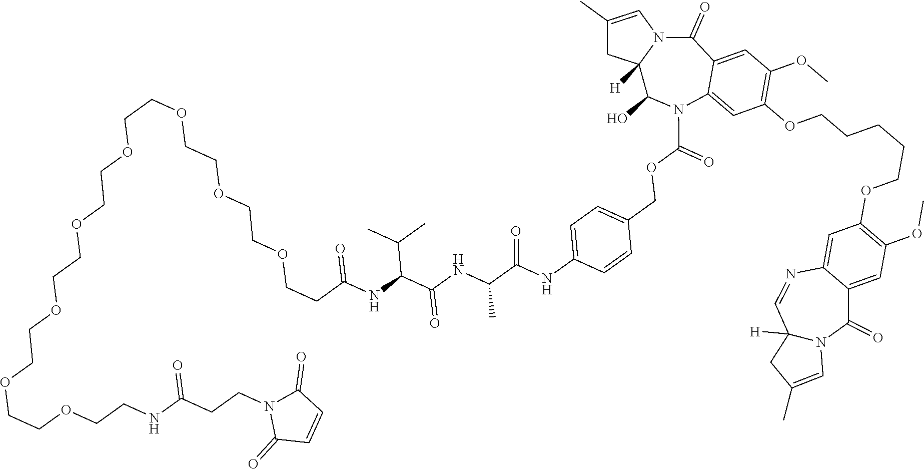 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)cc1)N=CC1CC(C)=CN1C2=O] | SG3199 |
| T12 | MC-GGFG- Exatecan | 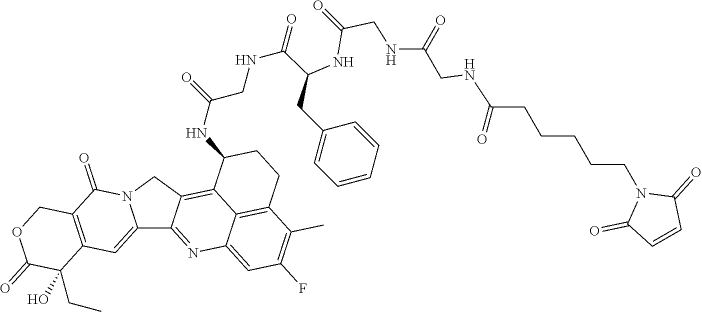 [SMILES: CC[C@@]1(O)C(=O)OCc2c1cc1n(c2=O)Cc2c-1nc1cc(F)c(C)c3c1c2[C@@H](NC(=O)CNC(=O)[C@H](Cc1ccccc1)NC(=O)CNC(=O)CNC(=O)CCCCCN1C(=O)C=CC1=O)CC3] | Exatecan (Exa) |

*In the case of T1-T4 and T6-T12, the maleimide of the linker-cytotoxin is reacts with and is conjugated to a thiol (e.g., cysteine) on the PAC. In the case of T5, the N-hydroxysuccinimidyl ester of the Linker-Cytotoxin reacts with and is conjugated to an amine (e.g., lysine) on the PAC.
**In the case of T9, the exemplified small molecule is an MCL-1 inhibitor rather than a cytotoxin.

As described herein, in certain embodiments, the cytotoxin is conjugated to the antibody or antigen-binding fragment thereof of the PAC. Scheme 1 below shows a schematic of the embodiments wherein the cytotoxin is conjugated to the PAC via the antibody or antigen-binding fragment thereof (“AB”). Table 3C below provides examples of PACs wherein the cytotoxin is conjugated to the antibody (or antigen-binding fragment thereof) component of the PAC.

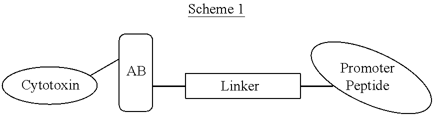

TABLE 3C
Examples of PACs with Cytotoxin Conjugated to Antibody Component

| | | | Promoter | | | Promoter | | | |
|---|---|---|---|---|---|---|---|---|---|
| | | | Peptide | X¹-X² | X³-X⁴ | Peptide | | | |
| PAC | | | SEQ ID | Cross | Cross | C- | Cyto- | Linker- | |
| # | Antibody | Linker | NO: | link | link | Terminus | toxin | Cytotoxin* | Target |
| 41 | Daratumumab | L5 | 2 | alk | alk | —NH₂ | MMAF | T1 | CD38 |
| 42 | Daratumumab | L5 | 142 | alk | N/A | —NH₂ | MMAF | T1 | CD38 |
| 43 | Daratumumab | L5 | 154 | alk | alk | —NH₂ | MMAF | T1 | CD38 |
| 44 | Daratumumab | L5 | 160 | alk | N/A | —NH₂ | MMAF | T1 | CD38 |
| 45 | Daratumumab | L5 | 165 | alk | N/A | —NH₂ | MMAF | T1 | CD38 |
| 46 | Patritumab | L5 | 2 | alk | alk | —NH₂ | MMAF | T1 | HER3 |
| 47 | Patritumab | L9 | 15 | N/A | N/A | —NH₂ | MMAF | T1 | HER3 |
| 48 | Patritumab | L9 | 8 | N/A | N/A | —NH₂ | MMAF | T1 | HER3 |
| 49 | Patritumab | L5 | 160 | alk | N/A | —NH₂ | MMAF | T1 | HER3 |
| 50 | Patritumab | L5 | 165 | alk | N/A | —NH₂ | MMAF | T1 | HER3 |
| 54 | Mirvetuximab | L16 | 2 | alk | alk | —NH₂ | MMAE | T2 | Folate |
| | | | | | | | | | Receptor |
| | | | | | | | | | Alpha |
| 89 | Mirvetuximab | L16 | 2 | alk | alk | —NH₂ | MMAE | T4 | Folate |
| | | | | | | | | | Receptor |
| | | | | | | | | | Alpha |
| 90 | Mirvetuximab | L16 | 142 | alk | N/A | —NH₂ | MMAE | T4 | Folate |
| | | | | | | | | | Receptor |
| | | | | | | | | | Alpha |
| 91 | Mirvetuximab | L16 | 154 | alk | alk | —NH₂ | MMAE | T4 | Folate |
| | | | | | | | | | Receptor |
| | | | | | | | | | Alpha |
| 92 | Mirvetuximab | L16 | 160 | alk | N/A | —NH₂ | MMAE | T4 | Folate |
| | | | | | | | | | Receptor |
| | | | | | | | | | Alpha |
| 93 | Mirvetuximab | L16 | 163 | alk | N/A | —NH₂ | MMAE | T4 | Folate |
| | | | | | | | | | Receptor |
| | | | | | | | | | Alpha |
| 94 | Mirvetuximab | L16 | 165 | alk | N/A | —NH₂ | MMAE | T4 | Folate |
| | | | | | | | | | Receptor |
| | | | | | | | | | Alpha |
| 116 | Daratumumab | L16 | 2 | alk | alk | —NH₂ | MMAE | T4 | CD38 |
| 117 | Daratumumab | L16 | 142 | alk | N/A | —NH₂ | MMAE | T4 | CD38 |
| 118 | Daratumumab | L16 | 154 | alk | alk | —NH₂ | MMAE | T4 | CD38 |
| 119 | Daratumumab | L16 | 160 | alk | N/A | —NH₂ | MMAE | T4 | CD38 |
| 120 | Daratumumab | L16 | 163 | alk | N/A | —NH₂ | MMAE | T4 | CD38 |
| 121 | Daratumumab | L16 | 165 | alk | N/A | —NH₂ | MMAE | T4 | CD38 |
| 144 | Daratumumab | L5 | 2 | alk | alk | —NH₂ | DM1 | T5 | CD38 |
| 145 | Daratumumab | L11 | 8 | N/A | N/A | —NH₂ | DM1 | T5 | CD38 |
| 170 | Vadastuximab | L5 | 2 | alk | alk | —NH₂ | Der | T10 | CD33 |
| | ** | | | | | | | | |

*For PACs 41-50, the Linker-Cytotoxin is conjugated to a lysine of the antibody via an AJICAP reagent. See, e.g., Yamada et al. “AJICAP: Affinity Peptide Mediated Regiodivergent Functionalization of Native Antibodies” Angew. Chem. Int. Ed. 2019, vol. 58, 5592-5597. For the remaining PACs in Table 3C, the Linker-Cytotoxin is conjugated to antibody cysteines.
** Vadastuximab S239C.

As described herein, in other embodiments, the cytotoxin is conjugated to the PAC via a linker between the antibody (or antigen-binding fragment thereof) and the promoter peptide. Scheme 2 below shows a schematic of the embodiments wherein the cytotoxin is conjugated to the linker of the PAC (“AB”=antibody or antigen-binding fragment thereof). In such embodiments, one or more cytotoxins may also be conjugated to the antibody or antigen-binding fragment thereof. Table 3D below provides examples of PACs wherein the cytotoxin is conjugated to the linker between the antibody and the promoter peptide.

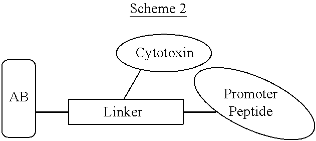

TABLE 3D
Examples of PACs with Cytotoxin Conjugated to the Linker*

| | | | Promoter | | | Promoter | | | |
|---|---|---|---|---|---|---|---|---|---|
| | | | Peptide | X¹-X² | X³-X⁴ | Peptide | | | |
| | | | SEQ ID | Cross- | Cross- | C- | | Linker- | |
| PAC# | Antibody | Linker | NO: | link | link | Terminus | Cytotoxin | Cytotoxin | Target |
| 51 | Patritumab | L15 | 2 | alk | alk | —NH₂ | MMAE | T2 | HER3 |
| 55 | Mirvetuximab | L15 | 2 | alk | alk | —NH₂ | MMAE | T2 | Folate |
| | | | | | | | | | Receptor |
| | | | | | | | | | Alpha |
| 56 | Daratumumab | L15 | 142 | alk | N/A | —NH₂ | MMAE | T2 | CD38 |
| 57 | Daratumumab | L15 | 154 | alk | alk | —NH₂ | MMAE | T2 | CD38 |
| 58 | Daratumumab | L15 | 160 | alk | N/A | —NH₂ | MMAE | T2 | CD38 |
| 59 | Daratumumab | L15 | 163 | alk | N/A | —NH₂ | MMAE | T2 | CD38 |
| 60 | Daratumumab | L15 | 165 | alk | N/A | —NH₂ | MMAE | T2 | CD38 |
| 61 | Daratumumab | L15 | 2 | alk | alk | —NH₂ | MMAE | T2 | CD38 |
| 62 | Daratumumab | L15 | 142 | alk | N/A | —NH₂ | MMAF | T3 | CD38 |
| 63 | Daratumumab | L15 | 154 | alk | alk | —NH₂ | MMAF | T3 | CD38 |
| 64 | Daratumumab | L15 | 160 | alk | N/A | —NH₂ | MMAF | T3 | CD38 |
| 65 | Daratumumab | L15 | 163 | alk | N/A | —NH₂ | MMAF | T3 | CD38 |
| 66 | Daratumumab | L15 | 165 | alk | N/A | —NH₂ | MMAF | T3 | CD38 |
| 67 | Daratumumab | L15 | 2 | alk | alk | —NH₂ | MMAF | T3 | CD38 |
| 68 | Daratumumab | L15 | 142 | alk | N/A | —NH₂ | MMAF | T1 | CD38 |
| 69 | Daratumumab | L15 | 154 | alk | alk | —NH₂ | MMAF | T1 | CD38 |
| 70 | Daratumumab | L15 | 160 | alk | N/A | —NH₂ | MMAF | TI | CD38 |
| 71 | Daratumumab | L15 | 163 | alk | N/A | —NH₂ | MMAF | T1 | CD38 |
| 72 | Daratumumab | L15 | 165 | alk | N/A | —NH₂ | MMAF | T1 | CD38 |
| 73 | Daratumumab | L15 | 2 | alk | alk | —NH₂ | MMAF | T1 | CD38 |
| 74 | Daratumumab | L15 | 142 | alk | N/A | —NH₂ | MMAE | T4 | CD38 |
| 75 | Daratumumab | L15 | 154 | alk | alk | —NH₂ | MMAE | T4 | CD38 |
| 76 | Daratumumab | L15 | 160 | alk | N/A | —NH₂ | MMAE | T4 | CD38 |
| 77 | Daratumumab | L15 | 163 | alk | N/A | —NH₂ | MMAE | T4 | CD38 |
| 78 | Daratumumab | L15 | 165 | alk | N/A | —NH₂ | MMAE | T4 | CD38 |
| 79 | Daratumumab | L15 | 2 | alk | alk | —NH₂ | MMAE | T4 | CD38 |
| 80 | Patritumab | L15 | 2 | alk | alk | —NH₂ | MMAE | T4 | HER3 |
| 81 | Mirvetuximab | L15 | 2 | alk | alk | —NH₂ | MMAE | T4 | Folate |
| | | | | | | | | | Receptor |
| | | | | | | | | | Alpha |
| 82 | Patritumab | L15 | 160 | alk | N/A | —NH₂ | MMAE | T4 | HER3 |
| 83 | Patritumab | L15 | 160 | alk | N/A | —NH₂ | MMAE | T2 | HER3 |
| 95 | Daratumumab | L17 | 160 | alk | N/A | —NH₂ | MMAE | T2 | CD38 |
| 96 | Daratumumab | L17 | 160 | alk | N/A | —NH₂ | MMAE | T4 | CD38 |
| 97 | Daratumumab | L17 | 160 | alk | N/A | —NH₂ | MMAE | T1 | CD38 |
| 98 | Daratumumab | L18 | 2 | alk | alk | —NH₂ | MMAF | T3 | CD38 |
| 99 | Daratumumab | L19 | 2 | alk | alk | —NH₂ | MMAF | T3 | CD38 |
| 100 | Daratumumab | L15 | 2 | alk | alk | —CO₂H | MMAE | T2 | CD38 |
| 101 | Daratumumab | L15 | 173 | alk | alk | —CO₂H | MMAE | T2 | CD38 |
| 102 | Daratumumab | L15 | 142 | alk | N/A | —CO₂H | MMAE | T2 | CD38 |
| 103 | Daratumumab | L15 | 174 | alk | N/A | —CO₂H | MMAE | T2 | CD38 |
| 104 | Daratumumab | L15 | 160 | alk | N/A | —CO₂H | MMAE | T2 | CD38 |
| 105 | Daratumumab | L15 | 2 | alk | alk | —CO₂H | MMAE | T4 | CD38 |
| 106 | Daratumumab | L15 | 173 | alk | alk | —CO₂H | MMAE | T4 | CD38 |
| 107 | Daratumumab | L15 | 142 | alk | N/A | —CO₂H | MMAE | T4 | CD38 |
| 108 | Daratumumab | L15 | 174 | alk | N/A | —CO₂H | MMAE | T4 | CD38 |
| 109 | Daratumumab | L15 | 160 | alk | N/A | —CO₂H | MMAE | T4 | CD38 |
| 122 | Daratumumab | L20 | 160 | alk | N/A | —NH₂ | MMAE | T4 | CD38 |
| 123 | Daratumumab | L21 | 160 | alk | N/A | —NH₂ | MMAE | T4 | CD38 |
| 124 | Daratumumab | L22 | 160 | alk | N/A | —NH₂ | MMAE | T4 | CD38 |
| 125 | Daratumumab | L22 | 2 | alk | alk | —NH₂ | MMAE | T4 | CD38 |
| 126 | Daratumumab | L17 | 2 | alk | alk | —NH₂ | MMAE | T4 | CD38 |
| 127 | Daratumumab | L17 | 142 | alk | N/A | —NH₂ | MMAE | T4 | CD38 |
| 128 | Daratumumab | L17 | 154 | alk | alk | —NH₂ | MMAE | T4 | CD38 |
| 129 | Daratumumab | L17 | 160 | alk | N/A | —NH₂ | MMAE | T4 | CD38 |
| 130 | Daratumumab | L17 | 163 | alk | N/A | —NH₂ | MMAE | T4 | CD38 |
| 131 | Daratumumab | L23 | 2 | alk | alk | —NH₂ | MMAE | T4 | CD38 |
| 132 | Daratumumab | L23 | 142 | alk | N/A | —NH₂ | MMAE | T4 | CD38 |
| 133 | Daratumumab | L23 | 154 | alk | alk | —NH₂ | MMAE | T4 | CD38 |
| 134 | Daratumumab | L23 | 160 | alk | N/A | —NH₂ | MMAE | T4 | CD38 |
| 135 | Daratumumab | L23 | 163 | alk | N/A | —NH₂ | MMAE | T4 | CD38 |
| 136 | Daratumumab | L23 | 165 | alk | N/A | —NH₂ | MMAE | T4 | CD38 |
| 137 | Daratumumab | L17 | 2 | alk | alk | —NH₂ | MMAE | T2 | CD38 |
| 138 | Daratumumab | L17 | 142 | alk | N/A | —NH₂ | MMAE | T2 | CD38 |
| 139 | Daratumumab | L17 | 154 | alk | alk | —NH₂ | MMAE | T2 | CD38 |
| 140 | Daratumumab | L17 | 163 | alk | N/A | —NH₂ | MMAE | T2 | CD38 |
| 141 | Daratumumab | L17 | 165 | alk | N/A | —NH₂ | MMAE | T2 | CD38 |
| 142 | Daratumumab | L20 | 160 | alk | N/A | —NH₂ | MMAE | T2 | CD38 |
| 143 | Daratumumab | L21 | 160 | alk | N/A | —NH₂ | MMAE | T2 | CD38 |
| 164 | Daratumumab | L15 | 2 | alk | alk | —NH₂ | Exa | T6 | CD38 |
| 165 | Daratumumab | L15 | 2 | alk | alk | —NH₂ | Exa | T7 | CD38 |
| 166 | Daratumumab | L15 | 2 | alk | alk | —NH₂ | Exa | T8 | CD38 |
| 167 | Daratumumab | L15 | 2 | alk | alk | —NH₂ | MIK665** | T9 | CD38 |
| 168 | Cetuximab | L15 | 2 | alk | alk | —NH₂ | MMAE | T2 | EGFR |
| 169 | Nimotuzumab | L15 | 2 | alk | alk | —NH₂ | MMAE | T2 | EGFR |
| 171 | Inebilizumab | L15 | 2 | alk | alk | —NH₂ | Tes | T11 | CD19 |
| 172 | Obinutuzumab | L15 | 2 | alk | alk | —NH₂ | Tes | T11 | CD20 |
| 173 | Tafasitamab | L15 | 2 | alk | alk | —NH₂ | Tes | T11 | CD19 |

*One or more Linker-Cytotoxins may also be conjugated to the antibody (e.g., via one or cysteines of the antibody)
**MIK665 is an MCL-1 inhibitor.

### Promoting Internalization of Target Proteins

Also provided herein are methods of promoting uptake (i.e., internalization) of a target protein (e.g., receptor) into a cell, the methods comprising contacting the cell with a conjugate, wherein the conjugate comprises a promoter peptide conjugated to an agent that binds the target protein.

In certain embodiments, provided herein are methods of promoting uptake (i.e., internalization) of a target protein (e.g., receptor) into a cell, the methods comprising contacting the cell with a conjugate, wherein the conjugate comprises a promoter peptide conjugated to an agent that binds the target protein; and wherein the promoter peptide is a stapled peptide.

In certain embodiments, provided herein are methods of promoting uptake (i.e., internalization) of a target protein (e.g., receptor) into a cell, the methods comprising contacting the cell with a conjugate, wherein the conjugate comprises a promoter peptide conjugated to an agent that binds the target protein; and wherein the promoter peptide is a Magainin peptide (e.g., a stapled or unstapled Magainin peptide, e.g., a stapled or unstapled peptide comprising SEQ ID NO: 1 or SEQ ID NO: 2).

In certain embodiments, provided herein are methods of promoting uptake (i.e., internalization) of a target protein (e.g., receptor) into a cell, the methods comprising contacting the cell with a conjugate, wherein the conjugate comprises a promoter peptide conjugated to an agent that binds the target protein; and wherein the promoter peptide is a cationic promoter peptide (e.g., a promoter peptide comprising one of SEQ ID NOs: 5-19, e.g., any one of SEQ ID NOs: 7-18).

In certain embodiments, the promoter peptide comprises any one of SEQ ID NOs: 1-21, 27-135, and 142-174. In certain embodiments, the promoter peptide comprises any one of SEQ ID NOs: 142-174.

In certain embodiments, the target protein is a protein on the surface of the cell. In certain embodiments, the target protein is a receptor. In certain embodiments, the receptor is selected from HER2, HER3, EGFR, GPCR, PCKS9, Nav1.7, CD33, CD30, CD32b, and PD1. In certain embodiments, the receptor is HER2. In certain embodiments, the receptor is EGFR. In certain embodiments, the target protein is a surface antigen. In certain embodiments, the surface antigen is selected from CD20, CD19, CEACAM5, EpCAM, Nectin-4, Trop-2, CD142, and CanAg. In certain embodiments, the surface antigen is CD20. In certain embodiments, the target protein is a surface enzyme, e.g., CD38. Other non-limiting examples of target proteins (e.g., receptors) are provided herein.

In certain embodiments, the conjugate is a PAC described herein, i.e., the agent that binds the target protein is an antibody or antigen-binding fragment thereof. In certain embodiments, the cell is contacted in vivo (i.e., in a subject). In certain embodiments, the cell is contacted in vitro (e.g., in a cellular assay).

In certain embodiments, the percent uptake of the target protein (e.g., receptor) into the cell is greater than 10% (e.g., greater than 20%, greater than 25%, greater than 30%, greater than 35%, greater than 40%, greater than 45%, greater than 50%, greater than 60%, greater than 65%, or greater than 70%, or greater than 75%, or greater than 80%, or greater than 85%, greater than 90%, or greater than 95%). In certain embodiments, the percent uptake of the target protein (e.g., receptor) into the cell is greater than 25%. In certain embodiments, the percent uptake of the target protein (e.g., receptor) into the cell is greater than 50%. In certain embodiments, the percent uptake of the target protein (e.g., receptor) into the cell is greater than 75%.

In certain embodiments, uptake of the target protein (e.g., receptor) into the cell is increased by at least 10% (e.g., by at least 25%, by at least 50%, by at least 75%, by at least 100%, by at least 200%, by at least 300%, by at least 400%, by at least 500%, by at least 600%, by at least 700%, by at least 800%, by at least 900%, by at least 1000%), relative to uptake of the target protein (e.g., receptor) when the agent that binds the target protein is not conjugated to the promoter peptide. In certain embodiments, uptake of the target protein (e.g., receptor) into the cell is increased by at least 25%, relative to uptake of the target protein (e.g., receptor) when the agent that binds the target protein is not conjugated to the promoter peptide. In certain embodiments, uptake of the target protein (e.g., receptor) into the cell is increased by at least 100%, relative to uptake of the target protein (e.g., receptor) when the agent that binds the target protein is not conjugated to the promoter peptide. In certain embodiments, uptake of the target protein (e.g., receptor) into the cell is increased by at least 200%, relative to uptake of the target protein (e.g., receptor) when the agent that binds the target protein is not conjugated to the promoter peptide. In certain embodiments, uptake of the target protein (e.g., receptor) into the cell is increased by at least 400%, relative to uptake of the target protein (e.g., receptor) when the agent that binds the target protein is not conjugated to the promoter peptide. In certain embodiments, uptake of the target protein (e.g., receptor) into the cell is increased by at least 600%, relative to uptake of the target protein (e.g., receptor) when the agent that binds the target protein is not conjugated to the promoter peptide. In certain embodiments, uptake of the target protein (e.g., receptor) into the cell is increased by at least 800%, relative to uptake of the target protein (e.g., receptor) when the agent that binds the target protein is not conjugated to the promoter peptide. In certain embodiments, uptake of the target protein (e.g., receptor) into the cell is increased by at least 1000%, relative to uptake of the target protein (e.g., receptor) when the agent that binds the target protein is not conjugated to the promoter peptide.

In certain embodiments, the promoter peptide promotes uptake of the target protein (e.g., receptor) into the cell, whereas the target protein is not internalized into the cell in a statistically significant amount when contacted with an agent that binds the target protein not conjugated to the promoter peptide.

In certain embodiments, the target protein is CD38; and the antibody is an antibody directed against CD38, or an antigen-binding fragment thereof. In certain embodiments, the target protein is CD38; and the antibody is a monoclonal antibody (mAb) directed against CD38, or an antigen-binding fragment thereof. In certain embodiments, the target protein is CD38; and the antibody is daratumumab, or an antigen-binding fragment thereof. In certain embodiments, the target protein is CD38; and the antibody is daratumumab. Other non-limiting examples of anti-CD38 antibodies are provided herein.

In certain embodiments, the target protein is CD20; and the antibody is an antibody directed against CD20, or an antigen-binding fragment thereof. In certain embodiments, the target protein is CD20; and the antibody is a monoclonal antibody (mAb) directed against CD20, or an antigen-binding fragment thereof. In certain embodiments, the target protein is CD20; and the antibody is obinutuzumab, or an antigen-binding fragment thereof. In certain embodiments, the target protein is CD20; and the antibody is obinutuzumab. Other non-limiting examples of anti-CD20 antibodies are provided herein.

In certain embodiments, the receptor is HER2; and the antibody is an antibody directed against HER2, or an antigen-binding fragment thereof. In certain embodiments, the receptor is HER2; and the antibody is a monoclonal antibody (mAb) directed against HER2, or an antigen-binding fragment thereof. In certain embodiments, the receptor is HER2; and the antibody is trastuzumab, or an antigen-binding fragment thereof. In certain embodiments, the receptor is HER2; and the antibody is trastuzumab. Other non-limiting examples of anti-HER2 antibodies are provided herein.

In certain embodiments, the receptor is HER3; and the antibody is an antibody directed against HER3, or an antigen-binding fragment thereof. In certain embodiments, the receptor is HER3; and the antibody is a monoclonal antibody (mAb) directed against HER3, or an antigen-binding fragment thereof. In certain embodiments, the receptor is HER3; and the antibody is patritumab, or an antigen-binding fragment thereof. In certain embodiments, the receptor is HER3; and the antibody is patritumab.

In certain embodiments, the receptor is Folate Receptor Alpha; and the antibody is an antibody directed against Folate Receptor Alpha, or an antigen-binding fragment thereof. In certain embodiments, the receptor is Folate Receptor Alpha; and the antibody is a monoclonal antibody (mAb) directed against Folate Receptor Alpha, or an antigen-binding fragment thereof. In certain embodiments, the receptor is Folate Receptor Alpha; and the antibody is mirvetuximab, or an antigen-binding fragment thereof. In certain embodiments, the receptor is Folate Receptor Alpha; and the antibody is mirvetuximab.

In certain embodiments, the receptor is EGFR; and the antibody is an antibody directed against EGFR, or an antigen-binding fragment thereof. In certain embodiments, the receptor is EGFR; and the antibody is a monoclonal antibody (mAb) directed against EGFR, or an antigen-binding fragment thereof. In certain embodiments, the receptor is EGFR; and the antibody is cetuximab, or an antigen-binding fragment thereof. In certain embodiments, the receptor is EGFR; and the antibody is cetuximab. Other non-limiting examples of anti-EGFR antibodies are provided herein.

Promoter peptides are described herein. In certain embodiments, the promoter peptide is a stapled peptide (e.g., a singly stapled, doubly stapled, or stitched peptide). In certain embodiments, the promoter peptide is a stapled Magainin peptide. In certain embodiments, the promoter peptide is a stapled Magainin II peptide. In certain embodiments, the promoter peptide is a stapled peptide comprising SEQ ID NO: 1 (wherein X¹ and X² are connected via a crosslink, and X³ and X⁴ are connected via a crosslink). In certain embodiments, the promoter peptide is a stapled peptide comprising SEQ ID NO: 2 (wherein X¹ and X² are connected via a crosslink, and X³ and X⁴ are connected via a crosslink).

In certain embodiments, the target protein is CD38; the antibody is a monoclonal antibody (mAb) directed against CD38, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the target protein is CD38; the antibody is daratumumab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the target protein is CD20; the antibody is a monoclonal antibody (mAb) directed against CD20, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the target protein is CD20; the antibody is obinutuzumab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the receptor is HER2; the antibody is a monoclonal antibody (mAb) directed against HER2, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink). In certain embodiments, the receptor is HER2; the antibody is trastuzumab, or an antigen-binding fragment thereof; and the promoter peptide is a stapled peptide comprising SEQ ID NO: 1 or SEQ ID NO: 2 (wherein X¹ and X² are connected via a crosslink and X³ and X⁴ are connected via a crosslink).

In certain embodiments, the receptor is EGFR; the antibody is a monoclonal antibody (mAb) directed against EGFR, or an antigen-binding fragment thereof; and the promoter peptide is a stapled peptide comprising SEQ ID NO: 1 or SEQ ID NO: 2 (wherein X¹ and X² are connected via a crosslink, and X³ and X⁴ are connected via a crosslink). In certain embodiments, the receptor is EGFR; the antibody is cetuximab, or an antigen-binding fragment thereof; and the promoter peptide is a stapled peptide comprising SEQ ID NO: 1 or SEQ ID NO: 2 (wherein X¹ and X² are connected via a crosslink, and X³ and X⁴ are connected via a crosslink).

Additional embodiments and non-limiting examples of peptide-antibody conjugates (PACs) for receptor internalization are provided herein, including in Table 4, Table 2A, and Table 2B. Further non-limiting examples of PACs for receptor internalization are provided in Table 2C, Table 3C, and Table 3D.

TABLE 4
Examples of PACs for Target Protein Internalization

| | Promoter | | | Promoter | | | |
|---|---|---|---|---|---|---|---|
| | Peptide | | | Peptide | | | |
| | SEQ ID | X¹-X² | X³-X⁴ | C- | Linker | | Target |
| PAC# | NO. | crosslink | Crosslink | Terminus | Number | Antibody | Protein |
| 1 | 2 | alk | alk | —NH₂ | L5 | daratumumab | CD38 |
| 2 | 2 | alk | alk | —NH₂ | L5 | obinutuzumab | CD20 |
| 3 | 2 | alk | alk | —NH₂ | L5 | trastuzumab | HER2 |
| 4 | 2 | alk | alk | —NH₂ | L5 | cetuximab | EGFR |
| 174 | 2 | alk | alk | —NH₂ | L5 | Nimotuzumab | EGFR |
| 175 | 2 | alk | alk | —NH₂ | L5 | Panitumumab | EGFR |
| 176 | 142 | alk | N/A | —NH₂ | L5 | cetuximab | EGFR |
| 177 | 154 | alk | alk | —NH₂ | L5 | cetuximab | EGFR |
| 178 | 160 | alk | N/A | —NH₂ | L5 | cetuximab | EGFR |
| 179 | 165 | alk | N/A | —NH₂ | L5 | cetuximab | EGFR |

In certain embodiments, the target protein (e.g., receptor) is degraded after entry into the cell. The conjugates provided herein can therefore be used to degrade target proteins (e.g., receptors), leading to a decreased level of the target protein on or in a cell and/or in a subject. As used herein, “degrade,” “degradation,” and the like, in the context of a protein refers to metabolizing or breaking down said protein. In some embodiments, degrading a protein (e.g., a target protein) leads to a decreased level of the intact and/or active protein in a subject or cell. In some embodiments, degrading a protein (e.g., a target protein) leads to decreased activity of the protein or a downstream effect, e.g., relative to a baseline or control level of activity. As used herein, “level” means the amount or concentration of a protein compared to a reference. The reference can be any useful reference, as defined herein. “Decreased level” of a protein means a decrease in amount or concentration of a protein, as compared to a reference. In certain embodiments, a decreased level is a decrease by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or about 100%. In certain embodiments, a decreased level is a decrease by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, or 100%. A level of a protein may be expressed in mass/vol (e.g., g/dL, mg/mL, g/mL, ng/mL) or percentage relative to total protein in a sample or cell.

### Conjugates

The present disclosure provides conjugates, including peptide-antibody conjugates (PACs) described herein. In general, conjugates (e.g., PACs) provided herein comprise a promoter peptide (e.g., stapled or unstapled promoter peptide) conjugated to an agent (e.g., an antibody or antigen-binding fragment thereof). In certain embodiments, the promoter peptide is conjugated to the agent (e.g., antibody or antigen-binging fragment thereof) via a linker (e.g., a cleavable linker or non-cleavable linker).

### Antibodies and Antigen-Binding Fragments

The peptide-antibody conjugates (PACs) provided herein comprise an antibody or antigen-binding fragment thereof. As used herein, the term “antibody” refers to a molecule that specifically binds to, or is immunologically reactive with, a particular antigen and includes at least the variable domain of a heavy chain, and normally includes at least the variable domains of a heavy chain and a light chain of an immunoglobulin. Unless otherwise indicated, the term “antibody” (Ab) is meant to include both intact (whole) molecules as well as antibody fragments (e.g., Fab and F(ab′)₂ fragments) that are capable of specifically binding to a target antigen. Antibodies (including intact antibodies and antigen-binding fragments), variants, and derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments (e.g., Fab, Fab′, and F(ab′)₂, Fd, Fvs, single-chain Fvs (scFv), rlgG, single-chain antibodies, disulfide-linked Fvs (sdFv), fragments containing either a V_{L} or V_{H} domain, fragments produced by an Fab expression library), and anti-idiotypic (anti-Id) antibodies. Fab and F(ab′)₂ fragments, for example, lack the Fc fragment of an intact antibody. Antibody molecules of the conjugates can be of any type (e.g., IgG, IgE, IgM, IgD, IgA, and IgY), class (e.g., IgG1, IgG2, IgG3, IgG4, IgA1 and IgA2) or subclass of immunoglobulin molecule.

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be, e.g., a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb (Ward et al., Nature, 1989, 341, 544-546) including V_{H} and V_{L} domains; (vi) a dAb fragment that consists of a V_{H} domain; (vii) a dAb that consists of a V_{H} or a V_{L} domain; (viii) an isolated complementarity determining region (CDR); and (ix) a combination of two or more isolated CDRs which may optionally be joined by a linker, e.g., a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv)). These antibody fragments (i.e., “antigen-binding fragments”) can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

Antibodies described herein can be murine, rat, human, or of any other origin (including chimeric or humanized antibodies and fragments thereof). Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In certain embodiments, the antibody is a monoclonal antibody (mAb) or antigen-binding fragment thereof. In certain embodiments the antibody is an intact (i.e., whole) mAb. In certain embodiments, the antibody is an antigen-binding fragment of a mAb. Examples of monoclonal antibodies (mAbs) (including generic name, trade name(s), known target antigen(s), and exemplary use(s)) are provided below in Table 5. Therapeutic applications of the mAbs listed below are not limited the particular known target antigens and exemplary uses provided.

TABLE 5
Examples of Monoclonal Antibodies (mAbs)

| | | Known Target | |
|---|---|---|---|
| Generic Name | Trade Name(s) | Antigen(s) | Exemplary Use(s) |
| 3F8 | | GD2 ganglioside | neuroblastoma |
| Abagovomab | | CA-125 | ovarian cancer |
| Abciximab | ReoPro | CD41 (integrin alpha-IIb) | platelet aggregation inhibitor |
| Abituzumab | | CD51 | cancer |
| Abrezekimab | | interleukin 13 | |
| Abrilumab | | integrin α₄ β₇ | inflammatory bowel |
| | | | disease, ulcerative colitis, Crohn's |
| | | | disease |
| Actoxumab | | Clostridium difficile | Clostridium difficile colitis |
| Adalimumab | Humira | TNF-α | rheumatoid arthritis, Crohn's disease, |
| | | | plaque psoriasis, psoriatic |
| | | | arthritis, ankylosing |
| | | | spondylitis, juvenile idiopathic |
| | | | arthritis, hemolytic disease of the |
| | | | newborn |
| Adecatumumab | | EpCAM | prostate and breast cancer |
| Aducanumab | Aduhelm | beta-amyloid | Alzheimer's disease |
| Afasevikumab | | IL17A and IL17F | multiple sclerosis |
| Afelimomab | | TNF-α | sepsis |
| Alacizumab | | VEGFR2 | cancer |
| Alemtuzumab | Lemtrada, Campath | CD52 | multiple sclerosis |
| Alirocumab | Praluent | PCSK9 | hypercholesterolemia |
| Altumomab | Hybri-ceaker | Carcinoembryonic | colorectal cancer |
| | (Altumomab | antigen (CEA) | |
| | pentetate) | | |
| Amatuximab | | mesothelin | cancer |
| Amivantamab | Rybrevant | Epidermal growth factor | non-small cell lung cancer (NSCLC) |
| | | receptor (EGFR), cMet | |
| Anatumomab | | Tumor-associated | non-small cell lung cancer |
| | | glycoprotein 72 (TAG-72) | |
| Andecaliximab | | gelatinase B | gastric cancer, gastroesophageal |
| | | | cancer |
| Anetumab | | mesothelin (MSLN) | cancer |
| Anifrolumab | Saphnelo | interferon α/β receptor | systemic lupus erythematosus |
| Ansuvimab | Ebanga | Ebola virus glycoprotein | Ebola virus |
| Anrukinzumab | | IL-13 | asthma |
| Apolizumab | | HLA-DR | hematological cancers |
| Aprutumab | | FGFR2 | |
| Arcitumomab | CEA-Scan | Carcinoembryonic | gastrointestinal cancers |
| | | antigen (CEA) | |
| Ascrinvacumab | | activin receptor-like | cancer |
| | | kinase 1 | |
| Aselizumab | | L-selectin (CD62L) | severely injured patients |
| Atezolizumab | Tecentriq | PD-L 1 | bladder, non-small cell lung, and |
| | | | triple-negative breast cancers |
| Atidortoxumab | | Staphylococcus | |
| | | aureus alpha toxin | |
| Atinumab | | RTN4 | |
| Atoltivimab | | | Zaire ebolavirus (Ebola virus) |
| Atorolimumab | | Rhesus factor | hemolytic disease of the newborn |
| Avelumab | Bavencio | PD-L1 | urothelial carcinoma and Merkel cell |
| | | | carcinoma |
| Azintuxizumab | | CD319 | cancer |
| Bamlanivimab | | spike protein receptor | coronavirus disease 2019 (COVID- |
| | | binding domain (RBD) of | 19) |
| | | SARS-CoV-2 | |
| Bapineuzumab | | beta amyloid | Alzheimer's disease |
| Basiliximab | Simulect | CD25 (α chain of IL-2 | prevention of organ transplant |
| | | receptor) | rejections |
| Bavituximab | | phosphatidylserine | cancer, viral infections |
| BCD-100 | | PD-1 | melanoma |
| Bectumomab | LymphoScan | CD22 | non-Hodgkin's lymphoma (detection) |
| Begelomab | | DPP4 | |
| Belantamab | Blenrep | B-cell maturation | multiple myeloma |
| | (Belantamab | antigen (BCMA) | |
| | mafodotin) | | |
| Belimumab | Benlysta | B-cell activating | systemic lupus erythematosus without |
| | | factor (BAFF) | renal or CNS involvement |
| Bemarituzumab | | FGFR2 | gastric cancer or gastroesophageal |
| | | | junction adenocarcinoma |
| Benralizumab | Fasenra | CD125 | asthma |
| Berlimatoxumab | | Staphylococcus aureus bi- | |
| | | component leukocidin | |
| Bermekimab | Xilonix | IL1A | colorectal cancer |
| Bersanlimab | | ICAM-1 | |
| Bertilimumab | | CCL11 (eotaxin-1) | severe allergic disorders |
| Besilesomab | Scintimun | Carcinoembryonic | inflammatory lesions and metastases |
| | | antigen (CEA)-related | (detection) |
| | | antigen | |
| Bevacizumab | Avastin | VEGF | colorectal, non-small cell lung, renal, |
| | | | glioblastoma, and ovarian cancers |
| Bezlotoxumab | Zinplava | Clostridium difficile | Clostridium difficile colitis |
| Biciromab | FibriScint | fibrin II, beta chain | thromboembolism (diagnosis) |
| Bimagrumab | | ACVR2B | myostatin inhibitor |
| Bimekizumab | Bimzelx | IL17A, IL17F, IL17AF | psoriasis |
| Birtamimab | | serum amyloid A protein | amyloidosis |
| Bivatuzumab | | CD44 v6 | squamous cell carcinoma |
| Bleselumab | | CD40 | organ transplant rejection |
| Blinatumomab | Blincyto | CD19 | pre-B Acute lymphoblastic |
| | | | leukemia (ALL) (CD19+) |
| Blontuvetmab | Blontress | CD20 | |
| Blosozumab | | SOST | osteoporosis |
| Bococizumab | | PCSK9 | dyslipidemia |
| Brazikumab | | IL23 | Crohn's disease |
| Brentuximab | Adcentris | CD30 (TNFRSF8) | Hodgkin's lymphoma, |
| | (Brentuximab | | anaplastic large-cell lymphoma |
| | vedotin) | | |
| Briakinumab | | IL-12, IL-23 | psoriasis, rheumatoid |
| | | | arthritis, inflammatory bowel |
| | | | diseases, multiple sclerosis |
| Brodalumab | Siliq | IL-17 | plaque psoriasis |
| Brolucizumab | Beovu | vascular endothelial | wet age-related macular degeneration |
| | | growth factor A (VEGFA) | |
| Brontictuzumab | | Notch 1 | cancer |
| Burosumab | Crysvita | FGF 23 | X-linked hypophosphatemia |
| Cabiralizumab | | CSF1R | metastatic pancreatic cancer |
| Camidanlumab | | CD25 (α chain of IL-2 | B-cell Hodgkin's lymphoma, non- |
| | | receptor) | Hodgkin lymphoma, acute |
| | | | lymphoblastic leukemia, acute |
| | | | myeloid leukemia |
| Camrelizumab | | PD-1 | hepatocellular carcinoma |
| Canakinumab | Ilaris | IL-1 | cryopyrin-associated periodic |
| | | | syndrome |
| Cantuzumab | | CanAg (a glycoform | cancer |
| | | of MUC1) | |
| Caplacizumab | Cablivi | VWF | thrombotic thrombocytopeni |
| | | | purpura, thrombosis |
| Casirivimab | | spike protein receptor | coronavirus disease 2019 (COVID- |
| | | binding domain (RBD) of | 19) |
| | | SARS-CoV-2 | |
| Capromab | Prostascint | Glutamate | prostate cancer |
| | | carboxypeptidase II | |
| Carlumab | | MCP-1 | oncology/immune indications |
| Carotuximab | | endoglin | angiosarcoma |
| Catumaxomab | Removab | EpCAM, CD3 | ovarian cancer, malignant ascites, |
| | | | gastric cancer |
| cBR96 | | Lewis-Y antigen | cancer |
| Cedelizumab | | CD4 | prevention of organ transplant |
| | | | rejections, treatment of autoimmune |
| | | | diseases |
| Cemiplimab | Libtayo | PD-1 | cutaneous squamous cell carcinoma |
| Cergutuzumab | | IL2 | cancer |
| Certolizumab | Cimzia | TNF-α | Crohn's disease, rheumatoid |
| | (Certolizumab | | arthritis, axial |
| | pegol) | | spondyloarthritis, psoriasis arthritis |
| Cetrelimab | | PD-1 | cancer |
| Cetuximab | Erbitux | Epidermal growth factor | colorectal cancer and head and neck |
| | | receptor (EGFR) | squamous cell carcinoma |
| Cibisatamab | | CEACAM5 | cancer |
| Cirmtuzumab | | ROR1 | chronic lymphocytic leukemia |
| Citatuzumab | | EpCAM | ovarian cancer and other solid tumors |
| Cixutumumab | | IGF-1 receptor (CD221) | solid tumors |
| Clazakizumab | | Interleukin 6 (IL-6) | rheumatoid arthritis |
| Clenoliximab | | CD4 | rheumatoid arthritis |
| Clivatuzumab | hPAM4-Cide | MUC1 | pancreatic cancer |
| | (Clivatuzumab | | |
| | tetraxetan) | | |
| Codrituzumab | | glypican 3 | cancer |
| Cofetuzumab | | PTK7 | cancer |
| Coltuximab | | CD19 | cancer |
| Conatumumab | | TRAIL-R2 | cancer |
| Concizumab | | tissue factor pathway | bleeding |
| | | inhibitor (TFPI) | |
| Cosfroviximab | ZMapp | ebolavirus glycoprotein | Ebola virus |
| Crenezumab | | 1-40-β-amyloid | Alzheimer's disease |
| Crizanlizumab | Adakveo | P-selectin | sickle-cell disease |
| Crotedumab | | glucagon | diabetes |
| | | receptor (GCGR) | |
| CR6261 | | Influenza A | infectious disease/influenza A |
| | | hemagglutinin | |
| Cusatuzumab | | CD70 | cancer |
| Dacetuzumab | | CD40 | hematologic cancers |
| Daclizumab | Zenapax | CD25 (α chain of IL-2 | prevention of organ transplant |
| | | receptor) | rejections, multiple sclerosis |
| Dalotuzumab | | IGF-1 receptor (CD221) | cancer |
| Dapirolizumab | | CD154 (CD40L) | |
| Daratumumab | Darzalex | CD38 | multiple myeloma |
| Dectrekumab | | IL-13 | |
| Demcizumab | | DLL4 | cancer |
| Denintuzumab | | CD19 | cancer |
| Denosumab | Prolia | RANKL | osteoporosis, bone metastases |
| Depatuxizumab | | EGFR | glioblastoma |
| Derlotuximab | | histone complex | recurrent glioblastoma multiforme |
| Detumomab | | B-lymphoma cell | lymphoma |
| Dezamizumab | | serum amyloid P | |
| | | component | |
| Dinutuximab | Unituxin | GD2 ganglioside | neuroblastoma |
| Dinutuximab | Qarziba | GD2 ganglioside | neuroblastoma |
| beta | | | |
| Diridavumab | | hemagglutinin | influenza A |
| Domagrozumab | | GDF-8 | Duchenne muscular dystrophy |
| Dorlimomab | | | |
| Dostarlimab | Jemperli | PD-1 | endometrial cancer |
| Drozitumab | | DR5 | cancer |
| DS-8201 | | HER2 | gastric or gastroesophageal |
| | | | junction adenocarcinoma |
| Duligotuzumab | | ERBB3 (HER3) | testicular cancer |
| Dupilumab | Dupixent | IL-4Rα | atopic dermatitis, asthma, nasal |
| | | | polyps |
| Durvalumab | Imfinzi | PD-L1 | bladder cancer |
| Dusigitumab | | IL GF2 | B-cell malignancies |
| Duvortuxizumab | | CD19, CD3E | cancer |
| Ecromeximab | | GD3 ganglioside | malignant melanoma |
| Eculizumab | Soliris | C5 | paroxysmal nocturnal |
| | | | hemoglobinuria, atypical hemolytic |
| | | | uremic syndrome |
| Edobacomab | | endotoxin | sepsis caused by Gram- |
| | | | negative bacteria |
| Edrecolomab | Panorex | EpCAM | colorectal carcinoma |
| Efalizumab | Raptiva | LFA-1 (CD11a) | psoriasis |
| Efungumab | Mycograb | Hsp90 | invasive Candida infection |
| Eldelumab | | interferon gamma-induced | Crohn's disease, ulcerative colitis |
| | | protein | |
| Elezanumab | | repulsive guidance | spinal cord injury and multiple |
| | | molecule A (RGMA) | sclerosis |
| Elgemtumab | | ERBB3 (HER3) | cancer |
| Elotuzumab | Empliciti | SLAMF7 | multiple myeloma |
| Elsilimomab | | IL-6 | |
| Emactuzumab | | CSF1R | cancer |
| Emapalumab | Gamifant | interferon gamma | hemophagocytic lymphohistiocytosis |
| Emibetuzumab | | hHGFR | cancer |
| Emicizumab | Hemlibra | activated F9, F10 | hemophilia A |
| Enapotamab | | AXL | cancer |
| Enavatuzumab | | TWEAK receptor | cancer |
| Enfortumab | Padcev | nectin-4 | bladder cancer |
| | (Enfortumab | | |
| | vedotin) | | |
| Enlimomab | | ICAM-1 (CD54) | |
| Enoblituzumab | | CD276 | cancer |
| Enokizumab | | IL9 | asthma |
| Enoticumab | | DLL4 | |
| Ensituximab | | MUC5AC | cancer |
| Epcoritamab | | CD3, CD20 | B-cell lymphoma |
| Epitumomab | | episialin | |
| Epratuzumab | | CD22 | cancer, systemic lupus |
| | | | erythematosus (SLE) |
| Eptinezumab | Vyepti | calcitonin gene-related | migraine |
| | | peptide | |
| Erenumab | Aimovig | calcitonin gene-related | migraine |
| | | peptide receptor (CGRP) | |
| Erlizumab | | ITGB2 (CD18) | heart attack, stroke, traumatic shock |
| Ertumaxomab | Rexomun | HER2/neu, CD3 | breast cancer |
| Etaracizumab | Abegrin | integrin αᵥβ₃ | melanoma, prostate cancer, ovarian |
| | | | cancer etc. |
| Etesevimab | | spike protein receptor | coronavirus disease 2019 (COVID- |
| | | binding domain (RBD) of | 19) |
| | | SARS-CoV-2 | |
| Etigilimab | | TIGIT | |
| Etrolizumab | | integrin β₇ | inflammatory bowel disease |
| Evinacumab | Evkeeza | angiopoietin 3 | dyslipidemia |
| Evolocumab | Repatha | PCSK9 | hypercholesterolemia |
| Exbivirumab | | hepatitis B surface antigen | hepatitis B |
| Fanolesomab | Neutro Spec | CD15 | appendicitis |
| Faralimomab | | interferon receptor | |
| Faricimab | | VEGF-A and Ang-2 | angiogenesis, ocular vascular diseases |
| Farletuzumab | | folate receptor 1 | ovarian cancer |
| Fasinumab | | Nerve growth | acute sciatic pain |
| | | factor (HNGF) | |
| FBTA05 | Lymphomun | CD20 | chronic lymphocytic leukemia |
| Felvizumab | | respiratory syncytial virus | respiratory syncytial virus infection |
| Fezakinumab | | IL-22 | rheumatoid arthritis, psoriasis |
| Fibatuzumab | | ephrin receptor A3 | |
| Ficlatuzumab | | Hepatocyte growth | cancer |
| | | factor (HGF) | |
| Figitumumab | | IGF-1 receptor (CD221) | adrenocortical carcinoma, non-small |
| | | | cell lung carcinoma etc. |
| Firivumab | | influenza A virus | |
| | | hemagglutinin | |
| Flanvotumab | | TYRP1 (glycoprotein 75) | melanoma |
| Fletikumab | | IL 20 | rheumatoid arthritis |
| Flotetuzumab | | IL 3 receptor | hematological malignancies |
| Fontolizumab | HuZAF | IFN-γ | Crohn's disease |
| Foralumab | | CD3 epsilon | |
| Foravirumab | | rabies virus glycoprotein | rabies (prophylaxis) |
| Fremanezumab | Ajovy | calcitonin gene-related | migraine |
| | | peptide alpha and beta | |
| Fresolimumab | | TGF-β | idiopathic pulmonary fibrosis, focal |
| | | | segmental glomerulosclerosis, cancer |
| Frovocimab | | PCSK9 | hypercholesterolemia |
| Frunevetmab | | nerve growth | |
| | | factor (NGF) | |
| Fulranumab | | Nerve growth | pain |
| | | factor (NGF) | |
| Futuximab | | Epidermal growth factor | cancer |
| | | receptor (EGFR) | |
| Galcanezumab | Emgality | calcitonin | migraine |
| Galiximab | | CD80 | B-cell lymphoma |
| Gancotamab | | HER2/neu | cancer |
| Ganitumab | | IGF-1 receptor (CD221) | cancer |
| Gantenerumab | | beta amyloid | Alzheimer's disease |
| Gatipotuzumab | | MUC1 | cancer |
| Gavilimomab | | CD147 (basigin) | graft versus host disease |
| Gedivumab | | hemagglutinin HA | |
| Gemtuzumab | Mylotarg | CD33 | acute myelogenous leukemia |
| | (Gemtuzumab | | |
| | ozogamicin) | | |
| Gevokizumab | | IL-1ß | diabetes |
| Gilvetmab | | PCDC1 | |
| Gimsilumab | | CSF2 | rheumatoid arthritis |
| Girentuximab | Rencarex | carbonic anhydrase | clear cell renal cell carcinoma |
| | | 9 (CA-IX) | |
| Glembatumumab | | GPNMB | melanoma, breast cancer |
| Golimumab | Simponi | TNF-α | rheumatoid arthritis, psoriatic |
| | | | arthritis, ankylosing spondylitis |
| Gomiliximab | | CD23 (IgE receptor) | allergic asthma |
| Gosuranemab | | tau protein | progressive supranuclear palsy |
| Guselkumab | Tremfya | IL 23 | psoriasis |
| Ianalumab | | BAFF-R | autoimmune hepatitis |
| Ibalizumab | Trogarzo | CD4 | HIV infection |
| Sintilimab | | PD-1 | squamous cell non-small cell lung |
| | | | cancer |
| Ibritumomab | Zevalin | CD20 | non-Hodgkin's lymphoma |
| | (Ibritumomab | | |
| | tiuxetan) | | |
| Icrucumab | | VEGFR-1 | cancer etc. |
| Idarucizumab | Praxbind | dabigatran | reversal of anticoagulant effects of |
| | | | dabigatran |
| Ifabotuzumab | | EPHA3 | glioblastoma multiforme |
| Igovomab | Indimacis-125 | CA-125 | ovarian cancer |
| Iladatuzumab | | CD79B | cancer |
| Imalumab | | macrophage migration | cancer |
| | | inhibitory factor (MIF) | |
| Imaprelimab | | melanoma cell adhesion | |
| | | molecule (MCAM) | |
| Imciromab | Myoscint | cardiac myosin | cardiac imaging |
| Imdevimab | | spike protein receptor | coronavirus disease 2019 (COVID- |
| | | binding domain (RBD) of | 19) |
| | | SARS-CoV-2 | |
| Imgatuzumab | | Epidermal growth factor | cancer |
| | | receptor (EGFR) | |
| Inclacumab | | selectin P | cardiovascular disease |
| Indatuximab | | SDC1 | cancer |
| Indusatumab | | GUCY2C | cancer |
| Inebilizumab | Uplizna | CD19 | cancer, systemic sclerosis, multiple |
| | | | sclerosis |
| Infliximab | Remicade | TNF-α | rheumatoid arthritis, ankylosing |
| | | | spondylitis, psoriatic arthritis, |
| | | | psoriasis, Crohn's disease, ulcerative |
| | | | colitis |
| Intetumumab | | CD51 | solid tumors (prostate cancer, |
| | | | melanoma) |
| Inolimomab | | CD25 (α chain of IL-2 | graft versus host disease |
| | | receptor) | |
| Inotuzumab | Besponsa | CD22 | acute lymphoblastic leukemia (ALL) |
| | (Inotuzumab | | |
| | ozogamicin) | | |
| Ipilimumab | Yervoy | CD152, CTLA-4 | melanoma and renal cell carcinoma |
| Iomab-B | | CD45 | ablation of bone marrow |
| Iratumumab | | CD30 (TNFRSF8) | Hodgkin's lymphoma |
| Isatuximab | Sarclisa | CD38 | multiple myeloma |
| Iscalimab | | CD40 | |
| Istiratumab | | IGF1R, CD221 | advanced solid tumors |
| Itolizumab | Alzumab | CD6 | psoriasis |
| Ixekizumab | Taltz | IL 17A | autoimmune diseases |
| Keliximab | | CD4 | chronic asthma |
| Labetuzumab | CEA-Cide | Carcinoembryonic | colorectal cancer |
| | | antigen (CEA) | |
| Lacnotuzumab | | CSF1, macrophage colony | cancer |
| | | stimulating factor (MCSF) | |
| Ladiratuzumab | | LIV-1 | cancer |
| Lampalizumab | | Complement factor | geographic atrophy secondary to |
| | | D (CFD) | agerelated macular degeneration |
| Lanadelumab | Takhzyro | kallikrein | angioedema |
| Landogrozumab | | GDF-8 | muscle wasting disorders |
| Laprituximab | | epidermal growth factor | |
| | | receptor (EGFR) | |
| Larcaviximab | | ebolavirus glycoprotein | Ebola virus |
| Lebrikizumab | | IL-13 | asthma |
| Lemalesomab | | NCA-90 (granulocyte | diagnostic agent |
| | | antigen) | |
| Lendalizumab | | C5 | |
| Lenvervimab | | hepatitis B surfage | hepatitis B |
| | | antigen | |
| Lenzilumab | | CSF2 | chronic myelomonocytic |
| | | | leukemia and juvenile |
| | | | myelomonocytic leukemia |
| Lerdelimumab | | TGF beta 2 | reduction of scarring after glaucoma |
| | | | surgery |
| Leronlimab | | CCR5 | breast cancer, HIV |
| Lesofavumab | | hemagglutinin HA | |
| Letolizumab | | tumor necrosis factor | inflammatory diseases |
| | | related activation | |
| | | protein (TRAP) | |
| Lexatumumab | | TRAIL-R2 | cancer |
| Libivirumab | | hepatitis B surface antigen | hepatitis B |
| Lifastuzumab | | phosphate-sodium co- | cancer |
| | | transporter (NaPi2B) | |
| Ligelizumab | | IGHE | severe asthma and chronic |
| | | | spontaneous urticaria |
| Loncastuximab | Zynlonta | CD19 | diffuse large B-cell lymphoma |
| | (Loncastuximab | | |
| | tesirine) | | |
| Losatuxizumab | | epidermal growth receptor | cancer |
| | | factor (EGRF), ERBB1 H | |
| | | ER1 | |
| Lilotomab | | CD37 | cancer |
| Lintuzumab | | CD33 | cancer |
| Lirilumab | | KIR2D | solid and hematological cancers |
| Lodelcizumab | | PCSK9 | hypercholesterolemia |
| Lokivetmab | Cytopoint | Canis lupus | clinical signs of atopic dermatitis in |
| | | familiaris IL31 | dogs |
| Lorvotuzumab | | CD56 | cancer |
| Lucatumumab | | CD40 | multiple myeloma, non-Hodgkin's |
| | | | lymphoma, Hodgkin's lymphoma |
| Lulizumab | | CD28 | autoimmune diseases |
| Lumiliximab | | CD23 (IgE receptor) | chronic lymphocytic leukemia |
| Lumretuzumab | | ERBB3 (HER3) | cancer |
| Lupartumab | | | |
| Lupartumab | | LYPD3 | |
| Lutikizumab | | interleukin 1 alpha | |
| Maftivimab | | | Zaire ebolavirus (Ebola virus) |
| Mapatumumab | | TRAIL-R1 | cancer |
| Margetuximab | Margenza | HER2 | breast cancer |
| Marstacimab | | tissue factor pathway | bleeding with hemophilia |
| | | inhibitor (TFPI) | |
| Maslimomab | | T-cell receptor | |
| Mavrilimumab | | GMCSF receptor a-chain | rheumatoid arthritis |
| Matuzumab | | Epidermal growth factor | colorectal, lung and stomach cancer |
| | | receptor (EGFR) | |
| Mepolizumab | Bosatria | IL-5 | asthma and white blood cell diseases |
| Metelimumab | | TGF beta 1 | systemic scleroderma |
| Milatuzumab | | CD74 | multiple myeloma and other |
| | | | hematological malignancies |
| Minretumomab | | TAG-72 | tumor detection and therapy |
| Mirikizumab | | IL23A | psoriasis |
| Mirvetuximab | | folate receptor alpha | ovarian cancer |
| soravtansine | | | |
| Mitumomab | | GD3 ganglioside | small cell lung carcinoma |
| Modotuximab | | EGFR extracellular | cancer |
| | | domain III | |
| Mogamulizumab | Poteligeo | CCR4 | cutaneous T-cell lymphoma |
| Monalizumab | | NKG2A | rheumatoid arthritis, gynecologic |
| | | | malignancies, and other cancers |
| Morolimumab | | Rhesus factor | |
| Mosunetuzumab | | CD3E, MS4A1, CD20 | cancer |
| Motavizumab | Numax | respiratory syncytial virus | respiratory syncytial virus |
| | | | (prevention) |
| Moxetumomab | Lumoxiti | CD22 | hairy cell leukemia |
| | (Moxetumomab | | |
| | pasudotox) | | |
| Muromonab- | Orthoclone OKT3 | CD3 | prevention of organ transplant |
| CD3 | | | rejections |
| Nacolomab | | C242 antigen | colorectal cancer |
| Namilumab | | CSF2 | |
| Naptumomab | | 5T4 | non-small cell lung carcinoma, renal |
| | | | cell carcinoma |
| Naratuximab | | CD37 | |
| Narnatumab | | MSTIR (aka RON) | cancer |
| Natalizumab | Tysabri | integrin α₄ | multiple sclerosis, Crohn's disease |
| Navicixizumab | | DLL4 and VEGFA | cancer |
| Navivumab | | influenza A | |
| | | virus hemagglutinin HA | |
| Naxitamab | Danyelza | c-Met, GD2 | neuroblastoma |
| Nebacumab | | endotoxin | sepsis |
| Necitumumab | Portrazza | Epidermal growth factor | Non-small cell lung cancer |
| | | receptor (EGFR) | |
| Nemolizumab | | IL31RA | eczema |
| NEOD001 | | amyloid | primary systemic amyloidosis |
| Nerelimomab | | TNF-α | |
| Nesvacumab | | angiopoietin 2 | cancer |
| Netakimab | Efleira | interleukin 17A | plaque psoriasis |
| Nimotuzumab | BioMab-EGFR, | epidermal growth factor | squamous cell carcinoma, head and |
| | Theracim, Theraloc | receptor (EGFR) | neck cancer, nasopharyngeal |
| | | | cancer, glioma |
| Nirsevimab | | RSV fusion glycoprotein | respiratory syncytial virus |
| Nivolumab | Opdivo | PD-1 | melanoma, lung, and renal cancers |
| Nofetumomab | Verluma | | cancer |
| | (Nofetumomab | | |
| | merpentan) | | |
| Obiltoxaximab | Anthim | Bacillus anthracis anthrax | Bacillus anthracis spores |
| Generic Name | Trade Name(s) | Known Target | Exemplary Use(s) |
| | | Antigen(s) | |
| Obinutuzumab | Gazyva | CD20 | chronic lymphocytic leukemia |
| Ocaratuzumab | | CD20 | cancer |
| Ocrelizumab | Ocrevus | CD20 | multiple sclerosis |
| Odesivimab | | | Zaire ebolavirus (Ebola virus) |
| Odulimomab | | LFA-1 (CD11a) | prevention of organ transplant |
| | | | rejections, immunological diseases |
| Ofatumumab | Arzerra, Kesimpta | CD20 | chronic lymphocytic leukemia, |
| | | | multiple sclerosis |
| Olaratumab | Lartruvo | PDGFRα | Sarcoma |
| Oleclumab | | 5′-nucleotidase | pancreatic and colorectal cancer |
| Olendalizumab | | complement C5a | systemic lupus erythematosus, lupus |
| | | | nephritis, acute graft-versus-hose |
| | | | disease |
| Olokizumab | | IL6 | rheumatoid arthritis |
| Omalizumab | Xolair | IgE Fc region | allergic asthma |
| Omburtamab | | CD276 | cancer |
| OMS721 | | MASP-2 | atypical hemolytic uremic syndrome |
| Onartuzumab | | human scatter factor | cancer |
| | | receptor kinase | |
| Ontuxizumab | | TEM1 | cancer |
| Onvatilimab | | VISTA (protein) (VSIR) | |
| Opicinumab | | LINGO-1 | multiple sclerosis |
| Oportuzumab | Vicinium | EpCAM | bladder cancer |
| | (Oportuzumab | | |
| | monatox) | | |
| Oregovomab | OvaRex | CA-125 | ovarian cancer |
| Orticumab | | oxLDL | |
| Otelixizumab | | CD3 | diabetes mellitus type 1 |
| Otilimab | | GMCSF | osteoarthritis, rheumatoid arthritis |
| Otlertuzumab | | CD37 | cancer |
| Oxelumab | | OX-40 | asthma |
| Ozanezumab | | NOGO-A | ALS and multiple sclerosis |
| Ozoralizumab | | TNF-α | inflammation |
| Pagibaximab | | lipoteichoic acid | sepsis (Staphylococcus) |
| Palivizumab | Synagis, | F protein of respiratory | respiratory syncytial virus |
| | Abbosynagis | syncytial virus | (prevention) |
| Pamrevlumab | | connective tissue growth | idiopathic pulmonary fibrosis (IPF), |
| | | factor (CTGF) | pancreatic cancer |
| Panitumumab | Vectibix | epidermal growth factor | colorectal cancer |
| | | receptor (EGFR) | |
| Pankomab | | tumor specific | ovarian cancer |
| | | glycosylation of MUC1 | |
| Panobacumab | | Pseudomonas aeruginosa | Pseudomonas aeruginosa infection |
| Parsatuzumab | | EGFL7 | cancer |
| Pascolizumab | | IL-4 | asthma |
| Pasotuxizumab | | folate hydrolase | cancer |
| Pateclizumab | | lymphotoxin alpha (LTA) | TNF |
| Patritumab | | ERBB3 (HER3) | cancer |
| PDR001 | | PD-1 | melanoma |
| Pembrolizumab | Keytruda | PD-1 | melanoma and other cancers |
| Pemtumomab | Theragyn | MUC1 | cancer |
| Penpulimab | | PD-1 | cancer |
| Perakizumab | | IL 17A | arthritis |
| Pertuzumab | Perjeta | HER2 | breast cancer |
| Pexelizumab | | C5 | reduction of side effects of cardiac |
| | | | surgery |
| Pidilizumab | | PD-1 | cancer and infectious diseases |
| Pinatuzumab | | CD22 | cancer |
| Pintumomab | | adenocarcinoma antigen | adenocarcinoma |
| Placulumab | | human TNF | pain and inflammatory diseases |
| Prezalumab | | human TNF | |
| Plozalizumab | | CCR2 | diabetic |
| | | | nephropathy and arteriovenous graft |
| | | | patency |
| Pogalizumab | | tumor necrosis factor | |
| | | receptor (TNFR) | |
| | | superfamily member 4 | |
| Polatuzumab | Polivy | CD79B | B-cell lymphoma |
| | (Polatuzumab | | |
| | vedotin) | | |
| Ponezumab | | human beta-amyloid | Alzheimer's disease |
| Porgaviximab | | Zaire ebolavirus | Ebola virus disease |
| | | glycoprotein | |
| Prasinezumab | | Alpha-synuclein | Parkinson's disease |
| Prezalizumab | | inducible T-cell co- | |
| | | stimulatory | |
| | | ligand (ICOSL) | |
| Priliximab | | CD4 | Crohn's disease, multiple sclerosis |
| Pritoxaximab | | E. coli shiga toxin type-1 | |
| Pritumumab | | vimentin | brain cancer |
| PRO 140 | | CCR5 | HIV infection |
| Quilizumab | | IGHE | asthma |
| Racotumomab | Vaxira | NGNA ganglioside | non-small cell lung cancer |
| Radretumab | | fibronectin extra domain- | cancer |
| | | B | |
| Rafivirumab | | rabies virus glycoprotein | rabies (prophylaxis) |
| Ralpancizumab | | PCSK9 | dy slipidemia |
| Ramucirumab | Cyramza | VEGFR2 | gastric cancer |
| Ranevetmab | | NGF | osteoarthritis in dogs |
| Ranibizumab | Lucentis | VEGF-A | macular degeneration (wet form) |
| Raxibacumab | | anthrax toxin, protective | anthrax (prophylaxis and treatment) |
| | | antigen | |
| Ravagalimab | | CD40 | Crohn's disease |
| Ravulizumab | Ultomiris | C5 | paroxysmal nocturnal |
| | | | hemoglobinuria, atypical hemolytic |
| | | | uremic syndrome |
| Refanezumab | | myelin-associated | recovery of motor function after |
| | | glycoprotein | stroke |
| Regavirumab | | cytomegalovirus glycopro | cytomegalovirus infection |
| | | tein B | |
| Regdanvimab | Regkirona | spike protein receptor | coronavirus disease 2019 (COVID- |
| | | binding domain (RBD) of | 19) |
| | | SARS-CoV-2 | |
| Relatlimab | | LAG3 | melanoma |
| Remtolumab | | interleukin 17 alpha, TNF | |
| Reslizumab | Cinqair | IL-5 | inflammations of the airways, skin |
| | | | and gastrointestinal tract |
| Rilotumumab | | hepatocyte growth | solid tumors |
| | | factor (HGF) | |
| Rinucumab | | platelet-derived growth | neovascular age-related macular |
| | | factor receptor beta | degeneration |
| Risankizumab | Skyrizi | IL23A | Crohn's disease, psoriasis, psoriatic |
| | | | arthritis, and asthma |
| Rituximab | MabThera, Rituxan | CD20 | B-Cell Lymphoma |
| Rivabazumab | | Pseudomonas | |
| | | aeruginosa type III | |
| | | secretion system | |
| Robatumumab | | IGF-1 receptor (CD221) | cancer |
| Rmab | RabiShield | rabies virus G | post-exposure prophylaxis of rabies |
| | | glycoprotein | |
| Roledumab | | RHD (gene) (RHD) | Rh disease |
| Romilkimab | | interleukin 13 | |
| Romosozumab | Evenity | sclerostin | osteoporosis |
| Rontalizumab | | IFN-α | systemic lupus erythematosus |
| Rosmantuzumab | | root plate-specific | cancer |
| | | spondin 3 | |
| Rovalpituzumab | | DLL3 | small cell lung cancer |
| Rovelizumab | LeukArrest | CD11, CD18 | haemorrhagic shock etc. |
| Rozanolixizumab | | FCGRT | Immune thrombocytopenia |
| | | | purpura (ITP), myasthenia gravis |
| Ruplizumab | Antova | CD154 (CD40L) | rheumatic diseases |
| SA237 | | IL-6R | neuromyelitis optica and |
| | | | neuromyelitis optica spectrum |
| | | | disorders |
| Sacituzumab | Trodelvy | TROP-2 | triple-negative breast cancer |
| | (Sacituzumab | | |
| | govitecan) | | |
| Samalizumab | | CD200 | cancer |
| Samrotamab | | LRRC15 | cancer |
| Sarilumab | Kevzara | IL6 | rheumatoid arthritis, ankylosing |
| | | | spondylitis |
| Satralizumab | Enspryng | IL6 receptor | neuromyelitis optica |
| Satumomab | | TAG-72 | cancer |
| Secukinumab | Cosentyx | IL 17A | uveitis, rheumatoid arthritis psoriasis |
| Selicrelumab | | CD40 | |
| Seribantumab | | ERBB3 (HER3) | cancer |
| Setoxaximab | | E. coli shiga toxin type-2 | |
| Setrusumab | | sclerostin (SOST) | |
| Sevirumab | | cytomegalovirus | cytomegalovirus infection |
| Sibrotuzumab | | FAP (gene) (FAP) | cancer |
| SGN-CD19A | | CD19 | acute lymphoblastic leukemia and B- |
| | | | cell non-Hodgkin lymphoma |
| SHP647 | | mucosal addressin cell | Crohn's disease |
| | | adhesion molecule | |
| Sifalimumab | | IFN-α | systemic lupus |
| | | | erythematosus (SLE), |
| | | | dermatomyositis, polymyositis |
| Siltuximab | Sylvant | IL-6 | cancer |
| Simtuzumab | | LOXL2 | fibrosis |
| Siplizumab | | CD2 | psoriasis, graft-versus-host |
| | | | disease (prevention) |
| Sirtratumab | | SLITRK6 | cancer |
| Sirukumab | | IL-6 | rheumatoid arthritis |
| Sofituzumab | | CA-125 | ovarian cancer |
| Solanezumab | | beta amyloid | Alzheimer's disease |
| Solitomab | | EpCAM | gastrointestinal, lung, and other |
| | | | cancers |
| Sonepcizumab | | sphingosine-1-phosphate | choroidal and retinal |
| | | | neovascularization |
| Sontuzumab | | episialin | |
| Sotrovimab | Xevudy | spike protein receptor | coronavirus disease 2019 (COVID- |
| | | binding domain (RBD) of | 19) |
| | | SARS-CoV-2 | |
| Spartalizumab | | PDCD1, CD279 | melanoma |
| Stamulumab | | myostatin | muscular dystrophy |
| Sulesomab | LeukoScan | NCA-90 (granulocyte | osteomyelitis |
| | | antigen) | |
| Suptavumab | | RSVFR | medically attended lower respiratory |
| | | | disease |
| Sutimlimab | | C1s | cold agglutinin disease |
| Suvizumab | | HIV-1 | viral infections |
| Suvratoxumab | | Staphylococcus | nosocomial pneumonia |
| | | aureus alpha toxin | |
| Tabalumab | | B-cell activating | B-cell cancers |
| | | factor (BAFF) | |
| Tacatuzumab | AFP-Cide | alpha-fetoprotein | cancer |
| | (Tacatuzumab | | |
| | tetraxetan) | | |
| Tadocizumab | | integrin α_{IIb}β₃ | percutaneous coronary intervention |
| Tafasitamab | Monjuvi | CD19 | Diffuse large B-cell lymphoma |
| Talacotuzumab | | CD123 | leukemia |
| Talizumab | | IgE | allergic reaction |
| Talquetamab | | GPRC5D, CD3 | relapsed or refractory multiple |
| | | | myeloma |
| Tamtuvetmab | Tactress | CD52 | |
| Tanezumab | | nerve growth | pain |
| | | factor (NGF) | |
| Taplitumomab | | CD19 | cancer |
| Tarextumab | | Notch receptor | cancer |
| Tavolimab | | CD134 | cancer |
| Teclistamab | | B-cell maturation | relapsed or refractory multiple |
| | | antigen (BCMA), CD3 | myeloma |
| Tefibazumab | Aurexis | clumping factor A | Staphylococcus aureus infection |
| Telimomab | | | |
| aritox | | | |
| Telisotuzumab | | HGFR | cancer |
| Telisotuzumab | | HGFR | cancer |
| Tenatumomab | | tenascin C | cancer |
| Teneliximab | | CD40 | autoimmune diseases and prevention |
| | | | of organ transplant rejection |
| Teplizumab | | CD3 | diabetes mellitus type 1 |
| Tepoditamab | | dendritic cell- | cancer |
| | | associated lectin 2 | |
| Teprotumumab | Tepezza | IGF-1 receptor (CD221) | thyroid eye disease |
| Tesidolumab | | C5 | |
| Tetulomab | | CD37 | cancer |
| Tezepelumab | | thymic stromal | asthma, atopic dermatitis |
| | | lymphopoietin (TSLP) | |
| TGN1412 | | CD28 | chronic lymphocytic |
| | | | leukemia, rheumatoid arthritis |
| Tibulizumab | | B-cell activating | autoimmune disorders |
| | | factor (BAFF) | |
| Tildrakizumab | Ilumya | IL23 | immunologically mediated |
| | | | inflammatory disorders |
| Tigatuzumab | | TRAIL-R2 | cancer |
| Timigutuzumab | | HER2 | cancer |
| Timolumab | | AOC3 | |
| tiragolumab | | | |
| Tiragotumab | | TIGIT | cancer |
| Tislelizumab | | PCDC1, CD279 | non-small cell lung cancer |
| Tisotumab | Tivdak (Tisotumab | coagulation factor III | Cervical cancer |
| | vedotin) | | |
| TNX-650 | | IL-13 | Hodgkin's lymphoma |
| Tocilizumab | Actemra, | IL-6 receptor | rheumatoid arthritis |
| | RoActemra | | |
| Tomuzotuximab | | epidermal growth factor | cancer |
| | | receptor (EGFR), HER1 | |
| Toralizumab | | CD154 (CD40L) | rheumatoid arthritis, lupus nephritis, |
| | | | etc. |
| Toripalimab | Tuoyi | PD-1 | cancer |
| Tosatoxumab | | Staphylococcus aureus | |
| Tositumomab | Bexxar | CD20 | Non-Hodgkin's lymphoma |
| Tovetumab | | PDGFRA | cancer |
| Tralokinumab | Adtralza | IL-13 | atopic dermatitis |
| Trastuzumab | Herceptin, Kadcyla | HER2 | breast cancer |
| | (Trastuzumab | | |
| | duocarmazine), | | |
| | Kadcyla | | |
| | (Trastuzumab | | |
| | emtansine) | | |
| TRBS07 | Ektomab | GD2 ganglioside | melanoma |
| Tregalizumab | | CD4 | |
| Tremelimumab | | CTLA-4 | non-small cell lung, head & neck, |
| | | | urothelial cancer |
| Trevogrumab | | growth differentiation | muscle atrophy due to orthopedic |
| | | factor 8 | disuse and sarcopenia |
| Tucotuzumab | | EpCAM | cancer |
| Tuvirumab | | hepatitis B virus | chronic hepatitis B |
| Ublituximab | | MS4A1 | multiple sclerosis, chronic |
| | | | lymphocytic leukemia |
| Ulocuplumab | | CXCR4 (CD184) | hematologic malignancies |
| Urelumab | | 4-1BB (CD137) | cancer |
| Urtoxazumab | | Escherichia coli | diarrhoea caused by E. coli |
| Ustekinumab | Stelara | IL-12, IL-23 | multiple sclerosis, psoriasis, psoriatic |
| | | | arthritis |
| Utomilumab | | 4-1BB (CD137) | diffuse large B-cell lymphoma |
| Vadastuximab | | CD33 | acute myeloid leukemia |
| Vanalimab | | CD40 | |
| Vandortuzumab | | STEAP1 | cancer |
| Vantictumab | | Frizzled receptor | cancer |
| Vanucizumab | | angiopoietin 2 | cancer |
| Vapaliximab | | AOC3 (VAP-1) | |
| Varisacumab | | VEGF-A | angiogenesis |
| Varlilumab | | CD27 | solid tumors and hematologic |
| | | | malignancies |
| Vatelizumab | | ITGA2 (CD49b) | |
| Vedolizumab | Entyvio | integrin α₄ β₇ | Crohn's disease, ulcerative colitis |
| Veltuzumab | | CD20 | non-Hodgkin's lymphoma |
| Vepalimomab | | AOC3 (VAP-1) | inflammation |
| Vesencumab | | NRP1 | solid malignancies |
| Visilizumab | Nuvion | CD3 | Crohn's disease, ulcerative colitis |
| Vobarilizumab | | IL6R | inflammatory autoimmune diseases |
| Volociximab | | integrin α₅β₁ | solid tumors |
| Vonlerolizumab | | CD134 | cancer |
| Vopratelimab | | CD278, aka ICOS | |
| Vorsetuzumab | | CD70 | cancer |
| Votumumab | HumaSPECT | tumor | colorectal tumors |
| | | antigen CTAA16.88 | |
| Vunakizumab | | interleukin 17 alpha | |
| Xentuzumab | | IGF1, IGF2 | |
| XMAB-5574 | | CD19 | diffuse large B-cell lymphoma |
| Zalutumumab | | Epidermal growth factor | squamous cell carcinoma of the head |
| | | receptor (EGFR) | and neck |
| Zanolimumab | | CD4 | rheumatoid arthritis, psoriasis, T-cell |
| | | | lymphoma |
| Zatuximab | | HER1 | cancer |
| Zenocutuzumab | | ERBB3, HER3 | cancer |
| Ziralimumab | | CD147 (basigin) | |
| Zolbetuximab | | Claudin 18 Isoform 2 | gastric cancer, gastrointestinal |
| | | | adenocarcinoma, and pancreatic |
| | | | cancer |
| Zolimomab | | CD5 | systemic lupus erythematosus, graft- |
| | | | versus-host disease |
| humanized 11B6 | | KLK-2 | prostate cancer |
| rosopatamab | | PSMA | cancer |

In certain embodiments, the antibody is an anti-cancer antibody or antigen-binding fragment thereof. As used herein, “anti-cancer antibody” refers to an antibody that targets an antigen expressed on a cancer cell (e.g., a cancer-associated antigen or cancer-specific antigen). In certain embodiments, the antibody is an anti-cancer mAb or an antigen-binding fragment thereof. Table 6 below provides examples of anti-cancer mAbs (including generic name, target antigen(s), and exemplary use(s)).

Other non-limiting examples of anti-cancer mAbs are provided above in Table 5. Therapeutic applications of the anti-cancer mAbs listed below are not limited to the particular known target antigen(s) and exemplary use(s) provided.

TABLE 6
Examples of Anti-Cancer Monoclonal Antibodies (mAbs)

| Generic Name | Target Antigen(s) | Exemplary Use(s) |
|---|---|---|
| Atezolizumab | PD-L1 | bladder, non-small cell lung, and triple-negative breast cancers |
| Avelumab | PD-L1 | urothelial carcinoma and merkel cell carcinoma |
| Bevacizumab | VEGF | colorectal, non-small cell lung, renal, glioblastoma, and ovarian |
| | | cancers |
| Cemiplimab | PD-1 | cutaneous squamous cell carcinoma |
| Cetuximab | EGFR | colorectal cancer and head and neck squamous cell carcinoma |
| Daratumumab | CD38 | multiple myeloma |
| Dinutuximab | GD2 | neuroblastoma |
| Durvalumab | PD-L1 | bladder cancer |
| Elotuzumab | SLAMF7 | multiple myeloma |
| Ipilimumab | CTLA-4 | melanoma and renal cell carcinoma |
| Isatuximab | CD38 | multiple myeloma |
| Mogamulizumab | CCR4 | cutaneous T-cell lymphoma |
| Necitumumab | EGFR | non-small cell lung cancer |
| Nivolumab | PD-1 | melanoma, lung, and renal cancers |
| Obinutuzumab | CD20 | chronic lymphocytic leukemia |
| Ofatumumab | CD20 | chronic lymphocytic leukemia |
| Olaratumab | PDGFRα | sarcoma |
| Panitumumab | EGFR | colorectal cancer |
| Pembrolizumab | PD-1 | melanoma and other cancers |
| Pertuzumab | HER2 | breast cancer |
| Ramucirumab | VEGFR2 | gastric cancer |
| Rituximab | CD20 | B-cell lymphoma |
| Trastuzumab | HER2 | breast cancer |
| Gemtuzumab | CD33 | acute myeloid leukemia |
| Brentuximab | CD30 | Hodgkin's lymphoma and anaplastic large-cell lymphoma |
| Inotuzumab | CD22 | acute lymphoblastic leukemia |
| Polatuzumab | CD79B | B-cell lymphoma |
| Enfortumab | Nectin-4 | bladder cancer |
| Sacituzumab | TROP2 | triple negative breast cancer |
| Moxetumomab | CD22 | hairy-cell leukemia |
| Ibritumomab | CD20 | non-Hodgkin's lymphoma |
| tositumomab | CD20 | non-Hodgkin's lymphoma |
| Blinatumomab | CD19, CD3 | acute lymphoblastic leukemia |
| Tisotumab | Tissue factor | cervical cancer |
| Amivantamab | EGFR, cMET | non-small cell lung cancer |
| Loncastuximab | CD19 | diffuse large B-cell lymphoma |
| Dostarlimab | PD-1 | endometrial cancer |
| Margetuximab | HER2 | breast cancer |
| Naxitamab | GD2 | neuroblastoma |
| Belantamab | BCMA | multiple myeloma |
| Tafasitamab | CD19 | diffuse large B-cell lymphoma |

In certain embodiments, the antibody is Belantamab, CH1A1, Elgemtumab, Gemtuzumab, Inebilizumab, L6, Labetuzumab, Lifastuzumab, Lintuzumab, Pembrolizumab, Pertuzumab, Polatuzumab, Rituximab, Seribantumab, Tafasitamab, or Tusamitamab, or an antigen-binding fragment thereof.

In certain embodiments, the antibody is a homolog of an antibody provided herein, or an antigen-binding fragment thereof. As used herein, the term “homolog” refers to an antibody of similar amino acid composition or sequence to the disclosed antibody, allowing for variations that do not have an adverse effect on the ability of the antibody to carry out its normal function (e.g., binding to a target antigen). Homologs may be the same length, shorter, or longer than the disclosed antibody. Homologs may have at least about 60% (e.g., at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or at least about 99%) sequence identity (i.e., homology) to the amino acid sequence of the disclosed antibody. A homolog can be, for example, an antibody sequence that is modified by deletion, addition, mutation, or substitution of one or more amino acid residues.

In certain embodiments, the antibody is a homolog of trastuzumab, or an antigen-binding fragment thereof. In certain embodiments, the antibody is a homolog of daratumumab, or an antigen-binding fragment thereof. In certain embodiments, the antibody is a homolog of obinutuzumab, or an antigen-binding fragment thereof. In certain embodiments, the antibody is a homolog of obinutuzumab, or an antigen-binding fragment thereof. In certain embodiments, the antibody is a homolog of cetuximab, or an antigen-binding fragment thereof.

In certain embodiments, the antibody or antigen-binding fragment thereof comprises an engineered cysteine residue (e.g., for conjugation of the promoter peptide to the cysteine). Such antibodies are sometimes referred to as THIOMAB™ antibodies. For example, in some embodiments, the antibody is Vadastuximab comprising an S239C mutation.

Antibodies not disclosed herein and/or not yet known in the art may be used in the PACs provided herein.

In certain embodiments, the antibody is an antibody-drug conjugate (ADC) or antigen-binding fragment thereof. In such embodiments, the PAC comprises an antibody or antigen-binding fragment thereof conjugated to (i) the promoter peptide; and (ii) an additional pharmaceutically active agent (i.e., the “drug” component of the ADC). For example, a PAC provided herein can comprise a promoter peptide conjugated to trastuzumab emtansine (i.e., wherein trastuzumab is conjugated to (i) the promoter peptide; and (ii) emtansine). Table 3 above provides examples of antibody-drug conjugates (ADCs) (including generic name, trade name, and exemplary use(s)). Any of these ADCs may be conjugated to a promoter peptide to form a PAC provided herein.

As used herein, “antigen” refers to an entity (e.g., protein) which is targeted by and binds an antibody. An “antigen” described herein may be a “target protein” described herein, and vice versa. In some instances, antigens trigger the immune system to produce antibodies against the antigens in what is known as an immune response. In certain embodiments, an antigen (e.g., target protein) is on the surface of a cell (e.g., a cancer cell). In certain embodiments, an antigen (e.g., target protein) described herein is a cluster of differentiation (CD) protein (e.g., CD2, CD3, CD4, CD5, CD6, CD8, CD11, CD11a (LFA-1), CD15, CD18 (ITGB2), CD19, CD20 (MS4A1), CD22, CD23, CD25, CD27, CD28, CD30, CD33, CD37, CD38, CD40, CD41, CD44, CD49b (ITGA2), CD51, CD52, CD54 (ICAM-1), CD56, CD62L, CD70, CD74, CD79B, CD80, CD125, CD140a, CD142, CD147, CD152 (CTLA4), CD154, CD200, CD221, CD240D, CD248, CD257 (BAFF), CD274 (PD-L1), CD276, CD279 (PD-1)). Other examples of antigens (e.g., target proteins) include, but are not limited to, glycoproteins (e.g., TROP2, TPBG, EpCAM, CEA, gpA33, Mucins, TAG-72, CA-IX, CA-125 (MUC16), PSMA, endoglin, fibronectin, MUC1, mucin CanAg, rabies virus glycoprotein), glycolipids (e.g., gangliosides (e.g., GD2, GD3, GM2), myelin-associated glycoprotein, TAG-72, TN-C, TYRP1), carbohydrates (e.g., Lewis-Y²), folate binding proteins (e.g., folate receptor 1, folate receptor alpha), vascular targets (e.g., VEGF, VEGFR, αVβ3, α5β1, VAP-1, VEGF-A, VEGFR-1, VEGFR-2), growth factors (e.g., HGF, IGF-1, NGF, HNGF, TGF-β, TGF-β1, TGF-β2, EGFL7, GDF-8), growth factor receptors (e.g., EGFR/ERBB1/HER1, ERBB2/HER2, ERBB3/HER3, HGFR/c-Met, HGFR, HHGFR, IGF-1 receptor, PDGF-R^{aa}, PDGF-RP, EphA3, TRAIL-R1, TRAIL-R2 (DR5), RANKL), stromal and extracellular matrix antigens (e.g., FAP, Tensacin), activin receptor (e.g., ACVR2B), activin receptor-like kinase (e.g., activin receptor-like kinase 2), angiopoietin (e.g., angiopoietin-2, angiopoietin-3), interferons (e.g., INF-α, INF-β, INF-γ), interleukins (e.g., IL 17A, IL 17F, IL20, IL-12, IL-23, IL-13, IL-17, IL-10, IL-22, IL-4, IL-5, IL-6, IL-6 receptor, IL-2, IL-23A, IL-31RA, IL-4, IL-6, IL-9, ILGF2), integrins (e.g., α₄β₇, α₅β₁, α₇β₇, α_{IIb}β₃, αᵥβ₃), complement component (e.g., C5, CFD), chemokines (e.g., CCL11, CCL2 (MCP-1)), chemokine receptors (e.g., CCR2, CCR4, CCR5), Notch receptors (e.g., Notch 1, NRP1), virulence factor (e.g., ClfA), colony stimulating factor (e.g., CSF2), colony stimulating factor receptors (e.g., CSF1R), delta-like ligands (e.g., DLL3, DLL4), Lipopolysaccharides (e.g., endotoxins), human leukocyte antigen (e.g., HLA-DR), heat shock proteins (e.g., Hsp90), SLAM proteins (e.g., SLAMF7), tissue factor pathway inhibitors, tumor necrosis factors (e.g., TNF-α), tumor necrosis factor receptors (TNFR superfamily member 4), microphage migration inhibitory factor, rhesus factor, neurite outgrowth inhibitor, alpha-fetoprotein, amyloid beta, carcinoembryonic antigen (CEA), neural apoptosis-regulated proteinase 1, Ch4D5, CLDN18.2, LOXL2, MSLN, NCA-90, PCSK9, sclerostin, syndecan 1, STEAP1, TSLP, TWEAK receptor, tumor antigen CTAA16.88, nectin-4, and TM4SF1. Other examples of known antigens (e.g., target protein) can be found in, e.g., Table 5 and Table 6 above.

In certain embodiments, the antigen is an antigen (e.g., target protein) expressed on a cancer cell. Different classes of antigens (e.g., target proteins) expressed on cancer cells include: (i) “cancer-associated antigens” (CAAs), meaning antigens expressed on cancer cells that can also be present on normal cells; (ii) “cancer-specific antigens” (CSAs), meaning antigens expressed on cancer cells that are not found on normal cells; (iii) “tumor-associated antigens” (TAAs), meaning antigens expressed on solid tumor cells that can also be present on normal cells; and (iv) “tumor-specific antigens” (TSAs), meaning antigens expressed on solid tumor cells that are not found on normal cells.

### Promoter Peptides

Conjugates (e.g., PACs) for internalization into a cell as provided herein comprise promoter peptides. In certain embodiments, the promoter peptide is a stapled peptide. In certain embodiments, the promoter peptide is an unstapled peptide.

A promoter peptide (e.g., stapled or unstapled promoter peptide) can be of any length. In certain embodiments, the promoter peptide (e.g., stapled or unstapled promoter peptide) is 100 amino acids or fewer in length. In certain embodiments, the promoter peptide (e.g., stapled or unstapled promoter peptide) is 90 amino acids or fewer in length. In certain embodiments, the promoter peptide (e.g., stapled or unstapled promoter peptide) is 80 amino acids or fewer in length. In certain embodiments, the promoter peptide (e.g., stapled or unstapled promoter peptide) is 70 amino acids or fewer in length. In certain embodiments, the promoter peptide (e.g., stapled or unstapled promoter peptide) is 60 amino acids or fewer in length. In certain embodiments, the promoter peptide (e.g., stapled or unstapled promoter peptide) is 50 amino acids or fewer in length. In certain embodiments, the promoter peptide (e.g., stapled or unstapled promoter peptide) is 45 amino acids or fewer in length. In certain embodiments, the promoter peptide (e.g., stapled or unstapled promoter peptide) is 40 amino acids or fewer in length. In certain embodiments, the promoter peptide (e.g., stapled or unstapled promoter peptide) is 35 amino acids or fewer in length. In certain embodiments, the promoter peptide (e.g., stapled or unstapled promoter peptide) is 30 amino acids or fewer in length. In certain embodiments, the promoter peptide (e.g., stapled or unstapled promoter peptide) is 25 amino acids or fewer in length. In certain embodiments, the promoter peptide (e.g., stapled or unstapled promoter peptide) is 20 amino acids or fewer in length. In certain embodiments, the promoter peptide (e.g., stapled or unstapled promoter peptide) is 19, 20, 21, 22, 23, 24, 25, 26, 27, 28, 29, 30, 31, 32, 33, 34, 35, 36, 37, 38, 39, or 40 amino acids or fewer in length. In certain embodiments, the stapled promoter peptide (e.g., stapled or unstapled promoter peptide) is at least the length of an amino acid sequence provided herein. In certain embodiments, the stapled peptide is the length of any amino acid sequence provided herein.

In certain embodiments, the promoter peptide (e.g., stapled or unstapled promoter peptide) is 5 to 40 amino acids, inclusive, in length. In certain embodiments, the promoter peptide (e.g., stapled or unstapled promoter peptide) is 5 to 30 amino acids, inclusive, in length. In certain embodiments, the promoter peptide (e.g., stapled or unstapled promoter peptide) is 10 to 30 amino acids, inclusive, in length. In certain embodiments, the promoter peptide (e.g., stapled or unstapled promoter peptide) is 5 to 20 amino acids, inclusive, in length. In certain embodiments, the promoter peptide (e.g., stapled or unstapled promoter peptide) is 7 to 20 amino acids, inclusive, in length. In certain embodiments, the promoter peptide (e.g., stapled or unstapled promoter peptide) is 10 to 20 amino acids, inclusive, in length. In certain embodiments, the promoter peptide (e.g., stapled or unstapled promoter peptide) is 12 to 16 amino acids, inclusive, in length. In certain embodiments, the promoter peptide (e.g., stapled or unstapled promoter peptide) is 15 to 30 amino acids, inclusive, in length. In certain embodiments, the promoter peptide (e.g., stapled or unstapled promoter peptide) is 20 to 30 amino acids, inclusive, in length. In certain embodiments, the promoter peptide (e.g., stapled or unstapled promoter peptide) is 22 to 28 amino acids, inclusive, in length. In certain embodiments, the promoter peptide (e.g., stapled or unstapled promoter peptide) is 10, 11, 12, 13, 14, 15, 16, 17, 18, 19, 20, 21, 22, 23, 24, 25, 26, 27, 28, 29, or 30 amino acids in length.

### Cationic Promoter Peptides

In certain embodiments, the promoter peptide (e.g., stapled or unstapled promoter peptide) comprises a minimum number of charged amino acids (referred to herein as “cationic promoter peptides”). “Charged amino acids,” as used herein, refers to amino acids with nitrogen-containing sidechains which are capable of being protonated (and thus charged) at a certain pH, or which are permanently charged (e.g., trimethyl lysine). Non-limiting examples of charged amino acids include arginine, histidine, lysine, Dap, Dab, Orn, homo-lysine, homo-arginine, homo-histidine, 4-guanidino-phenylalanine, and 4-amino-phenylalanine. “Non-charged amino acid” refers to any amino acid that does not include a nitrogen-containing sidechain.

In certain embodiments, the promoter peptide (e.g., stapled or unstapled promoter peptide) comprises at least 3 charged amino acids. In certain embodiments, the promoter peptide (e.g., stapled or unstapled promoter peptide) comprises at least 4 charged amino acids. In certain embodiments, the promoter peptide (e.g., stapled or unstapled promoter peptide) comprises at least 5 charged amino acids. In certain embodiments, the promoter peptide (e.g., stapled or unstapled promoter peptide) comprises at least 6 charged amino acids. In certain embodiments, the promoter peptide (e.g., stapled or unstapled promoter peptide) comprises at least 7 charged amino acids. In certain embodiments, the promoter peptide (e.g., stapled or unstapled promoter peptide) comprises at least 8 charged amino acids. In certain embodiments, the promoter peptide (e.g., stapled or unstapled promoter peptide) comprises at least 9 charged amino acids. In certain embodiments, the promoter peptide (e.g., stapled or unstapled promoter peptide) comprises at least 10 charged amino acids.

In certain embodiments, the promoter peptide (e.g., stapled or unstapled promoter peptide) comprises at least 3 amino acids independently selected from lysine, Dab, Dap, and Orn. In certain embodiments, the promoter peptide (e.g., stapled or unstapled promoter peptide) comprises at least 4 amino acids independently selected from lysine, Dab, Dap, and Orn. In certain embodiments, the promoter peptide (e.g., stapled or unstapled promoter peptide) comprises at least 5 amino acids independently selected from lysine, Dab, Dap, and Orn. In certain embodiments, the promoter peptide (e.g., stapled or unstapled promoter peptide) comprises at least 6 amino acids independently selected from lysine, Dab, Dap, and Orn. In certain embodiments, the promoter peptide (e.g., stapled or unstapled promoter peptide) comprises at least 7 amino acids independently selected from lysine, Dab, Dap, and Orn. In certain embodiments, the promoter peptide (e.g., stapled or unstapled promoter peptide) comprises at least 8 amino acids independently selected from lysine, Dab, Dap, and Orn. In certain embodiments, the promoter peptide (e.g., stapled or unstapled promoter peptide) comprises at least 9 amino acids independently selected from lysine, Dab, Dap, and Orn. In certain embodiments, the promoter peptide (e.g., stapled or unstapled promoter peptide) comprises at least 10 amino acids independently selected from lysine, Dab, Dap, and Orn.

In certain embodiments, the promoter peptide (e.g., stapled or unstapled promoter peptide) comprises at least 3 Dap. In certain embodiments, the promoter peptide (e.g., stapled or unstapled promoter peptide) comprises at least 4 Dap. In certain embodiments, the promoter peptide (e.g., stapled or unstapled promoter peptide) comprises at least 5 Dap. In certain embodiments, the promoter peptide (e.g., stapled or unstapled promoter peptide) comprises at least 6 Dap. In certain embodiments, the promoter peptide (e.g., stapled or unstapled promoter peptide) comprises at least 7 Dap. In certain embodiments, the promoter peptide (e.g., stapled or unstapled promoter peptide) comprises at least 7 Dap. In certain embodiments, the promoter peptide (e.g., stapled or unstapled promoter peptide) comprises at least 8 Dap. In certain embodiments, the promoter peptide (e.g., stapled or unstapled promoter peptide) comprises at least 9 Dap. In certain embodiments, the promoter peptide (e.g., stapled or unstapled promoter peptide) comprises at least 10 Dap.

In certain embodiments, the promoter peptide (e.g., stapled or unstapled promoter peptide) comprises a ratio of charged amino acids to non-charged amino acids of 1:4 to 1:1, inclusive. In certain embodiments, the promoter peptide (e.g., stapled or unstapled promoter peptide) comprises a ratio of charged amino acids to non-charged amino acids of 1:3 to 1:1, inclusive. In certain embodiments, the promoter peptide (e.g., stapled or unstapled promoter peptide) comprises a ratio of charged amino acids to non-charged amino acids of 1:2 to 1:1, inclusive. In certain embodiments, the charged amino acids are independently selected from lysine, Dab, Dap, and Orn. In certain embodiments, the charged amino acids are Dap.

In certain embodiments, the promoter peptide (e.g., stapled or unstapled promoter peptide) comprises a ratio of charged amino acids to non-charged amino acids of 1:4 to 1:1.5, inclusive. In certain embodiments, the promoter peptide (e.g., stapled or unstapled promoter peptide) comprises a ratio of charged amino acids to non-charged amino acids of 1:3 to 1:1.5, inclusive. In certain embodiments, the promoter peptide (e.g., stapled or unstapled promoter peptide) comprises a ratio of charged amino acids to non-charged amino acids of 1:2 to 1:1.5, inclusive. In certain embodiments, the promoter peptide (e.g., stapled or unstapled promoter peptide) comprises a ratio of charged amino acids to non-charged amino acids of 1:1.8 to 1:1.5, inclusive. In certain embodiments, the promoter peptide (e.g., stapled or unstapled promoter peptide) comprises a ratio of charged amino acids to non-charged amino acids of 1:1.7 to 1:1.6, inclusive. In certain embodiments, the charged amino acids are independently selected from lysine, Dab, Dap, and Orn. In certain embodiments, the charged amino acids are Dap.

In certain embodiments, the promoter peptide (e.g., stapled or unstapled promoter peptide) comprises a ratio of charged amino acids to non-charged amino acids of 1:1.9 to 1:1, inclusive. In certain embodiments, the promoter peptide (e.g., stapled or unstapled promoter peptide) comprises a ratio of charged amino acids to non-charged amino acids of 1:1.8 to 1:1, inclusive. In certain embodiments, the promoter peptide (e.g., stapled or unstapled promoter peptide) comprises a ratio of charged amino acids to non-charged amino acids of 1:1.7 to 1:1, inclusive. In certain embodiments, the promoter peptide (e.g., stapled or unstapled promoter peptide) comprises a ratio of charged amino acids to non-charged amino acids of 1:1.6 to 1:1, inclusive. In certain embodiments, the promoter peptide (e.g., stapled or unstapled promoter peptide) comprises a ratio of charged amino acids to non-charged amino acids of 1:1.5 to 1:1, inclusive. In certain embodiments, the promoter peptide (e.g., stapled or unstapled promoter peptide) comprises a ratio of charged amino acids to non-charged amino acids of 1:1.4 to 1:1.1, inclusive. In certain embodiments, the promoter peptide (e.g., stapled or unstapled promoter peptide) comprises a ratio of charged amino acids to non-charged amino acids of 1:1.4 to 1:1.2, inclusive. In certain embodiments, the charged amino acids are independently selected from lysine, Dab, Dap, and Orn. In certain embodiments, the charged amino acids are Dap.

In certain embodiments, the promoter peptide comprises the amino acid sequence:

(SEQ ID NO: 5)
X⁵X⁶X⁷X⁵X⁶X⁷X⁵,

or a pharmaceutically acceptable salt thereof, wherein:
each X⁵ is independently a charged amino acid (e.g., arginine, histidine, lysine, Dap, Dab, Orn, homo-lysine, homo-arginine, homo-histidine, 4-guanidino-phenylalanine, and 4-amino-phenylalanine);
each X⁶ is independently a modified or unmodified phenylalanine, leucine, isoleucine, tryptophan, or tyrosine; and
each X⁷ is independently a non-charged amino acid (e.g., alanine, valine, leucine, isoleucine, glycine, serine).

In certain embodiments, the promoter peptide comprises the amino acid sequence:

(SEQ ID NO: 5)
X⁵X⁶X⁷X⁵X⁶X⁷X⁵,

or a pharmaceutically acceptable salt thereof, wherein:
each X⁵ is independently selected from arginine, histidine, Dap, Dab, Orn, homo-lysine, homo-arginine, homo-histidine, 4-guanidino-phenylalanine, and 4-amino-phenylalanine;
each X⁶ is independently selected from modified or unmodified phenylalanine, leucine, isoleucine, tryptophan, tyrosine, and norleucine; and
each X⁷ is independently selected from alanine, valine, leucine, isoleucine, glycine, and serine.

In certain embodiments, the promoter peptide comprises the amino acid sequence:

(SEQ ID NO: 6)
X⁵X⁶X⁷X⁵X⁶X⁷X⁵X⁵X⁶X⁷X⁵X⁶X⁷X⁵,

or a pharmaceutically acceptable salt thereof, wherein, X⁵, X⁶, and X⁷ are as defined herein.

In certain embodiments, the promoter peptide comprises the amino acid sequence:

(SEQ ID NO: 19)
X⁵X⁶X⁷X⁵X⁶X⁷X⁵X⁵X⁶X⁷X⁵X⁶X⁷X⁵X⁵X⁶X⁷X⁵,

or a pharmaceutically acceptable salt thereof, wherein X⁵, X⁶, and X⁷ are as defined herein.

In certain embodiments, each X⁵ is independently selected from arginine, histidine, lysine, Dap, Dab, Orn, homo-lysine, homo-arginine, homo-histidine, 4-guanidino-phenylalanine, and 4-amino-phenylalanine. In certain embodiments, each X⁵ is independently selected from arginine, histidine, Dap, Dab, Orn, homo-lysine, homo-arginine, homo-histidine, 4-guanidino-phenylalanine, and 4-amino-phenylalanine. In certain embodiments, each X⁵ is independently selected from lysine, Dap, Dab, Orn, and homo-lysine. In certain embodiments, each X⁵ is independently selected from Dap, Dab, and Orn. In certain embodiments, at least one X⁵ is Dap. In certain embodiments, each X⁵ is Dap.

In certain embodiments, each X⁶ is independently selected from modified or unmodified phenylalanine, leucine, isoleucine, tryptophan, and tyrosine. In certain embodiments, each X⁶ is independently selected from phenylalanine and tyrosine. In certain embodiments, at least one X⁶ is phenylalanine. In certain embodiments, each X⁶ is phenylalanine. In certain embodiments, at least one X⁶ is tyrosine. In certain embodiments, each X⁶ is tyrosine.

In certain embodiments, each X⁷ is independently selected from alanine, valine, leucine, isoleucine, glycine, and serine. In certain embodiments, each X⁷ is independently selected from alanine, glycine, and serine. In certain embodiments, at least one X⁷ is alanine. In certain embodiments, each X⁷ is alanine. In certain embodiments, at least one X⁷ is glycine. In certain embodiments, each X⁷ is glycine. In certain embodiments, at least one X⁷ is serine. In certain embodiments, each X⁷ is serine.

In certain embodiments, the promoter peptide comprises the amino acid sequence: DapFADapFADap (SEQ ID NO: 7), or a pharmaceutically acceptable salt thereof. In certain embodiments, the promoter peptide comprises the amino acid sequence: DapFADapFADapDapFADapFADap (SEQ ID NO: 8), or a pharmaceutically acceptable salt thereof.

In certain embodiments, the promoter peptide comprises the amino acid sequence: DapYADapYADap (SEQ ID NO: 9), or a pharmaceutically acceptable salt thereof. In certain embodiments, the promoter peptide comprises the amino acid sequence: DapYADapYADapDapYADapYADap (SEQ ID NO: 10), or a pharmaceutically acceptable salt thereof.

In certain embodiments, the promoter peptide comprises the amino acid sequence: DapFGDapFGDap (SEQ ID NO: 11), or a pharmaceutically acceptable salt thereof. In certain embodiments, the promoter peptide comprises the amino acid sequence: DapFGDapFGDapDapFGDapFGDap (SEQ ID NO: 12), or a pharmaceutically acceptable salt thereof.

In certain embodiments, the promoter peptide comprises the amino acid sequence: DapFSDapFSDap (SEQ ID NO: 13), or a pharmaceutically acceptable salt thereof. In certain embodiments, the promoter peptide comprises the amino acid sequence: DapFSDapFSDapDapFSDapFSDap (SEQ ID NO: 14), or a pharmaceutically acceptable salt thereof.

In certain embodiments, the promoter peptide comprises one of the following amino acid sequences or a pharmaceutically acceptable salt thereof:

(SEQ ID NO: 15)
(DapFADapFADap)₂DapFADap
(SEQ ID NO: 16)
(DapYADapYADap)₂DapYADap
(SEQ ID NO: 17)
(DapFGDapFGDap)₂DapFGDap
(SEQ ID NO: 18)
(DapFSDapFSDap)₂DapFSDap.

In certain embodiments, the promoter peptide is not a polyarginine. In certain embodiments, the promoter peptide is not TAT (GRKKRRQRRRPQ (SEQ ID NO: 22)). In certain embodiments, the promoter peptide does not comprise KLAK (SEQ ID NO: 23). In certain embodiments, the promoter peptide is not KLAKLAKKLAKLAK (SEQ ID NO: 24).

### Stapled Peptides

In certain embodiments, the conjugates (e.g., PACs) described herein comprise a stapled peptide as the promoter peptide. The terms “stapled” and “crosslinked” are used interchangeably and refer to peptides wherein two amino acids (i.e., “crosslinked amino acids”) are connected via an internal crosslink (i.e., “staple”) to form a macrocycle. The terms “crosslink” and “staple” are used interchangeably and refer to a covalent linking moiety other than the peptide backbone which connects a pair of crosslinked amino acids to form a macrocycle.

Stapled peptide technology is described in, e.g., U.S. Pat. Nos. 7,192,713; 7,786,072; 8,895,699; 9,505,801; 9,951,099; and 10,487,110, the entire contents of each of which is incorporated herein by reference. Other examples of stapled peptide technology can be found in, e.g., International PCT Application Publication Nos. WO 2017/004591, published Jan. 5, 2017; WO 2019/018499, published Jan. 24, 2019; WO 2021/126827, published Jun. 24, 2021; WO 2014/052647, published Apr. 3, 2014; WO 2014/159969, published Oct. 2, 2014; WO 2011/008260, published Jan. 20, 2011; WO 2009/126292, published Oct. 15, 2009; WO 2013/123266, published Aug. 22, 2013; and WO 2021/188659, published Sep. 23, 2021, the entire contents of each of which are incorporated herein by reference. See also, e.g., Mourtada et al., Nature Biotechnology, 2019, vol. 37, 1186-1197.

Stapled peptides of the disclosure include (i) “singly stapled” peptides, meaning peptides including one internal crosslink connecting two crosslinked amino acids; (ii) “doubly stapled” peptides, meaning peptides including two internal crosslinks, each connecting a different pair of crosslinked amino acids; and (iii) “stitched” peptides, meaning peptides including at least two tandem staples, i.e., staples attached to the same crosslinked amino acid. Stapled peptides can include more than two crosslinks (i.e., multiply stapled), with any number of the staples in the stitched configuration.

In certain embodiments, a crosslink is attached to the α-positions of the crosslinked amino acids. In certain embodiments, crosslinked amino acids are separated by 3 amino acids in the amino acid sequence, forming an “i+4 crosslink.” In certain embodiments, crosslinked amino acids are separated by 4 amino acids in the amino acid sequence, forming an “i+5 crosslink.” In certain embodiments, crosslinked amino acids are separated by 6 amino acids in the amino acid sequence, forming an “i+7 crosslink.” In certain embodiments, crosslinked amino acids are separated by 7 amino acids in the amino acid sequence, forming an “i+8 crosslink.”

Stapling (e.g., crosslinking) a peptide can stabilize a secondary structure (e.g., α-helical secondary structure) of the peptide. In certain embodiments, one or more crosslinks of a stapled peptide provided herein stabilize an α-helix of the peptide. In certain embodiments, a peptide has increased α-helicity as compared to a corresponding unstapled (e.g., uncrosslinked) peptide.

A stapled peptide can exhibit α-helical stability by the maintenance of α-helical structure as measured by circular dichroism or NMR. For example, in certain embodiments, the stapled peptide exhibits at least a 1.1, 1.2, 1.25, 1.3, 1.4, 1.5, 1.6, 1.7, 1.75, 1.8, 1.9, or 2-fold increase in α-helicity (e.g., as determined by circular dichroism or NMR) compared to a corresponding unstapled peptide.

In certain embodiments, a stapled peptide provided herein can exhibit about 10%, 15%, 20%, 25%, 30%, 35%, 40%, 45%, 50%, 55%, 60%, 65%, 70%, 75%, 80%, 85%, 90%, 95%, or 100% α-helicity (e.g., as determined by circular dichroism or NMR) compared to a corresponding unstapled peptide.

In certain embodiments, the promoter peptide is a stapled peptide described in, e.g., International PCT Application Publication Nos. WO 2021/126827, published Jun. 24, 2021; WO 2017/004591, published Jan. 5, 2017; and WO 2019/018499, published Jan. 24, 2019; or WO 2022/192664, published Sep. 15, 2022, the entire contents of which is incorporated herein by reference.

### Stapled Magainin Peptides

In certain embodiments, the promoter peptide is a stapled peptide based on the amino acid sequence of a Magainin peptide (e.g., Magainin II). The Magainins are a class of antimicrobial peptides (AMPs) originally found in the African clawed frog (Xenopus laevis). The peptides are cationic, generally lack a stable conformation in water but form amphipathic α-helices in membranes. They are generally known to disrupt the cell membranes of a broad spectrum of cells, including bacteria, protozoa, and fungi. They have also been reported to have anti-cancer activity. The amino acid sequences of the peptides known as “Magainin I” and “Magainin II” are provided below.

Magainin I
(SEQ ID NO: 25)
GIGKFLHSAGKFGKAFVGEIMKS
Magainin II
(SEQ ID NO: 26)
GIGKFLHSAKKFGKAFVGEIMNS

Examples of stapled Magainin peptides, any of which can be used as the promoter peptide component of the conjugates (e.g., PACs) provided herein, can be found in International PCT Application Publication Nos. WO 2017/004591, published Jan. 5, 2017; WO 2019/018499, published Jan. 24, 2019; WO 2021/126827, published Jun. 24, 2021; and WO 2022/192664, published Sep. 15, 2022, the entire contents of each of which are incorporated herein by reference. See also, e.g., Mourtada et al., Nature Biotechnology, 2019, vol. 37, 1186-1197, the entire contents of which is incorporated herein by reference.

In certain embodiments, the promoter peptide is a stapled Magainin peptide. In certain embodiments, the promoter peptide is a stapled Magainin II peptide.

In certain embodiments, the promoter peptide comprises the amino acid sequence:

(SEQ ID NO: 3)
G X¹ G K F X² H S K K K F G K A X³ V G E X⁴,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; and the amino acid sequence includes 0 to 9 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions.

In certain embodiments, the promoter peptide comprises the amino acid sequence:

(SEQ ID NO: 4)
G X¹ G K F X² H S K K K F G K A X³ V G E X⁴ A K K,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; the amino acid sequence includes 0 to 11 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions. In certain embodiments, the amino acid sequence comprises 10 amino acid substitutions. In certain embodiments, the amino acid sequence comprises 11 amino acid substitutions.

In certain embodiments, the promoter peptide comprises the amino acid sequence:

(SEQ ID NO: 1)
G X¹ G Dap F X² Dap Dap Dap Dap Dap F G Dap A X³ V
G E X⁴ A Dap Dap,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; the amino acid sequence includes 0 to 11 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions. In certain embodiments, the amino acid sequence comprises 10 amino acid substitutions. In certain embodiments, the amino acid sequence comprises 11 amino acid substitutions.

In certain embodiments, the promoter peptide comprises the amino acid sequence:

(SEQ ID NO: 2)
G X¹ G Dap F X² Dap Dap Dap Dap Dap FG Dap A X³ V
G E X⁴ A Dap Dap G,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; the amino acid sequence includes 0 to 11 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions. In certain embodiments, the amino acid sequence comprises 10 amino acid substitutions. In certain embodiments, the amino acid sequence comprises 11 amino acid substitutions.

In certain embodiments, a stapled peptide or pharmaceutically acceptable salt thereof comprises one of the following amino acid sequences:

(SEQ ID NO: 27)
G X¹ G K F X² K S K K K F G K A X³ V G E X⁴ A K K
(SEQ ID NO: 20)
G X¹ G K F X² H K K K K F G K A X³ V G E X⁴ A K K
(SEQ ID NO: 21)
G X¹ G K F X² K K K K K F G K A X³ V G E X⁴ A K K
(SEQ ID NO: 28)
G X¹ G S F X² H K K K K F G K A X³ V G E X⁴ A K K
(SEQ ID NO: 29)
G X¹ G K F X² H N K K K F G K A X³ V G E X⁴ A K K
(SEQ ID NO: 30)
G X¹ G K F X² H Q K K K F G K A X³ V G E X⁴ A K K
(SEQ ID NO: 31)
G X¹ G K F X² H T K K K F G K A X³ V G E X⁴ A K K
(SEQ ID NO: 32)
G X¹ G K F X² H Y K K K F G K A X³ V G E X⁴ A K K
(SEQ ID NO: 33)
G X¹ G K F X² H S K K K F G K A X³ V W E X⁴ A K K
(SEQ ID NO: 34)
G X¹ G K F X² H S K K K F G K A X³ V V E X⁴ A K K
(SEQ ID NO: 35)
G X¹ G K F X² H S K K K F G K A X³ V L E X⁴ A K K
(SEQ ID NO: 36)
G X¹ G K F X² H S K K K F G K A X³ V Y E X⁴ A K K
(SEQ ID NO: 37)
G X¹ G K F X² H S K K K F G K A X³ V F E X⁴ A K K
(SEQ ID NO: 38)
G X¹ G K F X² H S K K K F G K A X³ V T E X⁴ A K K
(SEQ ID NO: 39)
G X¹ G K F X² H S K K K F G K A X³ V G D X⁴ A K K
(SEQ ID NO: 40)
G X¹ G K F X² H S K K K F G K A X³ V G Q X⁴ A K K
(SEQ ID NO: 41)
G X¹ G K F X² H S K K K F G K A X³ V G N X⁴ A K K
(SEQ ID NO: 42)
G X¹ G K F X² K S K K K F G K A X³ V V E X⁴ A K K
(SEQ ID NO: 43)
G X¹ G K F X² K S K K K F G K A X³ V F E X⁴ A K K
(SEQ ID NO: 44)
G X¹ G K F X² H K K K K F G K A X³ V V E X⁴ A K K
(SEQ ID NO: 45)
G X¹ G K F X² H K K K K F G K A X³ V F E X⁴ A K K
(SEQ ID NO: 46)
G X¹ G Dab F X² Dab Dab Dab Dab Dab F G Dab A X³ V G E X⁴ A Dab Dab
(SEQ ID NO: 47)
G X¹ G Orn F X² Orn Orn Orn Orn Orn F G Orn A X³ V G E X⁴ A Orn Orn
(SEQ ID NO: 1)
G X¹ G Dap F X² Dap Dap Dap Dap Dap F G Dap A X³ V G E X⁴ A Dap Dap
(SEQ ID NO: 48)
G X¹ G K F X² K K K K K F G K A X³ V G E X⁴ A K K G G E
(SEQ ID NO: 49)
G X¹ G K F X² K K K K K F G K A X³ V G E X⁴ K
(SEQ ID NO: 50)
G X¹ G K F¹ X² H K K K K F¹ G K A X³ V V E X⁴ A K K
(SEQ ID NO: 51)
G X¹ G K F⁴ X² H K K K K F⁴ G K A X³ V V E X⁴ A K K
(SEQ ID NO: 52)
G X¹ G K F⁵ X² H K K K K F⁵ G K A X³ V V E X⁴ A K K
(SEQ ID NO: 53)
G X¹ G K F² X² H K K K K F² G K A X³ V V E X⁴ A K K
(SEQ ID NO: 54)
G X¹ G K F³ X² H K K K K F³ G K A X³ V V E X⁴ A K K
(SEQ ID NO: 55)
G X¹ G K F X² K K K K K F G K A X³ V V E X⁴ A K K G G E
(SEQ ID NO: 56)
G X¹ G K F X² K K K K K F G K A X³ V F E X⁴ A K K G G E
(SEQ ID NO: 57)
G X¹ G K F X² K K K K K F G K A X³ V F¹ E X⁴ A K K G G E
(SEQ ID NO: 58)
G X¹ G K F X² K K K K K F G K A X³ V F² E X⁴ A K K G G E
(SEQ ID NO: 59)
G X¹ G K F X² K K K K K F G K A X³ V F³ E X⁴ A K K G G E
(SEQ ID NO: 60)
G X¹ G Dab F X² K K K K K F G K A X³ V G E X⁴ A K K
(SEQ ID NO: 61)
G X¹ G K F X² K Dab K K K F G K A X³ V G E X⁴ A K K
(SEQ ID NO: 62)
G X¹ G K F X² K K K K K F G Dab A X³ V G E X⁴ A K K
(SEQ ID NO: 63)
G X¹ G K F X² K K Dap K K F G K A X³ V G E X⁴ A K K
(SEQ ID NO: 64)
G X¹ G K F X² Dab K K K K F G K A X³ V G E X⁴ A K K
(SEQ ID NO: 65)
G X¹ G K F X² K K Dab K K F G K A X³ V G E X⁴ A K K
(SEQ ID NO: 66)
G X¹ G K F X² K K K Dab K F G K A X³ V G E X⁴ A K K
(SEQ ID NO: 67)
G X¹ G K F X² K K K K K F G K A X³ V G E X⁴ A Dab K
(SEQ ID NO: 68)
G X¹ G Dap F X² K K K K K F G K A X³ V G E X⁴ A K K
(SEQ ID NO: 69)
G X¹ G K F X² Dap K K K K F G K A X³ V G E X⁴ A K K
(SEQ ID NO: 70)
G X¹ G K F X² K Dap K K K F G K A X³ V G E X⁴ A K K
(SEQ ID NO: 71)
G X¹ G K F X² K K K Dap K F G K A X³ V G E X⁴ A K K
(SEQ ID NO: 72)
G X¹ G K F X² K K K K Dap F G K A X³ V G E X⁴ A K K
(SEQ ID NO: 73)
G X¹ G K F X² K K K K K F G Dap A X³ V G E X⁴ A K K
(SEQ ID NO: 74)
G X¹ G K F X² K K K K K F G K A X³ V G E X⁴ A Dap K
(SEQ ID NO: 75)
G X¹ G K F X² K K K K K F G K A X³ V G E X⁴ A K Dap
(SEQ ID NO: 76)
G X¹ G K F X² K K K K K F G K A X³ V G E X⁴ A K K G E
(SEQ ID NO: 77)
G X¹ G K F X² K K K K K F G K A X³ V G E X⁴ A K K G G G E
(SEQ ID NO: 78)
G X¹ G K F X² K K K K K F G K A X³ V G E X⁴ A K K G G E E
(SEQ ID NO: 2)
G X¹ G Dap F X² Dap Dap Dap Dap Dap F G Dap A X³ V G E X⁴ A Dap Dap G
(SEQ ID NO: 79)
G X¹ G K F X² K K K K K F G K A X³ V G E X⁴ A K K G G Q
(SEQ ID NO: 80)
G X¹ G Dap F X² Dap Dap Dap Dap Dap F G Dap A X³ V G E X⁴ A K K
(SEQ ID NO: 81)
G X¹ G K F X² Dab K K K K F G K A X³ V G E X⁴ A K K
(SEQ ID NO: 82)
G X¹ G K F X² Dap K K K K F G K A X³ V G E X⁴ A K K G G E
(SEQ ID NO: 83)
G X¹ G K F X² K K Dap K K F G K A X³ V G E X⁴ A K K G G E
(SEQ ID NO: 84)
G X¹ G K F X² Dap K Dap K K F G K A X³ V G E X⁴ A K K
(SEQ ID NO: 85)
G X¹ G K F X² Dap K Dap K K F G K A X³ V G E X⁴ A K K G G E
(SEQ ID NO: 86)
J X¹ G Dap F X² Dap Dap Dap Dap Dap F G Dap A X³ V G E X⁴ A Dap Dap
(SEQ ID NO: 87)
G X¹ G K F X² Dap S K K K F G K A X³ V G E X⁴ A K K
(SEQ ID NO: 88)
G X¹ G K F X² Dap K K K K F S K A X³ V G E X⁴ A K K
(SEQ ID NO: 89)
G X¹ G K F X² Dap K K K K F G K S X³ V G E X⁴ A K K
(SEQ ID NO: 90)
G X¹ G K F X² Dap K K K K F G K A X³ V S E X⁴ A K K
(SEQ ID NO: 91)
G X¹ G K F X² Dap K K K K F G K A X³ V G E X⁴ S K K
(SEQ ID NO: 92)
G X¹ G K F X² K S K K K F G K A X³ V G E X⁴ A K K G G E
(SEQ ID NO: 93)
G X¹ G K F X² K K K K K F S K A X³ V G E X⁴ A K K G G E
(SEQ ID NO: 94)
G X¹ G K F X² K K K K K F G K S X³ V G E X⁴ A K K G G E
(SEQ ID NO: 95)
G X¹ G K F X² K K K K K F G K A X³ V S E X⁴ A K K G G E
(SEQ ID NO: 96)
G X¹ G K F X² K K K K K F G K A X³ V G E X⁴ S K K G G E
(SEQ ID NO: 97)
G X¹ G K F X² K K K K K F G K A X³ V G E X⁴ A K K G G E J.

In certain embodiments, the promoter peptide is of SEQ ID NO: 2, or a pharmaceutically acceptable salt thereof. In certain embodiments, the promoter peptide is of SEQ ID NO: 2, or a pharmaceutically acceptable salt thereof, wherein the C-terminus of the peptide is amidated with —NH₂. In certain embodiments, the promoter peptide is of SEQ ID NO: 2, or a pharmaceutically acceptable salt thereof, wherein the C-terminus of the peptide is amidated with —NH₂; and wherein X¹ and X², and X³ and X⁴, are each connected via the crosslink (alk).

In certain embodiments, the promoter peptide comprises one of the following amino acid sequences:

(SEQ ID NO: 98)
X¹ G I G Dap F L X² Dap Dap Dap Dap F G Dap A F V G E I A Dap Dap G
(SEQ ID NO: 99)
X¹ I G Dap F L Dap X² Dap Dap Dap F G Dap A F V G E I A Dap Dap G
(SEQ ID NO: 100)
G X¹ G Dap F L Dap Dap X² Dap Dap F G Dap A F V G E I A Dap Dap G
(SEQ ID NO: 101)
G I X¹ Dap F L Dap Dap Dap X² Dap F G Dap A F V G E I A Dap Dap G
(SEQ ID NO: 102)
G I G X¹ F L Dap Dap Dap Dap X² F G Dap A F V G E I A Dap Dap G
(SEQ ID NO: 103)
G I G Dap X¹ L Dap Dap Dap Dap Dap X² G Dap A F V G E I A Dap Dap G
(SEQ ID NO: 104)
G I G Dap F X¹ Dap Dap Dap Dap Dap F X² Dap A F V G E I A Dap Dap G
(SEQ ID NO: 105)
G I G Dap F L X¹ Dap Dap Dap Dap F G X² A F V G E I A Dap Dap G
(SEQ ID NO: 106)
G I G Dap F L Dap X¹ Dap Dap Dap F G Dap X² F V G E I A Dap Dap G
(SEQ ID NO: 107)
G I G Dap F L Dap Dap X¹ Dap Dap F G Dap A X² V G E I A Dap Dap G
(SEQ ID NO: 108)
G I G Dap F L Dap Dap Dap X¹ Dap F G Dap A F X² G E I A Dap Dap G
(SEQ ID NO: 109)
G I G Dap F L Dap Dap Dap Dap X¹ F G Dap A F V X² E I A Dap Dap G
(SEQ ID NO: 110)
G I G Dap F L Dap Dap Dap Dap Dap X¹ G Dap A F V G X² I A Dap Dap G
(SEQ ID NO: 111)
G I G Dap F L Dap Dap Dap Dap Dap F X¹ Dap A F V G E X² A Dap Dap G
(SEQ ID NO: 112)
G I G Dap F L Dap Dap Dap Dap Dap F G X¹ A F V G E I X² Dap Dap G
(SEQ ID NO: 113)
G I G Dap F L Dap Dap Dap Dap Dap F G Dap X¹ F V G E I A X² Dap G
(SEQ ID NO: 114)
G I G Dap F L Dap Dap Dap Dap Dap F G Dap A X¹ V G E I A Dap X² G
(SEQ ID NO: 115)
G I G Dap F L Dap Dap Dap Dap Dap F G Dap A F X¹ G E I A Dap Dap X².

or a pharmaceutically acceptable salt thereof, wherein:
X¹ and X² are amino acids connected via a crosslink (i.e., crosslinked amino acids); and the amino acid sequence includes 0 to 11 amino acid substitutions, inclusive, at positions other than X¹ and X². In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions. In certain embodiments, the amino acid sequence comprises 10 amino acid substitutions. In certain embodiments, the amino acid sequence comprises 11 amino acid substitutions.

In certain embodiments, a promoter peptide or pharmaceutically acceptable salt thereof provided herein comprises one of the following amino acid sequences:

(SEQ ID NO: 116)
W X¹ G K F X² H S K K K F G K A X³ V G E X⁴ A K K
(SEQ ID NO: 117)
G X¹ W K F X² H S K K K F G K A X³ V G E X⁴ A K K
(SEQ ID NO: 118)
G X¹ G W F X² H S K K K F G K A X³ V G E X⁴ A K K
(SEQ ID NO: 119)
G X¹ G K W X² H S K K K F G K A X³ V G E X⁴ A K K
(SEQ ID NO: 120)
G X¹ G K F X² W S K K K F G K A X³ V G E X⁴ A K K
(SEQ ID NO: 121)
G X¹ G K F X² H W K K K F G K A X³ V G E X⁴ A K K
(SEQ ID NO: 122)
G X¹ G K F X² H S K K K W W K A X³ V G E X⁴ A K K
(SEQ ID NO: 123)
G X¹ G K F X² H S K K K F G K W X³ V G E X⁴ A K K
(SEQ ID NO: 124)
G X¹ G K F X² H S K K K F G K A X³ W G E X⁴ A K K
(SEQ ID NO: 125)
G X¹ G K F X² H S K K K F G K A X³ V W E X⁴ A K K
(SEQ ID NO: 126)
G X¹ G K F X² H S K K K F G K A X³ V G W X⁴ A K K
(SEQ ID NO: 127)
G X¹ G K F X² H S K K K F G K A X³ V G E X⁴ A K W
(SEQ ID NO: 128)
G X¹ G K F X² H S K K K W G K A X³ V G E X⁴ A K K
(SEQ ID NO: 129)
G X¹ G K F X² H S K K K F W K A X³ V G E X⁴ A K K.

In certain embodiments, the promoter peptide comprises the following amino acid sequence:

(SEQ ID NO: 130)
G X¹ G K F X² K K K K K X³ V G E X⁴ A K K,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; and the amino acid sequence optionally includes 0 to 5 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions.

In certain embodiments, the promoter peptide comprises the following amino acid sequence:

(SEQ ID NO: 131)
Z X¹ G K F X² H S A K K F G K A F V,

and pharmaceutically acceptable salts thereof, wherein:
X¹ and X² are independently amino acids (i.e., crosslinked amino acids);
X¹ and X² are connected via a crosslink; and
the amino acid sequence optionally includes 1 to 5 amino acid substitutions, inclusive, at positions other than X¹ and X². In certain embodiments, the amino acid sequence includes 1, 2, 3, 4, or 5 amino acid substitutions. In certain embodiments, the amino acid sequence includes 1 amino acid substitution. In certain embodiments, the amino acid sequence includes 1 or 2 amino acid substitutions. In certain embodiments, the amino acid sequence includes 1 to 3 amino acid substitutions, inclusive. In certain embodiments, the amino acid sequence includes 1 to 4 amino acid substitutions, inclusive. In certain embodiments, the amino acid sequence includes 1 to 5 amino acid substitutions, inclusive.

In certain embodiments, a promoter peptide or a pharmaceutically acceptable salt thereof provided herein comprises one of the following amino acid sequences:

(SEQ ID NO: 132)
Z X¹ G K F X² H S K K K F G K A F V,
(SEQ ID NO: 133)
Z X¹ G K F X² K S K K K F G K A F V,
(SEQ ID NO: 134)
Z X¹ G K F X² K K K K K F G K A F V,
or
(SEQ ID NO: 135)
Z X¹ G K F X² Dap K K K K F G K A F V.

### Unstapled Magainin Peptides

In certain embodiments, the promoter peptide is an unstapled peptide based on the amino acid sequence of a Magainin peptide (e.g., Magainin II). The amino acid sequences of the peptides known as “Magainin I” and “Magainin II” are provided below.

Magainin I
(SEQ ID NO: 25)
GIGKFLHSAGKFGKAFVGEIMKS
Magainin II
(SEQ ID NO: 26)
GIGKFLHSAKKFGKAFVGEIMNS

In certain embodiments, the promoter peptide is an unstapled Magainin peptide. In certain embodiments, the promoter peptide is an unstapled Magainin II peptide.

In certain embodiments, the promoter peptide comprises the amino acid sequence of SEQ ID NO: 26, or a pharmaceutically acceptable salt thereof, optionally comprising 1-11 amino acid substitutions, inclusive. In certain embodiments, the amino acid sequence comprises 1-11 amino acid substitutions, inclusive. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions. In certain embodiments, the amino acid sequence comprises 10 amino acid substitutions. In certain embodiments, the amino acid sequence comprises 11 amino acid substitutions.

In certain embodiments, the promoter peptide is an unstapled peptide comprising the amino acid sequence:

(SEQ ID NO: 1)
G X¹ G Dap F X² Dap Dap Dap Dap Dap F G Dap A X³ V
G E X⁴ A Dap Dap,

or a pharmaceutically acceptable salt thereof, wherein:
X¹, X², X³, and X⁴ are independently amino acids; and the amino acid sequence includes 0 to 11 amino acid substitutions, inclusive. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions. In certain embodiments, the amino acid sequence comprises 10 amino acid substitutions. In certain embodiments, the amino acid sequence comprises 11 amino acid substitutions.

In certain embodiments, the promoter peptide is an unstapled peptide comprising the amino acid sequence:

(SEQ ID NO: 2)
G X¹ G Dap F X² Dap Dap Dap Dap Dap F G Dap A X³
V G E X⁴ A Dap Dap G,

or a pharmaceutically acceptable salt thereof, wherein:
X¹, X², X³, and X⁴ are independently amino acids; and the amino acid sequence includes 0 to 11 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions. In certain embodiments, the amino acid sequence comprises 10 amino acid substitutions. In certain embodiments, the amino acid sequence comprises 11 amino acid substitutions.

In certain embodiments, the promoter peptide is an unstapled peptide comprising one of SEQ ID NOs: 1-4, 20, 21, and 27-135, or a pharmaceutically acceptable salt thereof, wherein X¹, X², X³, and X⁴ are independently amino acids.

### Additional Stapled and Unstapled Magainin Peptides

In certain embodiments, a promoter peptide or a pharmaceutically acceptable salt thereof comprises one of the following amino acid sequences:

(SEQ ID NO: 142)
G X¹ G Dap F X² Dap Dap Dap Dap Dap F
(SEQ ID NO: 143)
G Dap A X³ V G E X⁴ A Dap Dap G
(SEQ ID NO: 144)
G X¹ G Dap F X² Dap Dap Dap Dap Dap F G Dap A
(SEQ ID NO: 145)
Dap Dap Dap Dap Dap F G Dap A X³ V G E X⁴ A Dap Dap G
(SEQ ID NO: 146)
Dap Dap Dap Dap Dap F G Dap A
(SEQ ID NO: 147)
G X¹ G Dap F X² Dap Dap Dap Dap A X³ V G E X⁴ A Dap Dap G
(SEQ ID NO: 148)
G X¹ G A F X² Dap Dap Dap Dap Dap F G A A X³ V G E X⁴ A A A G
(SEQ ID NO: 149)
G X¹ G Dap F X² A A A A A FG Dap A X³ V G E X⁴ A Dap Dap G
(SEQ ID NO: 150)
G X¹ G A F X² A A A A A F G A A X³ V G E X⁴ A A A G
(SEQ ID NO: 151)
G B G Dap F B Dap Dap Dap Dap Dap F G Dap A X³ V G E X⁴ A Dap Dap G
(SEQ ID NO: 152)
G X¹ G Dap F X² Dap Dap Dap Dap Dap F G Dap A B V G E B A Dap Dap G
(SEQ ID NO: 153)
G B G Dap F B Dap Dap Dap Dap Dap F G Dap A B V G E B A Dap Dap G
(SEQ ID NO: 154)
G X¹ G S F X² Dap Dap Dap Dap Dap F G S A X³ V G E X⁴ A S S G
(SEQ ID NO: 155)
G X¹ G Dap F X² S S S S S F G Dap A X³ V G E X⁴ A Dap Dap G
(SEQ ID NO: 156)
G X¹ G S F X² S S S S S F G S A X³ V G E X⁴ A S S G
(SEQ ID NO: 157)
G X¹ G H F X² Dap Dap Dap Dap Dap F G H A X³ V G E X⁴ A H H G
(SEQ ID NO: 158)
G X¹ G Dap F X² H H H H H F G Dap A X³ V G E X⁴ A Dap Dap G
(SEQ ID NO: 159)
G X¹ G H F X² H H H H H F G H A X³ V G E X⁴ A H H G
(SEQ ID NO: 160)
G X¹ G Dap F X² Dap
(SEQ ID NO: 161)
G X¹ G Dap F X² Dap Dap
(SEQ ID NO: 162)
G X¹ G Dap F X² Dap Dap Dap
(SEQ ID NO: 163)
G X¹ G Dap F X² Dap Dap Dap Dap
(SEQ ID NO: 164)
G X¹ G Dap F X² Dap Dap Dap Dap Dap
(SEQ ID NO: 165)
G X¹ G S F X² Dap Dap Dap Dap Dap F
(SEQ ID NO: 166)
G X¹ G Dap F X² H H H H H F
(SEQ ID NO: 167)
G B G Dap F B Dap Dap Dap Dap Dap F
(SEQ ID NO: 168)
G B G S F B Dap Dap Dap Dap Dap F
(SEQ ID NO: 169)
G B G Dap F B H H H H H F
(SEQ ID NO: 170)
G B G Dap F B Dap Dap Dap Dap A B V G E B A Dap Dap G
(SEQ ID NO: 171)
G B G S F B Dap Dap Dap Dap Dap F G S A B V G E B A S S G
(SEQ ID NO: 172)
G B G Dap F B H H H H H F G Dap A B V G E B A Dap Dap G
(SEQ ID NO: 173)
G X¹ G Dap F X² Dap Dap Dap Dap Dap F¹ G Dap A X³ V G E X⁴ A Dap Dap G
(SEQ ID NO: 174)
G X¹ G Dap F X² Dap Dap Dap Dap Dap F¹

wherein X¹, X², X³, and X⁴ are amino acids (i.e., crosslinked amino acids); X¹ and X² are connected via a crosslink, wherein applicable, and X³ and X⁴ are connected via a crosslink, where applicable.

### Stapled Promoter Peptide Crosslinks

As described herein, stapled promoter peptides comprise one or more crosslinks (e.g., staples), wherein each crosslink connects two amino acids (i.e., crosslinked amino acids) to form a macrocycle. In certain embodiments, when an amino acid sequence comprises X¹ and X², X¹ and X² are crosslinked amino acids connected via a crosslink. Likewise, in certain embodiments, when an amino acid sequence comprises X³ and X⁴, X³ and X⁴ are crosslinked amino acids connected via a crosslink. The following embodiments describing stapled peptide crosslinks apply to all stapled peptides described herein, including all amino acid sequences provided herein and variants thereof described herein.

In certain embodiments, the crosslinks are independently attached to the α-positions of the crosslinked amino acids (e.g., α-positions of X¹, X², X³, and X⁴). In certain embodiments, the crosslinks are independently attached to the α-positions of the crosslinked amino acids (e.g., X¹, X², X³, and X⁴), and the crosslinked amino acids are independently α,α-disubstituted amino acids.

In certain embodiments, each crosslink is independently from about 5 Å to about 35 Å in length, inclusive. In certain embodiments, each crosslink is independently from about 5 Å to about 25 Å in length, inclusive (e.g., in the case of i+4 crosslinks). In certain embodiments, each crosslink is independently from about 6 Å to about 22 Å in length, inclusive. In certain embodiments, each crosslink is independently from about 7 Å to about 20 Å in length, inclusive. In certain embodiments, each crosslink is independently from about 8 Å to about 18 Å in length, inclusive. In certain embodiments, each crosslink is independently from about 9 Å to about 17 Å in length, inclusive. each crosslink is independently about 10 Å to about 16 Å in length, inclusive. In certain embodiments, each crosslink is independently from about 11 Å to about 15 Å in length, inclusive. In certain embodiments, each crosslink is independently from about 12 Å to about 14 Å in length, inclusive. In certain embodiments, each crosslink is independently about 5, 6, 7, 8, 9, 10, 11, 12, 13, 14, 15, 16, 17, 18, 19, 20, 21, 22, 23, 24, or 25 Å in length.

In certain embodiments, each crosslink is independently from about 15 Å to about 35 Å in length, inclusive (e.g., in the case of i+7 crosslinks). In certain embodiments, each crosslink is independently from about 17 Å to about 33 Å in length, inclusive. In certain embodiments, each crosslink is independently from about 19 Å to about 31 Å in length, inclusive. In certain embodiments, each crosslink is independently from about 20 Å to about 30 Å in length, inclusive. In certain embodiments, each crosslink is independently from about 22 Å to about 29 Å in length, inclusive. each crosslink is independently about 24 Å to about 28 Å in length, inclusive. In certain embodiments, each crosslink is independently from about 25 Å to about 27 Å in length, inclusive. In certain embodiments, each crosslink is independently about 15, 16, 17, 18, 19, 20, 21, 22, 23, 24, 25, 26, 27, 28, 29, 30, 31, 32, 33, 34, or 35 Ain length.

In certain embodiments, the length of each crosslink is approximately equal to the length of 5 to 25 carbon-carbon and/or carbon-sulfur bonds, inclusive. In certain embodiments, the length of each crosslink is approximately equal to the length of 5, 6, 7, 8, 9, 10, 11, 12, 13, 14, 15, 16, 17, 18, 19, 20, 21, 22, 23, 24, or 25 carbon-carbon and/or carbon-sulfur bonds, inclusive.

In certain embodiments, the length of each crosslink is approximately equal to the length of 5 to 20 carbon-carbon and/or carbon-sulfur bonds, inclusive. In certain embodiments, the length of each crosslink is approximately equal to the length of 5 to 15 carbon-carbon and/or carbon-sulfur bonds, inclusive. In certain embodiments, the length of each crosslink is approximately equal to the length of 5 to 13 carbon-carbon and/or carbon-sulfur bonds, inclusive. In certain embodiments, the length of each crosslink is approximately equal to the length of 6 to 12 carbon-carbon and/or carbon-sulfur bonds, inclusive. In certain embodiments, the length of each crosslink is approximately equal to the length of 7 to 11 carbon-carbon and/or carbon-sulfur bonds, inclusive. In certain embodiments, the length of each crosslink is approximately equal to the length of 8 to 10 carbon-carbon and/or carbon-sulfur bonds, inclusive.

In certain embodiments, the length of each crosslink is approximately equal to the length of 10 to 20 carbon-carbon and/or carbon-sulfur bonds, inclusive. In certain embodiments, the length of each crosslink is approximately equal to the length of 11 to 19 carbon-carbon and/or carbon-sulfur bonds, inclusive. In certain embodiments, the length of each crosslink is approximately equal to the length of 12 to 18 carbon-carbon and/or carbon-sulfur bonds, inclusive. In certain embodiments, the length of each crosslink is approximately equal to the length of 13 to 17 carbon-carbon and/or carbon-sulfur bonds, inclusive. In certain embodiments, the length of each crosslink is approximately equal to the length of 14 to 16 carbon-carbon and/or carbon-sulfur bonds, inclusive.

In certain embodiments, at least one crosslink spans at least one turn of an α-helix of the peptide. In certain embodiments, each crosslink spans at least one turn of an α-helix of the peptide. In certain embodiments, at least one crosslink spans one turn of an α-helix of the peptide. In certain embodiments, each crosslink spans one turn of an α-helix of the peptide.

In certain embodiments, each pair of crosslinked amino acids (e.g., X¹ and X², and X³ and X⁴) are independently connected by a crosslink to form the following formula:

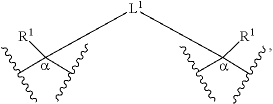

wherein α denotes the α-carbons of the crosslinked amino acids; L¹ is a crosslink; and each instance of R¹ is independently hydrogen or optionally substituted C₁₋₆ alkyl.

In certain embodiments, each crosslink (e.g., L¹) is independently optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

In certain embodiments, each crosslink (e.g., L¹) is independently a hydrocarbon crosslink. “Hydrocarbon crosslink” for the purposes of this disclosure is a crosslink consisting of optionally substituted alkylene, optionally substituted alkenylene, optionally substituted alkynylene, and combinations thereof.

In certain embodiments, each crosslink (e.g., L¹) is independently optionally substituted alkenylene (e.g., unsubstituted alkenylene). In certain embodiments, each crosslink is independently of the following formula:

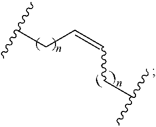

wherein each n is independently an integer from 1-10, inclusive. In certain embodiments, the sum of two n on the same crosslink is 6.

In certain embodiments, the crosslinked amino acids (e.g., X¹, X², X³, and X⁴) are independently α,α-disubstituted amino acids. For instance, in certain embodiments, each pair of crosslinked amino acids (e.g., X¹ and X², and X³ and X⁴) are independently connected by a crosslink to form the following formula:

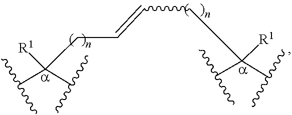

wherein α denotes the α-carbons of the crosslinked amino acids; and wherein each instance of R¹ is independently optionally substituted C₁₋₆ alkyl. In certain embodiments, the sum of two n on the same crosslink is 6.

For example, in certain embodiments, a crosslink (e.g., L¹) is independently of the formula:

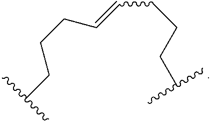 [SMILES: *CCCC=CCCC*]

For example, in certain embodiments, a pair of crosslinked amino acids (e.g., X¹ and X², and X³ and X⁴) are independently connected via a crosslink to form the following formula:

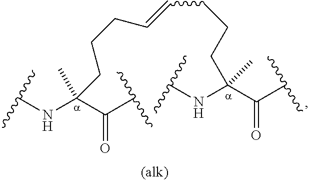 [SMILES: *N[C@@](C)(CCCC=CCCC[C@](C)(N*)C(*)=O)C(*)=O]

wherein α denotes the α-carbons of the crosslinked amino acids.

In certain embodiments, X¹ and X² are connected to form the formula (alk).

In certain embodiments, X³ and X⁴ are connected to form the formula (alk).

In certain embodiments, a crosslink (e.g., L¹) is independently optionally substituted alkylene (e.g., unsubstituted alkylene). In certain embodiments, each crosslink is independently of the following formula:

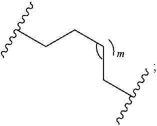

wherein m is an integer from 1-20, inclusive. In certain embodiments, m is 6.

In certain embodiments, a pair of crosslinked amino acids (e.g., X¹ and X², and X³ and X⁴) are independently joined by a crosslink to form the following formula:

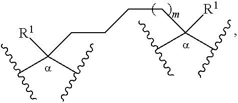

wherein α denotes the α-carbons of the crosslinked amino acids; and wherein each instance of R¹ is independently optionally substituted C₁₋₆ alkyl. In certain embodiments, m is 6.

For example, in certain embodiments, a crosslink (e.g., L¹) is independently of the formula:

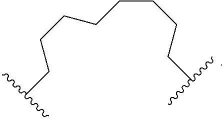 [SMILES: *CCCCCCCC*]

For example, in certain embodiments, a pair of crosslinked amino acids (e.g., X¹ and X², and X³ and X⁴) are connected via a crosslink to form the following formula:

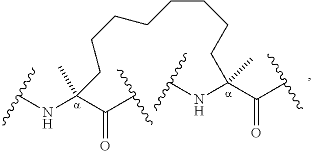 [SMILES: *N[C@@](C)(CCCCCCCC[C@](C)(N*)C(*)=O)C(*)=O]

wherein α denotes the α-carbons of the crosslinked amino acids.

In certain embodiments, a crosslink (e.g., L¹) is independently of the formula:

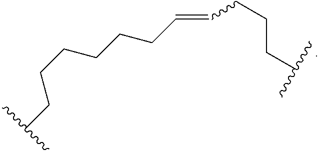 [SMILES: *CCCC=CCCCCCC*]

For example, in certain embodiments, a pair of crosslinked amino acids (e.g., X¹ and X², and X³ and X⁴) are independently connected via a crosslink to form the following formula:

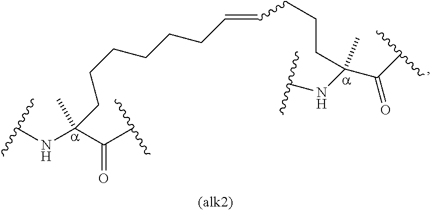 [SMILES: *N[C@@](C)(CCCC=CCCCCCC[C@](C)(N*)C(*)=O)C(*)=O]

wherein α denotes the α-carbons of the crosslinked amino acids.

In certain embodiments, a crosslink (e.g., L¹) is independently of the formula:

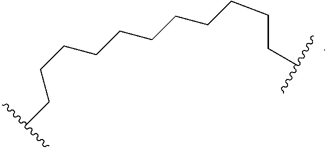 [SMILES: *CCCCCCCCCCC*]

For example, in certain embodiments, a pair of crosslinked amino acids (e.g., X¹ and X², and X³ and X⁴) are independently connected via a crosslink to form the following formula:

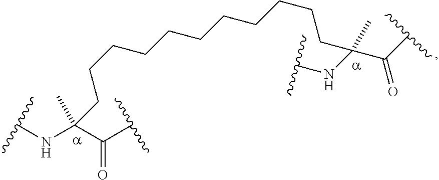 [SMILES: *N[C@@](C)(CCCCCCCCCCC[C@](C)(N*)C(*)=O)C(*)=O]

wherein α denotes the α-carbons of the crosslinked amino acids.

In certain embodiments, a crosslink (e.g., L¹) is independently optionally substituted alkynylene (e.g., unsubstituted alkynylene).

In certain embodiments, a crosslink (e.g., L¹) is independently a dithio crosslink. For the purposes of this disclosure, a “dithio crosslink” (i.e., “dithio staple”) is a crosslink comprising two thioethers (i.e., two —S— groups). In certain embodiments, a crosslink is independently a dithio crosslink of the following formula:

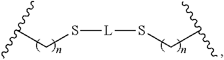

wherein each n is independently an integer from 1-10, inclusive; and
L is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof. In certain embodiments, each instance of n is 1. In certain embodiments, each instance of n is 2.

In certain embodiments, a crosslink is independently a dithio crosslink of the following formula:

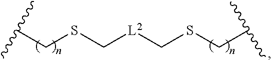

wherein each n is independently an integer from 1-10, inclusive; and
L² is optionally substituted alkylene,

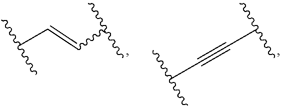

optionally substituted arylene, optionally substituted heteroarylene, or -A¹-A¹-; wherein each instance of A¹ is independently optionally substituted arylene or optionally substituted heteroarylene. In certain embodiments, each instance of n is 1. In certain embodiments, each instance of n is 2.

In certain embodiments, a crosslink is independently a dithio crosslink of one of the following formulae:

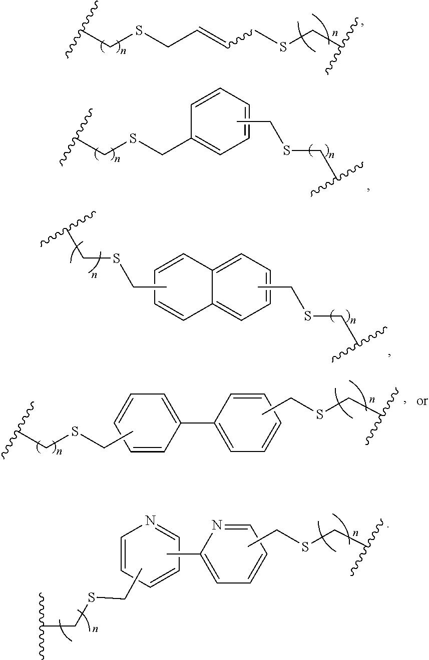

In certain embodiments, each instance of n is 1. In certain embodiments, each instance of n is 2.

For example, in certain embodiments, a crosslink is independently a dithio crosslink of one of the following formulae:

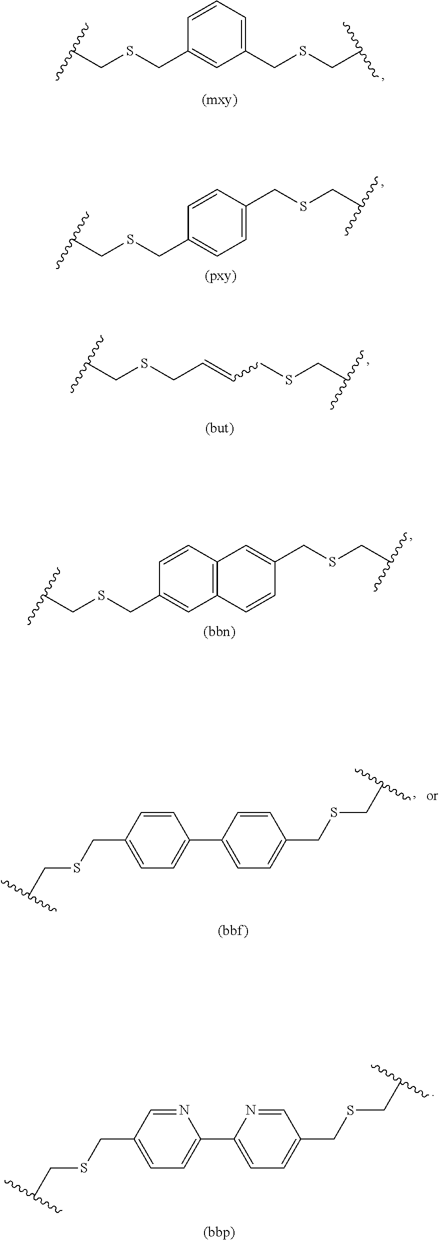 [SMILES: *CSCc1cccc(CSC*)c1 ; *CSCc1ccc(CSC*)cc1 ; *CSCC=CCSC* ; *CSCc1ccc2cc(CSC*)ccc2c1 ; *CSCc1ccc(-c2ccc(CSC*)cc2)cc1 ; *CSCc1ccc(-c2ccc(CSC*)cn2)nc1]

In other embodiments, a crosslink is independently a dithio crosslink of the following formula:

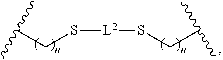

wherein each n is independently an integer from 1-10, inclusive;
L² is an optionally substituted aromatic ring (e.g., a polyhalogenated aryl or heteroaryl ring) or -A¹-A¹-; wherein each instance of A¹ is independently an optionally substituted aromatic ring (e.g., a polyhalogenated aryl or heteroaryl ring). In certain embodiments, each instance of n is 1. In certain embodiments, each instance of n is 2.

In certain embodiments, a crosslink is independently a dithio crosslink of one of the following formulae:

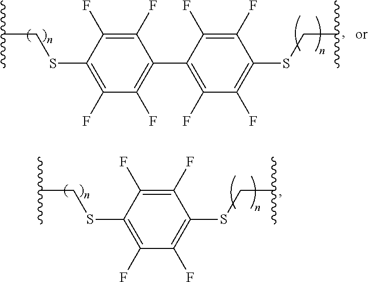

Wherein each n is independently an integer from 1-10, inclusive. In certain embodiments, each instance of n is 1. In certain embodiments, each instance of n is 2.

For example, in certain embodiments, a crosslink is independently of one of the following formulae:

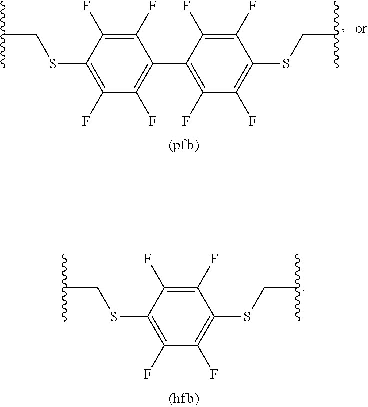 [SMILES: *CSc1c(F)c(F)c(-c2c(F)c(F)c(SC*)c(F)c2F)c(F)c1F ; *CSc1c(F)c(F)c(SC*)c(F)c1F]

For the purposes of this disclosure, the particular crosslinks (mxy), (pxy), (but), (bbn), (bbf), (bbp), (pfb), and (hfb) referenced herein are formed by crosslinking two cysteine (C) residues of a peptide. In other words, a peptide or pharmaceutically acceptable salt thereof provided herein comprising a (mxy), (pxy), (but), (bbn), (bbf), (bbp), (pfb), and/or (hfb) crosslink includes each pair of crosslinked amino acids (e.g., X¹ and X¹, X³ and X⁴) connected via a dithio crosslink to form the following formula:

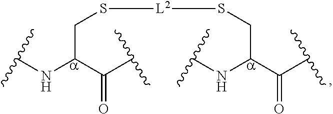

wherein each α represents the alpha-position of a dithio-crosslinked amino acid (e.g., X¹, X², X³, X¹), and L² is as indicated in Table 8 below.

TABLE 8
Certain Dithio Crosslinks

| Dithio Crosslink | L² |
|---|---|
| mxy | 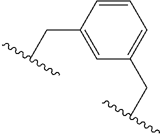 [SMILES: *Cc1cccc(C*)c1] |
| pxy | 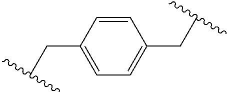 [SMILES: *Cc1ccc(C*)cc1] |
| but | 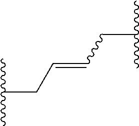 [SMILES: *CC=CC*] |
| bbn | 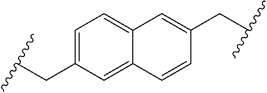 [SMILES: *Cc1ccc2cc(C*)ccc2c1] |
| bbf | 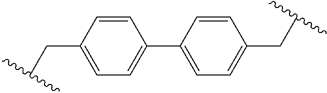 [SMILES: *Cc1ccc(-c2ccc(C*)cc2)cc1] |
| bbp | 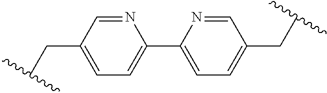 [SMILES: *Cc1ccc(-c2ccc(C*)cn2)nc1] |
| pfb | 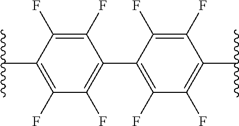 [SMILES: *c1c(F)c(F)c(-c2c(F)c(F)c(*)c(F)c2F)c(F)c1F] |

In certain embodiments, a crosslink (e.g., L¹) is independently a triazolylene crosslink. For the purpose of this disclosure, a “triazolylene crosslink” is a crosslink interrupted by at least one triazolylene moiety

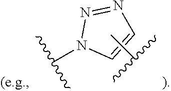

In certain embodiments, a crosslink is independently a triazolylene crosslink of the following formula:

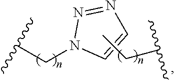

wherein each n is independently an integer from 1-10, inclusive. In certain embodiments, the sum of two n on the same crosslink is 5.

For example, in certain embodiments, a crosslink is independently a triazolylene crosslink of one of the following formulae:

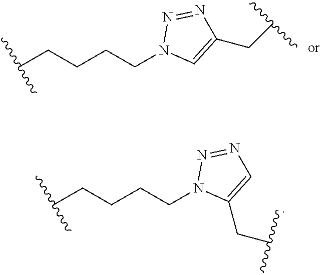 [SMILES: *CCCCn1cc(C*)nn1 ; *CCCCn1nncc1C*]

The following embodiments for n and R¹ apply to all generic formulae and subgenera provided herein, as well as all stapled and unstapled peptides provided herein.

In certain embodiments, the sum of two n on the same crosslink is an integer from 3-9, inclusive. In certain embodiments, the sum of two n on the same crosslink is an integer from 4-8, inclusive. In certain embodiments, the sum of two n on the same crosslink is an integer from 5-7, inclusive. In certain embodiments, the sum of two n on the same crosslink is 5. In certain embodiments, the sum of two n on the same crosslink is 6. In certain embodiments, the sum of two n on the same crosslink is 7.

In certain embodiments, at least one instance of n is 1. In certain embodiments, at least one instance of n is 2. In certain embodiments, at least one instance of n is 3. In certain embodiments, at least one instance of n is 4. In certain embodiments, at least one instance of n is 5. In certain embodiments, at least one instance of n is 6. In certain embodiments, at least one instance of n is 7. In certain embodiments, at least one instance of n is 8. In certain embodiments, at least one instance of n is 9. In certain embodiments, at least one instance of n is 10.

In certain embodiments, m is an integer from 3-9, inclusive. In certain embodiments, m is an integer from 4-8, inclusive. In certain embodiments, m is an integer from 5-7, inclusive. In certain embodiments, m is 5. In certain embodiments, m is 6. In certain embodiments, m is 7.

In certain embodiments, at least one instance of R¹ is hydrogen. In certain embodiments, each instance of R¹ is hydrogen. In certain embodiments, at least one instance of R¹ is unsubstituted C₁₋₆ alkyl. In certain embodiments, at least one instance of R¹ is unsubstituted C₁₋₃ alkyl. In certain embodiments, at least one instance of R¹ is methyl. In certain embodiments, each instance of R¹ is methyl.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Promoter Peptide C- and N-Terminus Modifications

Promoter peptides can include one or more additional modifications anywhere on the peptide (e.g., on an amino acid sidechain, on an α-carbon of an amino acid, on a peptidic nitrogen, at the C-terminus, at the N-terminus, etc.). Promoter peptides can include modifications to the C-terminus and/or N-terminus of the peptide. In certain embodiments, a promoter peptide comprises a modified C-terminus. Examples of C-terminus modifications are described herein.

In certain embodiments, the promoter peptide comprises an amidated C-terminus. Traditionally, peptides comprise a carboxyl group (—C(═O)OH) at the C-terminus. The promoter peptide may comprise an amide at the C-terminus (e.g., —C(═O)NR₂, wherein the group NR₂ is —NH₂, monosubstituted amino, disubstituted amino, or trisubstituted amino), referred to as “amidated C-terminus.” For example, a peptide with a “C-terminus amidated with —NH₂” comprises the group —C(═O)NH₂ at the C-terminus instead of carboxyl (—C(═O)OH). An amidated C-terminus can also be represented by including —NR₂ (e.g., —NH₂) at the end of an amino acid sequence.

Promoter peptides may also be amidated at the C-terminus with an amino acid, peptide, or protein. The amino acid, peptide, or protein can be natural or unnatural. In certain embodiments, the promoter peptide comprises a peptide conjugated to the C-terminus. In certain embodiments, the peptide is from 2 to 6 amino acids in length, inclusive, and comprises amino acids selected from G, E, S, A, and K. In certain embodiments, the peptide is from 2 to 6 amino acids in length, inclusive, and comprises amino acids selected from G, E, and S. In certain embodiments, the peptide is from 2 to 6 amino acids in length, inclusive, and comprises amino acids selected from G and E. In certain embodiments, the peptide is 2 amino acids in length and comprises amino acids selected from G and E. In certain embodiments, the peptide is 3 amino acids in length and comprises amino acids selected from G and E. In certain embodiments, the peptide is 4 amino acids in length and comprises amino acids selected from G and E.

Non-limited examples of peptides which can be conjugated to the C-terminus of the promoter peptide are the following:

GE,
AG,
AA,
GG,
GGE,
GGS,
GGG,
GGK.
GGQ,
(SEQ ID NO: 136)
GGGC
(SEQ ID NO: 137)
GGGE,
(SEQ ID NO: 138)
GGEE,
or
(SEQ ID NO: 139)
GGSGGS.

Promoter peptides may also comprise a small molecule, lipophilic group, or polymer conjugated to the C-terminus of the peptide.

In certain embodiments, the promoter peptide comprises a lipophilic group conjugated to the C-terminus of the peptide. In certain embodiments, the lipophilic group is a lipid or fatty acid. In certain embodiments, the lipophilic group is a hydrocarbon chain.

In certain embodiments, the promoter peptide comprises a polymer conjugated to the C-terminus of the peptide. In certain embodiments, the polymer is a polyether, e.g., polyethylene glycol (PEG). In certain embodiments, the polymer is PEG. In certain embodiments, the polymer is PEG3. As described herein, PEG3 is of the formula:

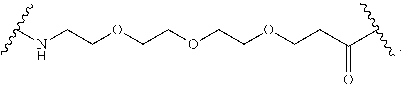 [SMILES: *NCCOCCOCCOCCC(*)=O ; "."]

In certain embodiments, the promoter peptide is amidated at the C-terminus with a group of the following formula: —NH-(PEG)-CONH₂, wherein PEG is polyethylene glycol. In certain embodiments, the promoter peptide is amidated at the C-terminus with a group of the following formula: —NH(CH₂CH₂O)₁₋₂₀CH₂CH₂CONH₂. In certain embodiments, the promoter peptide is amidated at the C-terminus with a group of one of the following formulae: —NHCH₂CH₂OCH₂CH₂CONH₂, —NH(CH₂CH₂O)₂—CH₂CH₂CONH₂, —NH(CH₂CH₂O)₃—CH₂CH₂CONH₂, —NH(CH₂CH₂O)₄—CH₂CH₂CONH₂, or —NH(CH₂CH₂O)₅—CH₂CH₂CONH₂.

In certain embodiments, the promoter peptide comprises a small molecule conjugated to the C-terminus of the peptide. In certain embodiments, the small molecule is an anti-cancer agent.

### Linker and Conjugation

As described herein, a conjugates (e.g., PACs) provided herein comprises a promoter peptide conjugated to an agent (e.g., antibody or antigen-binding fragment thereof). In certain embodiments, the agent (e.g., antibody or antigen-binding fragment thereof) is conjugated to the N-terminus of the promoter peptide. In certain embodiments, the agent (e.g., antibody or antigen-binding fragment thereof) is conjugated to the C-terminus of the promoter peptide. In certain embodiments, the agent (e.g., antibody or antigen-binding fragment thereof) is conjugated to an internal position on the promoter peptide (e.g., to an amino acid residue or to the crosslink of the promoter peptide).

In certain embodiments, PACs described herein comprise antibodies or antigen-binding fragments thereof. In certain embodiments, the promoter peptide is conjugated through a thiol of the antibody or antigen-binding fragment thereof. In certain embodiments, the promoter peptide is conjugated through cysteine residue of the antibody or antigen-binding fragment thereof. In certain embodiments, the promoter peptide is conjugated through an amine of the antibody or antigen-binding fragment thereof. In certain embodiments, the promoter peptide is conjugated through a lysine residue of the antibody or antigen-binding fragment thereof.

In certain embodiments, the agent (e.g., antibody or antigen-binding fragment thereof) is conjugated to the promoter peptide directly (e.g., via a bond). In other embodiments, the agent (e.g., antibody or antigen-binding fragment thereof) is conjugated to the promoter peptide via a linker. “Linker,” as used herein, refers to the moiety linking the agent (e.g., antibody or antigen-binding fragment thereof) to the promoter peptide, not to be confused with the one or more “crosslinks” connecting amino acids of stapled peptides, for example, of the promoter peptide.

In certain embodiments, the linker comprises optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

In certain embodiments, the linker comprises optionally substituted alkylene. In certain embodiments, the linker comprises optionally substituted alkenylene. In certain embodiments, the linker comprises optionally substituted alkynylene. In certain embodiments, the linker comprises optionally substituted heteroalkylene. In certain embodiments, the linker comprises optionally substituted heteroalkenylene. In certain embodiments, the linker comprises optionally substituted heteroalkynylene. In certain embodiments, the linker comprises optionally substituted carbocyclylene. In certain embodiments, the linker comprises optionally substituted heterocyclylene. In certain embodiments, the linker comprises optionally substituted arylene. In certain embodiments, the linker comprises optionally substituted heteroarylene. In certain embodiments, the linker comprises optionally substituted acylene.

In certain embodiments, the linker is a cleavable linker. “Cleavable linker” as used herein refers to a linker capable of cleaving under physiological conditions. In certain embodiments, the linker is pH cleavable or cleavable by a protease, esterase, or intracellular disulfide reduction. In certain embodiments, the linker is cleavable by a protease. See, e.g., Bargh et al., Chem. Soc. Rev. 2019, 48(16), 4361-4374; Zheng Su et al., “Antibody-drug conjugates: Recent advances in linker chemistry”, Acta Pharmaceutica Sinica B, 2021; and Leriche et al., Bioorganic & Medicinal Chemistry, 2012, vol. 20, 571-582, the entire contents of each of which is incorporated herein by reference.

In certain embodiments, the linker is a peptidic linker. In certain embodiments, the linker is a cleavable peptidic linker. “Peptidic linker” as used herein refers to a linker comprising two or more amino acids linked via peptide bonds. In certain embodiments, the peptidic linker comprises —Y^{A}Y^{B}Y^{C}Y^{D}— (SEQ ID NO: 140), wherein:
Y^{A} is glycine, glutamic acid, lysine, or is absent;
Y^{B} is valine, phenylalanine, alanine, tyrosine, or glycine;
Y^{C} is citrulline, arginine, lysine, alanine, or glycine; and
Y^{D} is glycine or is absent.

In certain embodiments, the peptidic linker comprises -GGFG- (SEQ ID NO: 141). In certain embodiments, the peptidic linker comprises -GGG-. In certain embodiments, the peptidic linker comprises -EVC-. See, e.g., Anami et al., Nature Communications, 2018, 9, 2512, the entire contents of which is incorporated herein by reference. In certain embodiments, the peptidic linker comprises -valine-citrulline- (i.e., —V—C—).

Table 6 below shows non-limiting examples of linkers for conjugating a promoter peptide to an antibody or antigen-binding fragment thereof (e.g., in a PAC described herein). The exemplary linkers are shown as the linking reagent and the corresponding resulting linker structure.

TABLE 6
Examples of PAC Linkers and Linking Reagents

| Linker | | Exemplary |
|---|---|---|
| Number | Linker Structure* | Linking Reagent |
| L1 | 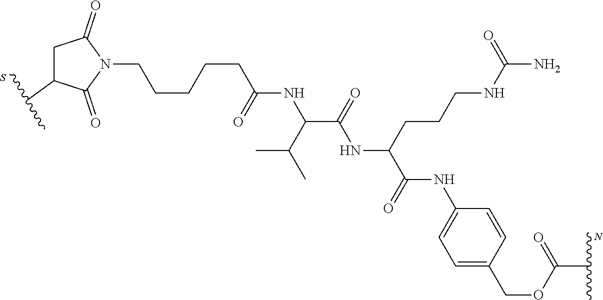 | Maleimidocaproyl-L-valine-L- citrulline-p-aminobenzyl alcohol p-nitrophenyl carbonate (MC-Val-Cit-PABC-PNP) |
| | (maleimide-caproic acid-valine-citrulline-para-aminobenzyl) | |
| L2 | 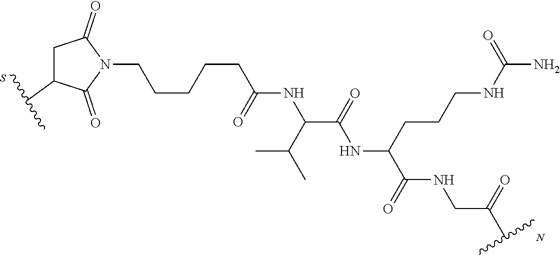 | Maleimidocaproyl- L-valine-L-citrulline-glycine (MC-Val-Cit-Gly) |
| | (maleimide-caproic acid-valine-citrulline-glycine) | |
| L3 | 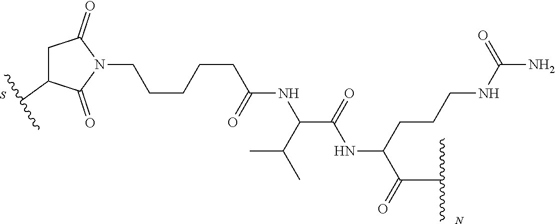 | Maleimidocaproyl- L-valine-L-citrulline (MC-Val-Cit) |
| | (maleimide-caproic acid-valine-citrulline) | |
| L4 | 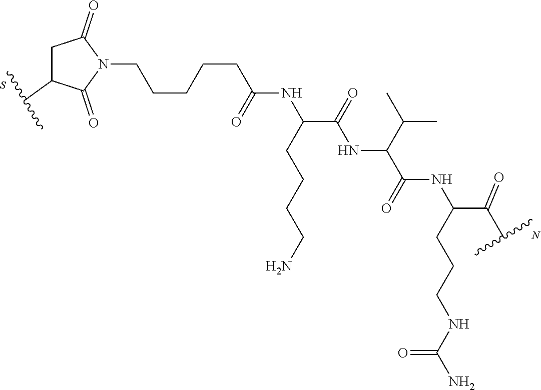 | Maleimidocaproyl- L-lysine-L-valine-L-citrulline (MC-Lys-Val-Cit) |
| | (maleimide-caproic acid-lysine-valine-citrulline) | |
| L5 | 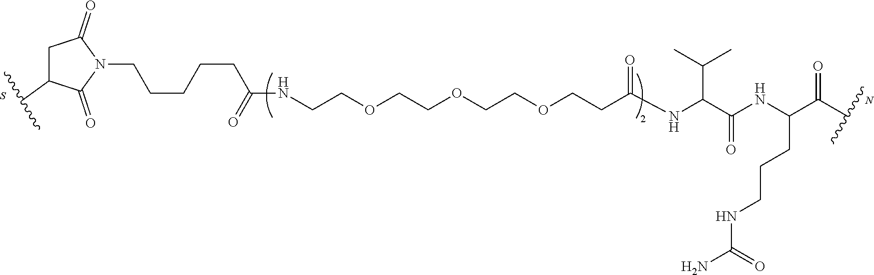 | Maleimidocaproyl- (PEG3)-(PEG3)-L-valine-L-citrulline (MC-(PEG3)- (PEG3)- Val-Cit) |
| | (maleimide-caproic acid-(PEG3)-(PEG3)-valine-citrulline) | |
| L6 | 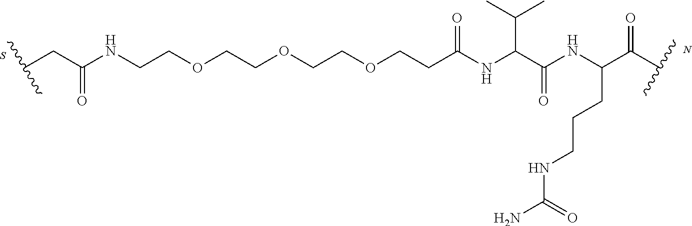 | Iodoacetamide-(PEG3)-L-valine- L-citrulline (IAA-(PEG3)-Val-Cit) or Bromoacetamide- (PEG3)-L-valine- L-citrulline |
| | (acetamido-(PEG3)-valine-citrulline) | |
| L7 | 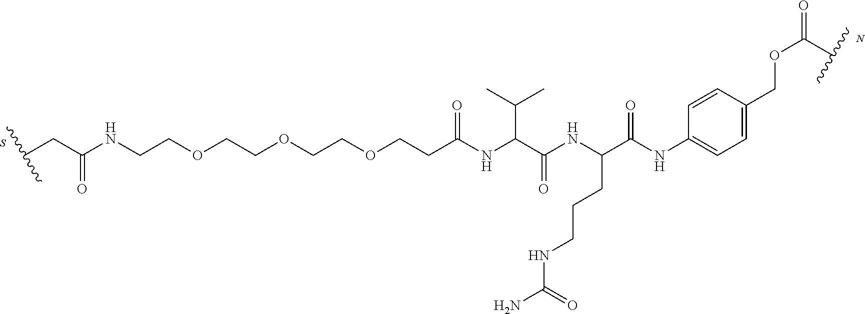 | Iodoacetamide-(PEG3)-L-valine- L-citrulline-p-aminobenzyl alcohol p-nitrophenyl carbonate (IAA-(PEG3)-Val-Cit- PABC-PNP) or Bromoacetamide-(PEG3)-L-valine- L-citrulline-p-aminobenzyl alcohol p-nitrophenyl carbonate |
| | (acetamido-(PEG3)-valine-citrulline-para-aminobenzyl) | |
| L8 | 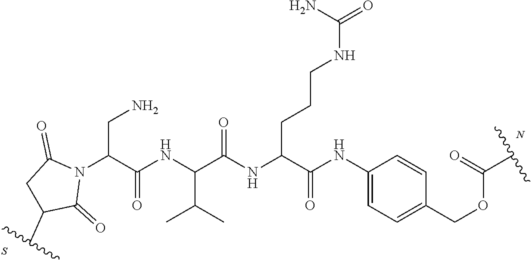 | Maleimido-L-2,3-diaminopropionate- L-valine-L-citrulline-p-aminobenzyl alcohol p-nitrophenyl carbonate |
| | (maleimide-2,3-diaminopropionate-valine-citrulline-para-aminobenzyl) | |
| L9 | 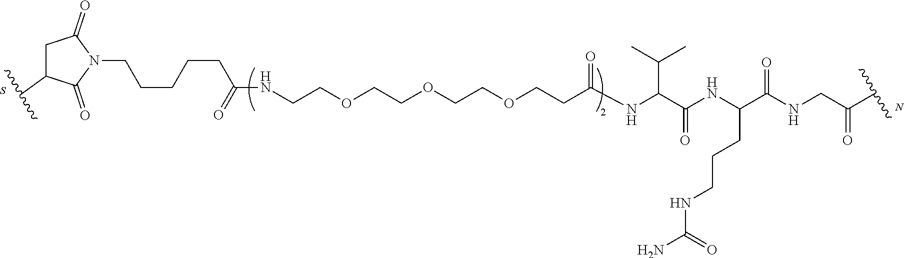 | Maleimidocaproyl- (PEG3)-(PEG3)-L- valine-L-citrulline-glycine (MC-(PEG3)-(PEG3)-Val- Cit-Gly) |
| | (maleimide-caproic acid -(PEG3)2-VC-G) | |
| L10 | 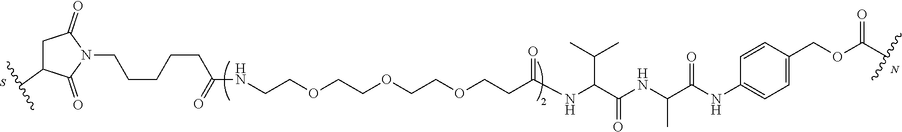 | Maleimidocaproyl-(PEG3)-(PEG3)- L-valine-L-alanine-p-aminobenzyl alcohol p-nitrophenyl carbonate (MC-(PEG3)-(PEG3)-Val- Ala-PABC-PNP) |
| | (maleimide-caproic acid-(PEG3)2-VA-PAB) | |
| L11 | 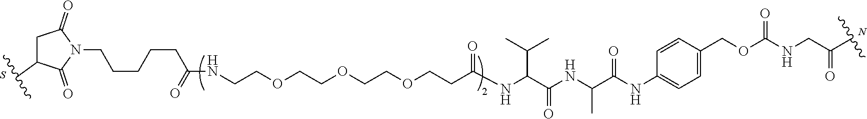 | Maleimidocaproyl- (PEG3)-(PEG3)-L- valine-L-alanine-p- aminobenzyl carbonate-Glycine (MC-(PEG3)-(PEG3)-Val- Ala-PAB-Gly) |
| | (maleimide-caproic acid-(PEG3)2-VA-PAB-G) | |
| L12 | 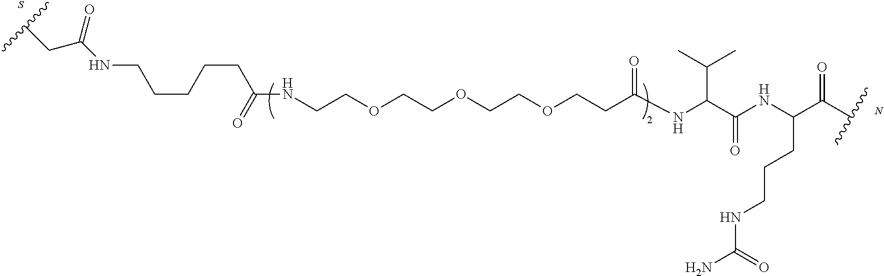 | BrAc-Ahx-PEG3-PEG3-VCit |
| | (acetamidohexanoyl-(PEG3)-(PEG3)-valine-citrulline) | |
| L13 | 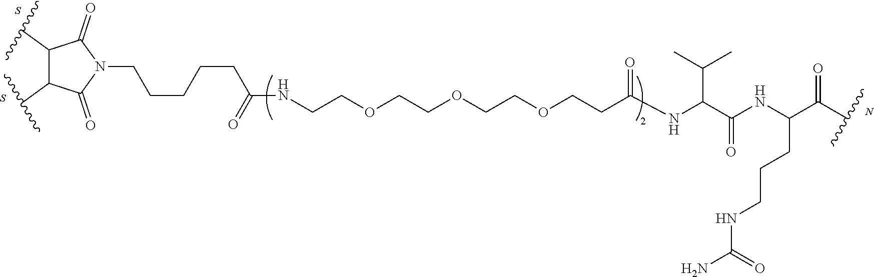 | DBM-Ahx-PEG3-PEG3-Vcit |
| | (dibromomaleimidohexanoyl-(PEG3)-(PEG3)-valine-citrulline) | |
| L14 | 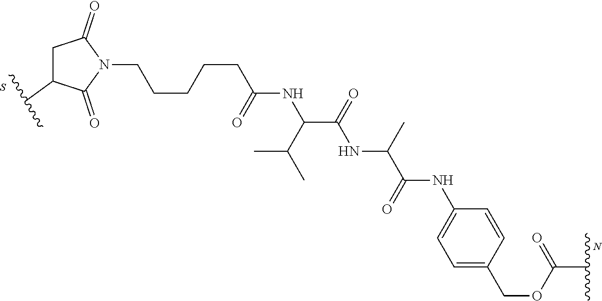 | MC-Val-Ala-PAB |
| | (maleimidocaproyl-valine-alanine-para-aminobenzyl alcohol carbonate ester) | |
| L15 | 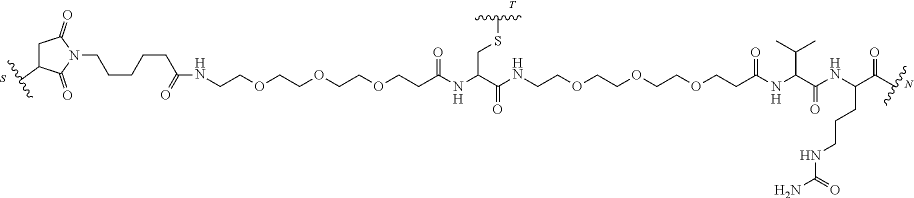 | MC-PEG3- Cys(STMP)-PEG3-VCit |
| | (maleimidocaproyl-(PEG3)-cysteine-(PEG3)-valine-citrulline) | |
| L16 | 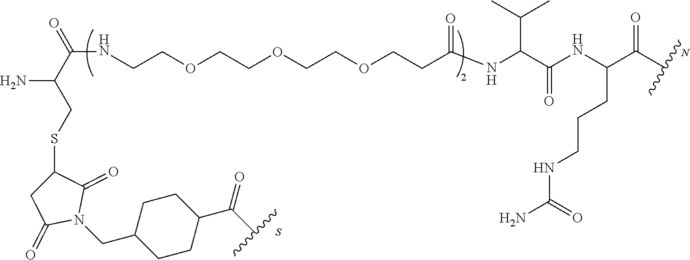 | Succinimidyl 4-(N- maleimidomethyl)c yclohexane-1-carboxylate (SMCC) AND Cys-(PEG3)-(PEG3)-VCit |
| | (4-(N-maleimidomethyl)cyclohexane-1-carboxyl (MCC)-cysteine-(PEG3)-(PEG3)-valine-citrulline) | |
| L17 | 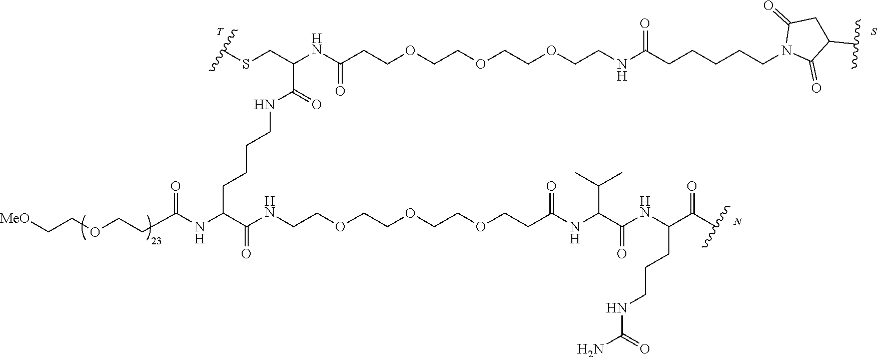 | m-PEG24-K(Cys[STMP]- PEG3-MC)-PEG3-VCit |
| L18 | 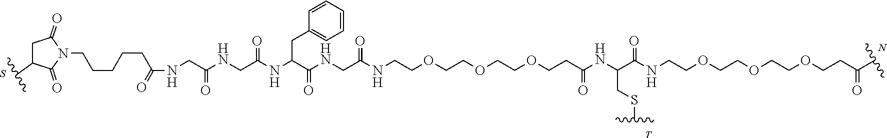 | MC-GGFG-PEG3- Cys(STMP)-PEG3 |
| L19 | 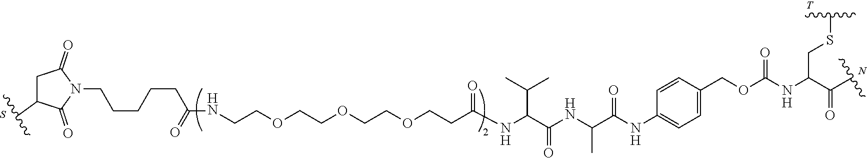 | MC-PEG3-PEG3-VA-PAB- Cys(STMP) |
| L20 | 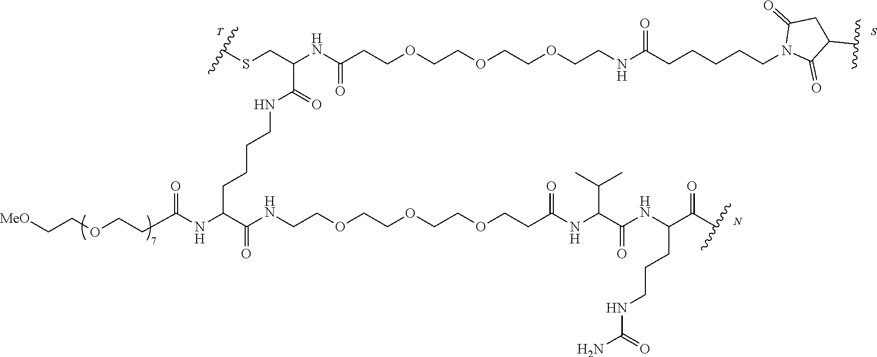 | m-PEG8-K(Cys[STMP]- PEG3-MC)-PEG3-VCit |
| L21 | 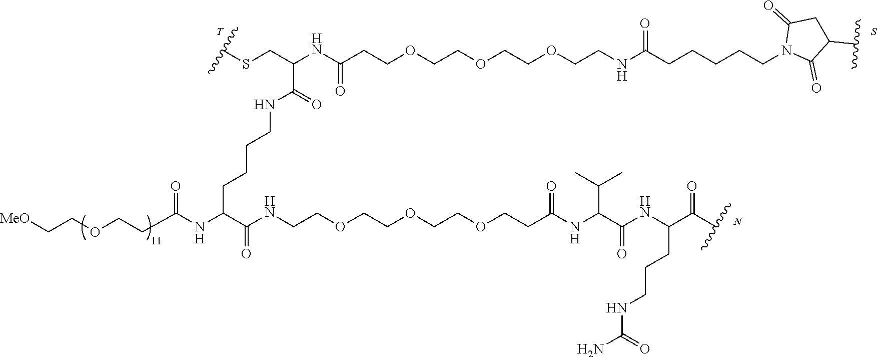 | m-PEG12-K(Cys[STMP]- PEG3-MC)-PEG3-VCit |
| L22 | 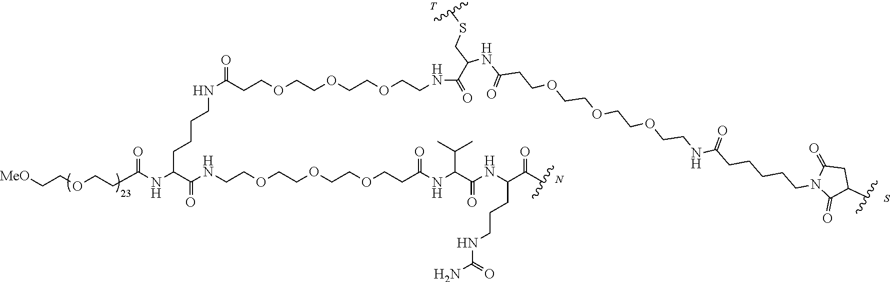 | m-PEG24-K(PEG3- Cys[STMP]-PEG3- MC)-PEG3-VCit |
| L23 | 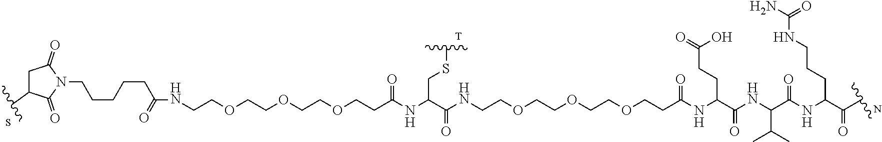 | MC-PEG3-Cys(STMP)-PEG3- EVCit |
| L24 | 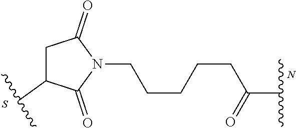 | MC |
| L25 | 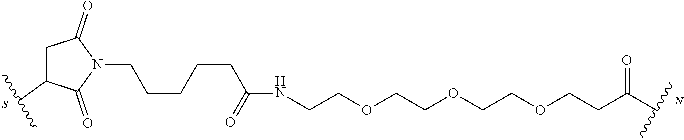 | MC-PEG3 |
| L26 | 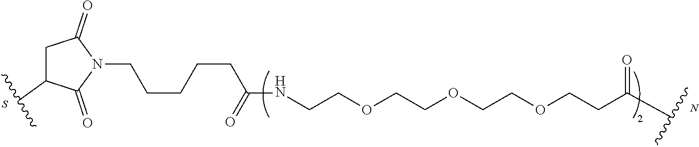 | MC-PEG3-PEG3 |
| L27 | 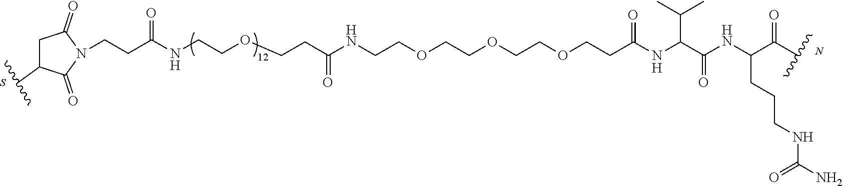 | Mal-PEG12-PEG3-VCit |
| L28 | 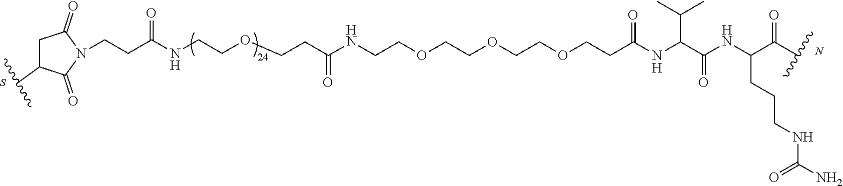 | Mal-PEG24-PEG3-VCit |
| L29 | 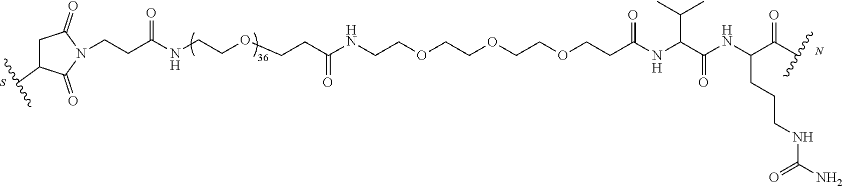 | Mal-PEG36-PEG3-VCit |
| L30 | 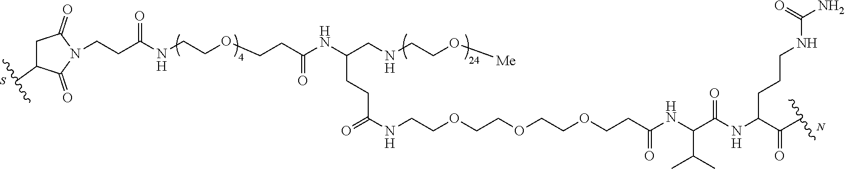 | Mal-PEG4-Glu(PEG3-VCit)- PEG24 |
| L31 | 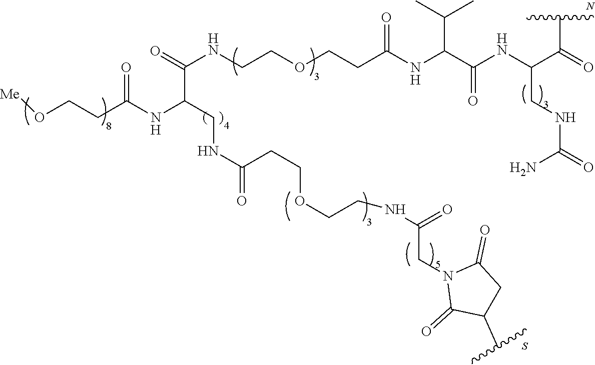 | PEG8-K(PEG3-MC)-PEG3-VCit- OH |
| L32 | 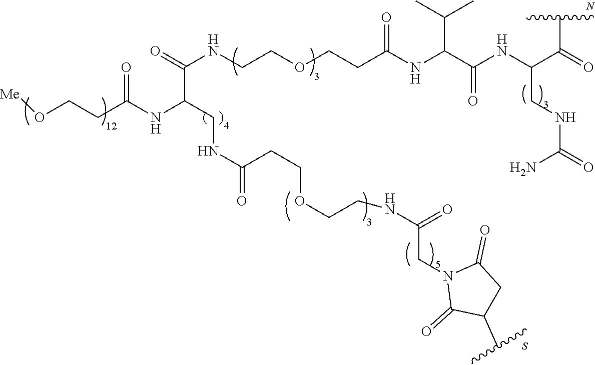 | PEG12-K(PEG3-MC)-PEG3-VCit- OH |
| L33 | 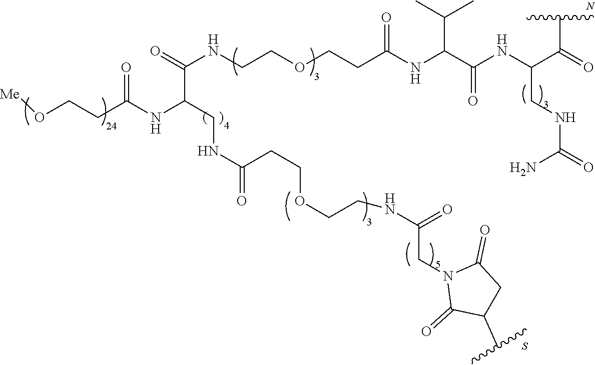 | PEG24-K(PEG3-MC)-PEG3- VCit-OH |
| L34 | 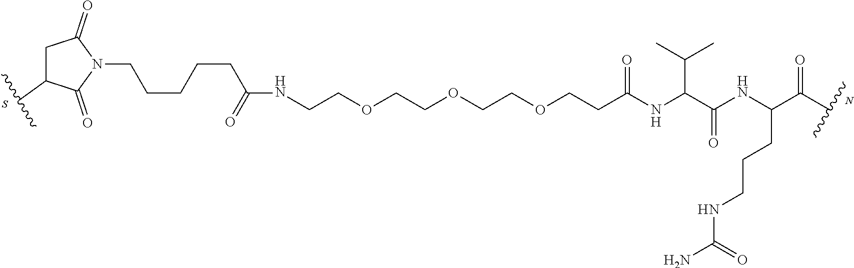 | Maleimidocaproyl- (PEG3)-L-valine- L-citrulline (MC-(PEG3)-Val-Cit) |
| | (maleimide-caproic acid-(PEG3)-valine-citrulline) | |

*In the structures, S denotes the point of attachment to the antibody or antigen-binding fragment thereof; N denotes the point of attachment to the N-terminus of the promoter peptide; and T denotes point of attachment to a cytotoxin or linker-cytotoxin.

In certain embodiments, the linker comprises a triazolylene moiety. As described herein, linkers comprising triazolylene moieties may be formed by azide-alkyne cycloaddition reactions. Non-limiting examples of triazole-containing linkers are shown below in Table 7.

TABLE 7
Examples of PAC Triazolylene-Containing Linkers

| Linker | |
|---|---|
| Number | Linker Structure* |
| A0 | 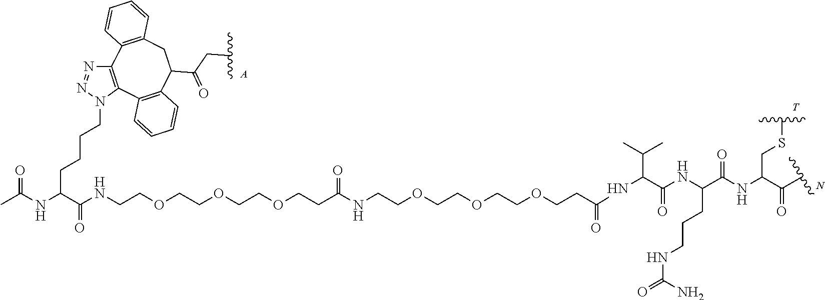 |
| A21 | 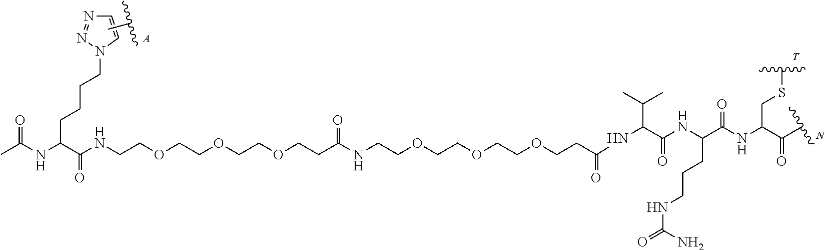 |
| A1 | 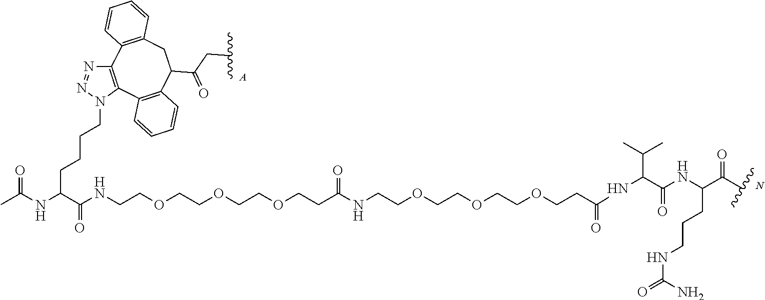 |
| A2 | 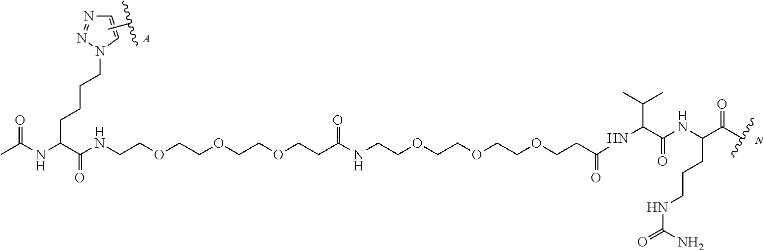 |
| A3 | 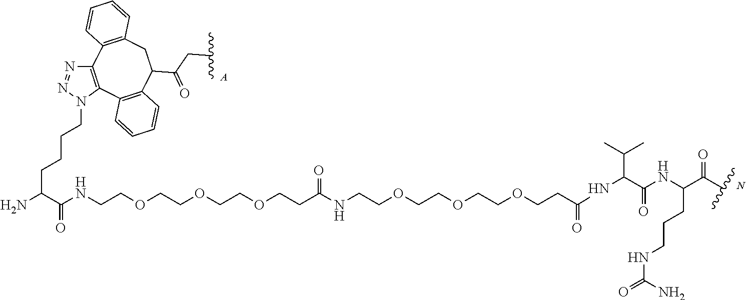 |
| A4 | 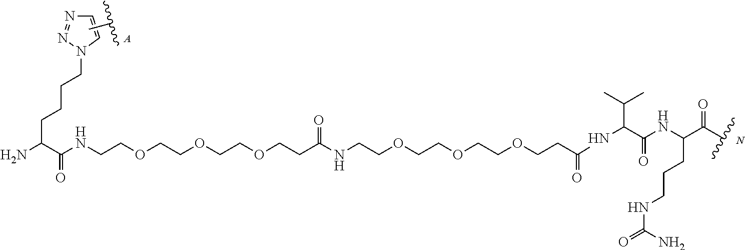 |
| A5 | 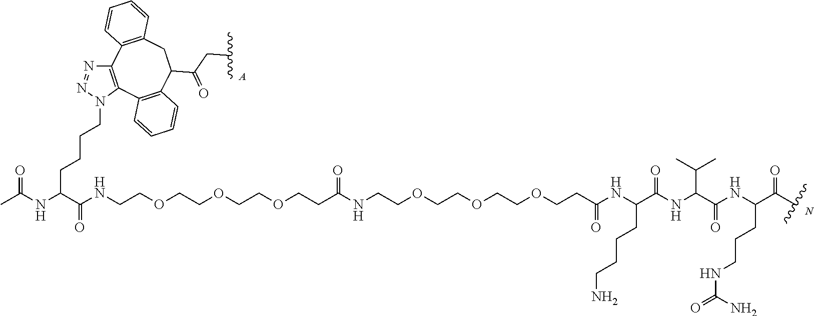 |
| A6 | 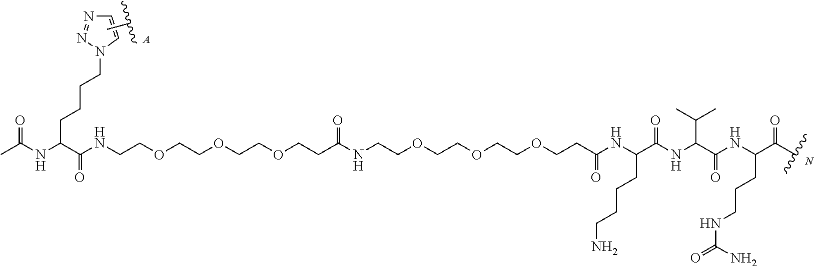 |
| A7 | 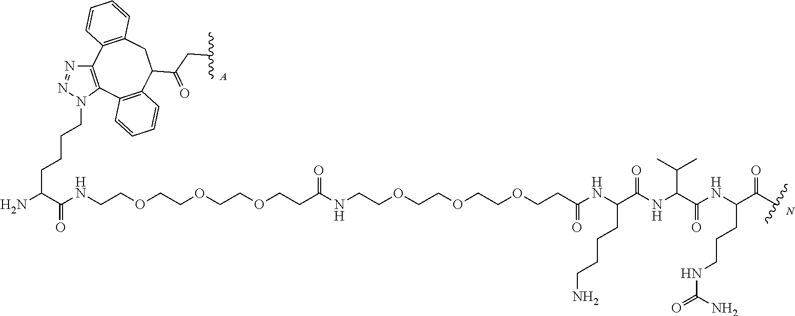 |
| A8 | 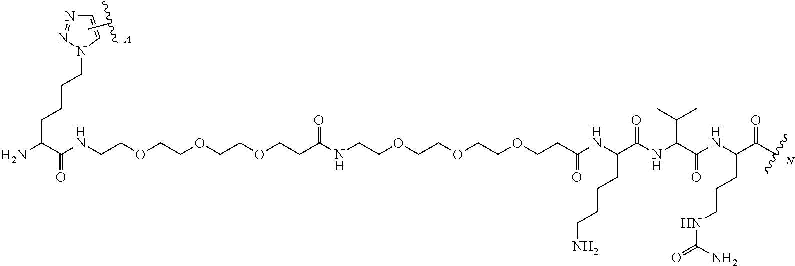 |
| A9 | 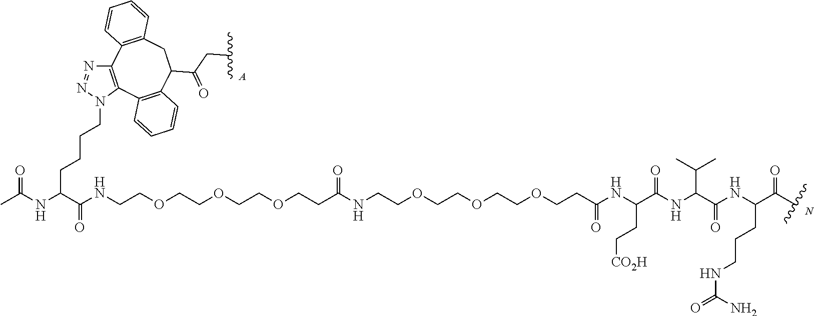 |
| A10 | 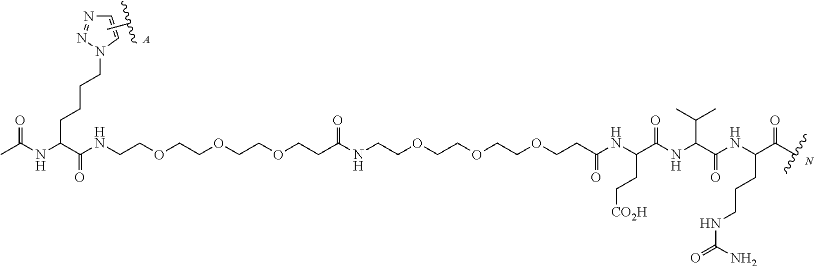 |
| A11 | 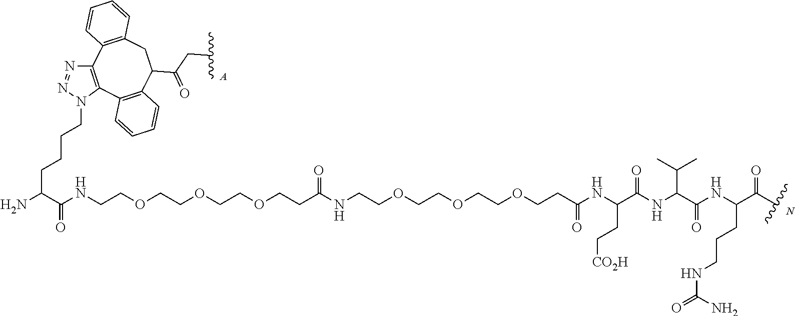 |
| A12 | 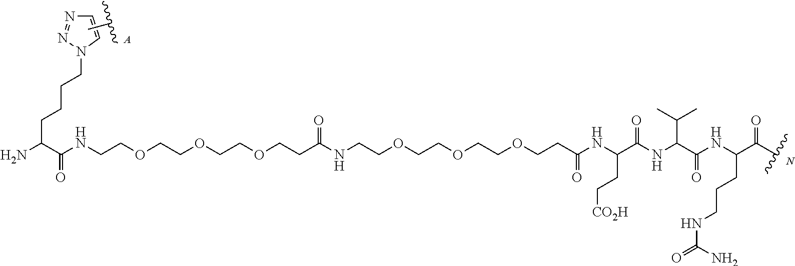 |
| A13 | 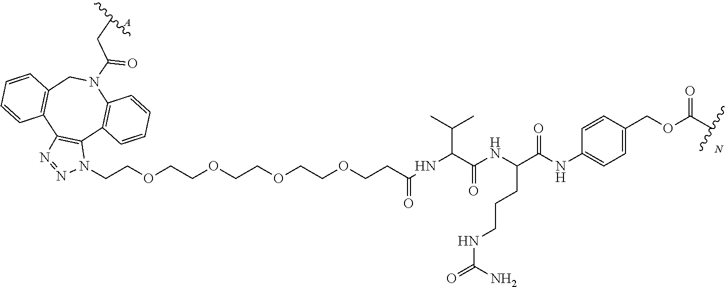 |
| A14 | 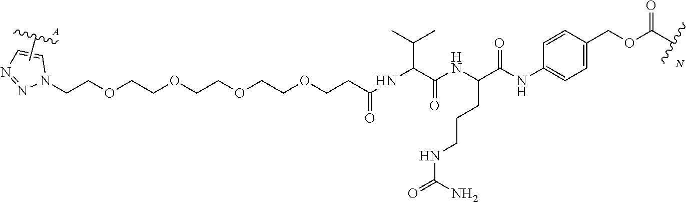 |
| A15 | 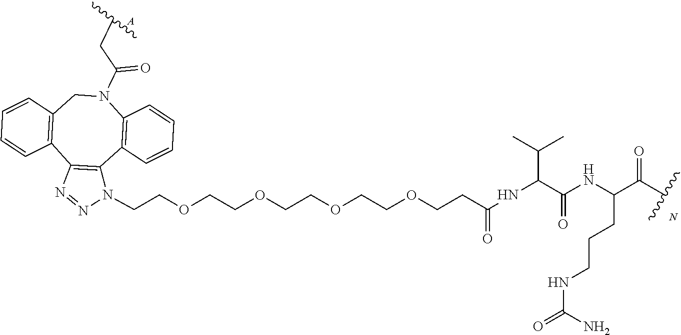 |
| A22 | 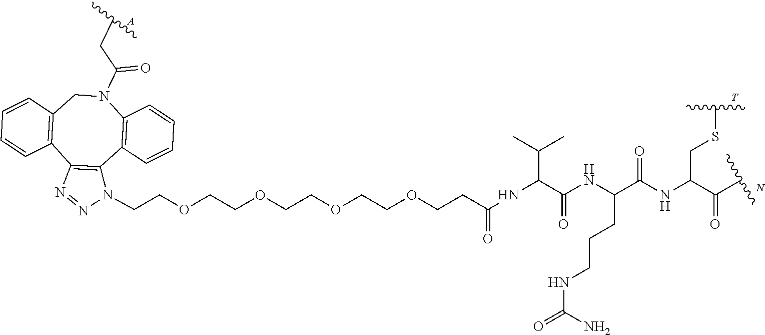 |
| A16 | 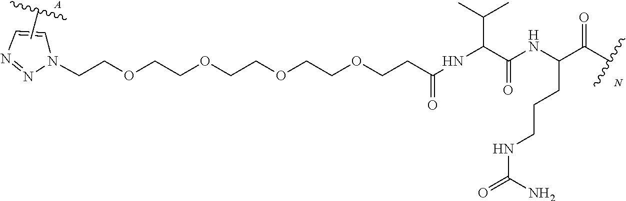 |
| A23 | 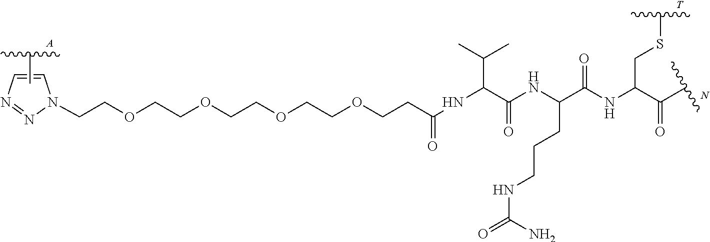 |
| A17 | 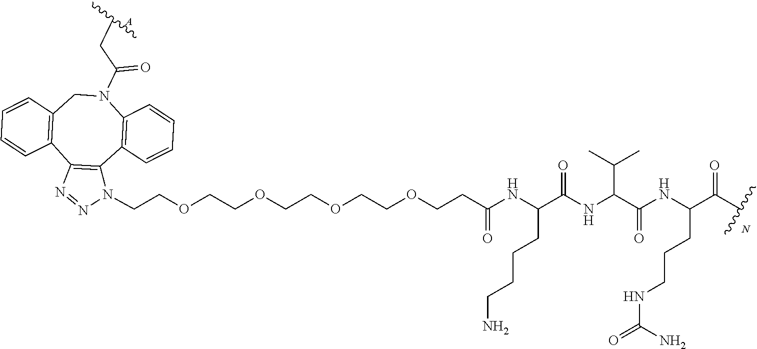 |
| A18 | 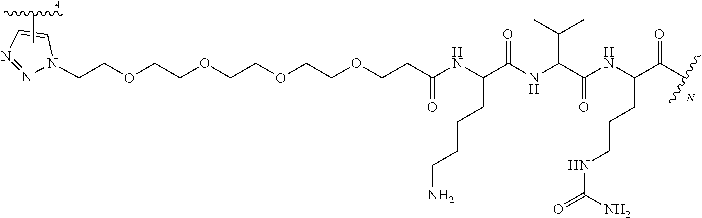 |
| A19 | 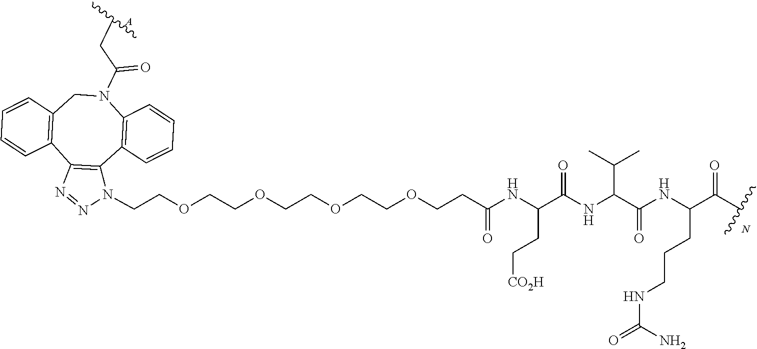 |
| A20 | 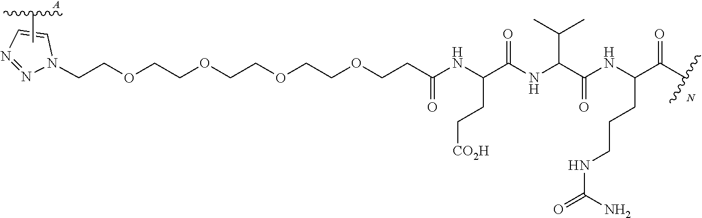 |

*In the structures, A denotes a point of linkage to the antibody or antigen-binding fragment thereof; N denotes the point of attachment to the N-terminus of the promoter peptide; T denotes the point of attachment to an additional agent (e.g., a small molecule, e.g., small molecule cytotoxic agent)

In certain embodiments, a PAC provided herein includes 1 promoter peptide conjugated to the antibody or antigen-binding fragment thereof (i.e., a 1:1 promoter peptide to antibody ratio). In certain embodiments, 2 or more promoter peptides are conjugated to the antibody or antigen-binding fragment thereof (i.e., a 2:1 promoter peptide to antibody ratio or greater). In certain embodiments, 1 to 20 promoter peptides, inclusive, are conjugated to the antibody or antigen-binding fragment thereof (i.e., a 1:1 to 20:1 promoter peptide to antibody ratio, inclusive). In certain embodiments, 2 to 20 promoter peptides, inclusive, are conjugated to the antibody or antigen-binding fragment thereof (i.e., a 2:1 to 20:1 promoter peptide to antibody ratio, inclusive). In certain embodiments, 1 to 10 promoter peptides, inclusive, are conjugated to the antibody or antigen-binding fragment thereof (i.e., a 1:1 to 10:1 promoter peptide to antibody ratio, inclusive). In certain embodiments, 2 to 10 promoter peptides, inclusive, are conjugated to the antibody or antigen-binding fragment thereof (i.e., a 2:1 to 10:1 promoter peptide to antibody ratio, inclusive). In certain embodiments, 5 to 10 promoter peptides, inclusive, are conjugated to the antibody or antigen-binding fragment thereof (i.e., a 5:1 to 10:1 promoter peptide to antibody ratio, inclusive). In certain embodiments, the antibody or antigen-binding fragment thereof is conjugated to 1, 2, 3, 4, 5, 6, 7, 8, 9, 10, 11, 12, 13, 14, 15, 16, 17, 18, 19, or 20 promoter peptides. In certain embodiments, about 8 promoter peptides are conjugated to the antibody or antigen-binding fragment thereof (i.e., an 8:1 promoter peptide to antibody ratio). In certain embodiments, the promoter peptides are the same or different, in any combination.

In certain embodiments, the promoter peptide to antibody ratio is 1:1. In certain embodiments, the promoter peptide to antibody ratio is 2:1. In certain embodiments, the promoter peptide to antibody ratio is 3:1. In certain embodiments, the promoter peptide to antibody ratio is 4:1. In certain embodiments, the promoter peptide to antibody ratio is 5:1. In certain embodiments, the promoter peptide to antibody ratio is 6:1. In certain embodiments, the promoter peptide to antibody ratio is 7:1.

In certain embodiments, a PAC provided herein comprises a 1:1 small molecule payload (e.g., small molecule cytotoxin) to antibody ratio. In certain embodiments, a PAC provided herein comprises a 1:1 to 20:1 small molecule payload (e.g., small molecule cytotoxin) to antibody ratio, inclusive. In certain embodiments, the small molecule payload (e.g., small molecule cytotoxin) to antibody ratio is 1:1 to 10:1, inclusive. In certain embodiments, the small molecule payload (e.g., small molecule cytotoxin) to antibody ratio is 1:1 to 5:1, inclusive. In certain embodiments, the small molecule payload (e.g., small molecule cytotoxin) to antibody ratio is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1.

### Methods of Preparing Peptide-Antibody Conjugates (PACs)

Also provided herein are methods of preparing the peptide-antibody conjugates (PACs) described herein. In certain embodiments, a promoter peptide is conjugated to an antibody or antigen-binding fragment thereof using a linking reagent. “Linking reagent,” as used herein, refers to a molecule comprising two reactive moieties, one capable of reacting with a reactive moiety on the antibody or antigen-binding fragment thereof to form at least one covalent bond, and another capable of reacting with a reactive moiety on the promoter peptide to form at least one covalent bond.

For example, a linking reagent may comprise (i) a moiety capable of reacting with a thiol (e.g., cysteine residue) or amine (e.g., lysine residue) of the antibody or antigen-binding fragment thereof; and (ii) a moiety capable with reacting with the N-terminal amine of the promoter peptide. For example, a linking reagent may comprise (i) a maleimide or iodoacetamide (e.g., capable of reacting with a thiol (e.g., cysteine residue) or amine (e.g., lysine residue) of the antibody or antigen-binding fragment thereof); and (ii) a carboxylic acid or ester (e.g., capable with reacting with the N-terminal amine of the promoter peptide). Non-limiting examples of linking reagents are provided in Table 6.

In certain embodiments, a method of preparing a peptide-antibody conjugate (PAC) described herein comprises the steps of
(a) contacting a promoter peptide with a linking reagent under conditions sufficient to conjugate the linking reagent with the promoter peptide, thereby forming a promoter peptide-linking reagent intermediate; and
(b) contacting the promoter peptide-linking reagent intermediate with an antibody or antigen-binding fragment thereof under conditions sufficient to conjugate the promoter peptide-linking reagent intermediate to the antibody or antigen-binding fragment thereof, thereby forming the peptide-antibody conjugate (PAC).

In certain embodiments, a method for preparing a peptide-antibody conjugate (PAC) described herein comprises the steps of
(a) contacting an antibody or antigen binding fragment thereof with a linking reagent under conditions sufficient to conjugate the linking reagent with antibody or antigen-binding fragment thereof, thereby forming an antibody-linking reagent intermediate; and
(b) contacting the antibody-linking reagent intermediate with a promoter peptide under conditions sufficient to conjugate the antibody-linking reagent intermediate to a promoter peptide, thereby forming a peptide-antibody conjugate (PAC).

In other embodiments, a method for preparing a PAC described herein comprises a step of contacting a promoter peptide comprising a first reactive moiety with an antibody or antigen-binding fragment thereof comprising a second reactive moiety under conditions sufficient to form at least one covalent bond between the first reactive moiety and the second reactive moiety, thereby forming the PAC. In certain embodiments, the first reactive moiety and second reactive moiety are “click chemistry” handles capable of reacting with each other to form one or more covalent bonds therebetween.

“Click chemistry” is a chemical approach introduced by Sharpless in 2001 and describes chemistry tailored to generate substances quickly and reliably by joining small units together. See, e.g., Kolb, Finn, and Sharpless, Angewandte Chemie International Edition (2001) 40: 2004-2021; Evans, Australian Journal of Chemistry (2007) 60: 384-395. Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2]cycloaddition). Examples of alkyne-azide reactions can be found in, e.g., Kolb, Finn and Sharpless Angewandte Chemie International Edition (2001) 40: 2004-2021; Kolb and Sharpless, Drug Discov Today (2003) 24: 1128-1137; and Evans, Australian Journal of Chemistry (2007) 60: 384-395.

In certain embodiments, the first reactive moiety is an azide; and the second reactive moiety is an alkyne. In certain embodiments, the first reactive moiety is an alkyne, and the second reactive moiety is an azide. For example, a method for preparing a PAC described herein in certain embodiments comprises a step of contacting a promoter peptide comprising an azide with an antibody or antigen-binding fragment thereof comprising an alkyne under conditions sufficient to form a triazolylene-containing linker, thereby forming the PAC.

In certain embodiments, the promoter peptide that includes an azide moiety that comprises one of the following formulae* (e.g., to form any one of linkers A0-A23):

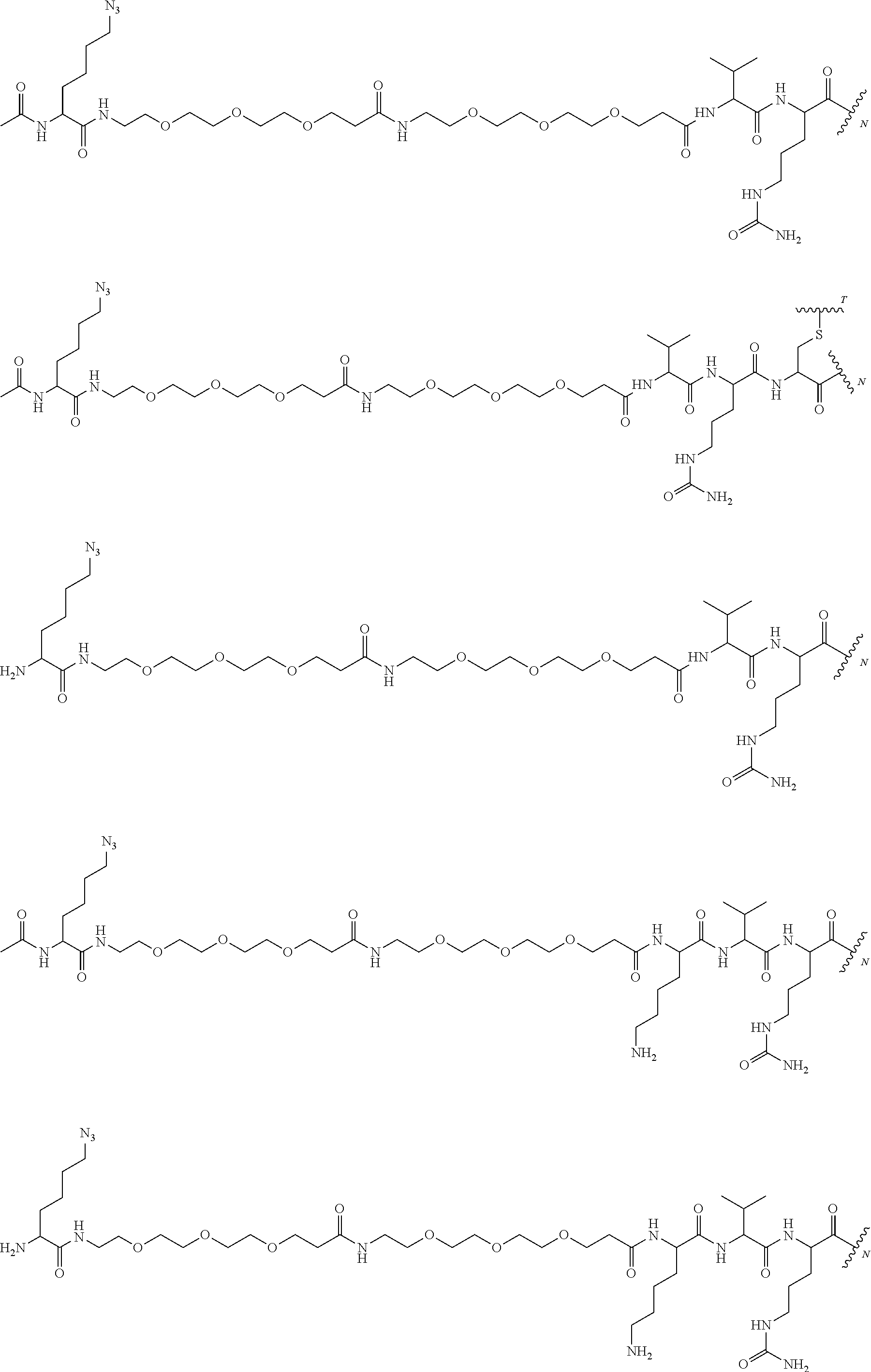

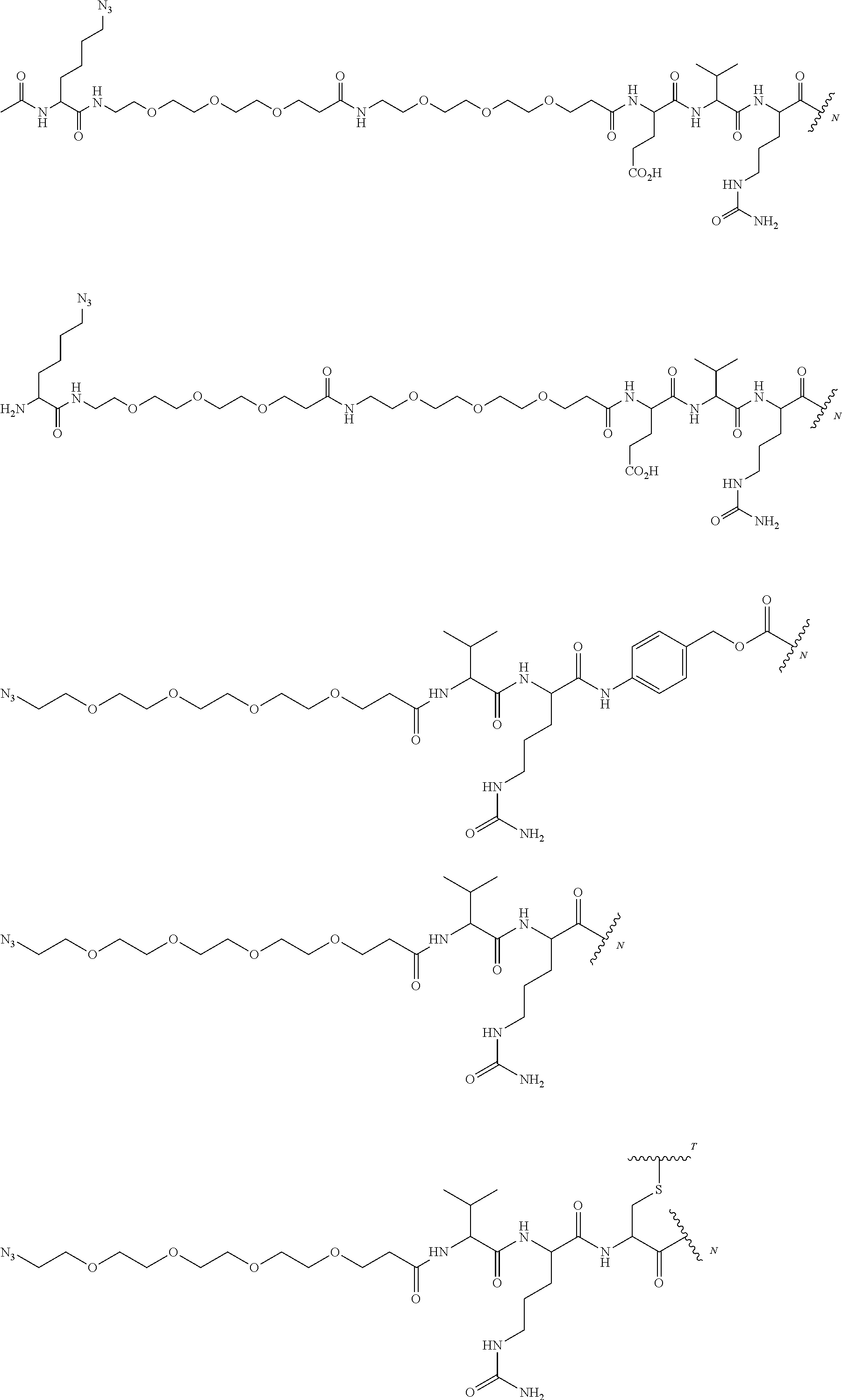

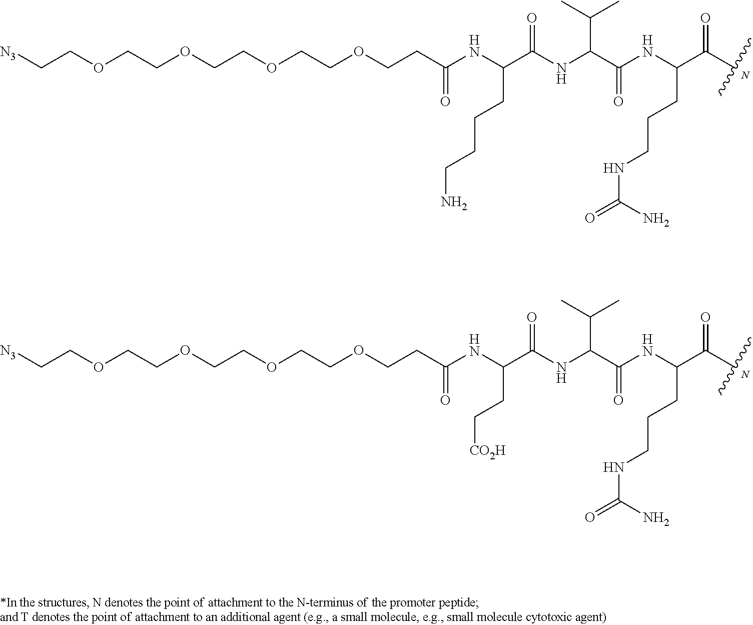

In certain embodiments, the antibody or antigen binding fragment thereof comprises a terminal alkyne (e.g., for use in copper-promoted cycloaddition with an azide). In certain embodiments, the antibody or antigen binding fragment thereof comprises cyclic alkyne (e.g., for use in strain-promoted (e.g., copper-free) cycloaddition with an azide). Non-limiting examples of cyclic alkyne moieties include DBCO and sulfo-DBCO:

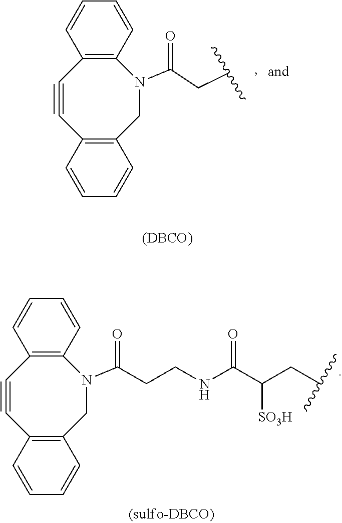

### Pharmaceutical Compositions, Kits, and Administration

The present disclosure provides pharmaceutical compositions comprising a conjugate (e.g., PAC) disclosed herein. The pharmaceutical composition may comprise one or more pharmaceutically acceptable carriers/excipients. In certain embodiments, a conjugate (e.g., PAC) described herein is provided in an effective amount in the pharmaceutical composition. In certain embodiments, the effective amount is a therapeutically effective amount (e.g., for treating cancer in a subject and/or inhibiting tumor growth in a subject). In certain embodiments, the effective amount is a prophylactically effective amount.

Pharmaceutical compositions described herein can be prepared by any method known in the art of pharmacology. In general, such preparatory methods include bringing the conjugate (e.g., PAC) described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient. The composition may comprise between 0.1% and 50% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij© 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In certain embodiments, the formulation comprises a polymer excipient. In certain embodiments, the formulation comprises a polyether. In certain embodiments, the formulation comprises polyethylene glycol (PEG) (e.g., PEG200, PEG300, PEG400, and the like).

Liquid dosage forms for parenteral administration include pharmaceutically acceptable emulsions, microemulsions, solutions, and suspensions. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables. In certain embodiments, the carrier is a buffered aqueous solution.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Conjugates (e.g., PACs) provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The conjugates (e.g., PACs) and compositions provided herein can be administered by any route, including, parenteral, enteral (e.g., oral), intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate a certain route of administration).

The exact amount of a conjugate (e.g., PAC) required to achieve an effective amount will vary from subject to subject, depending, for example, on species, age, and general condition of a subject, severity of the side effects or disorder, identity of the particular conjugate (e.g., PAC), mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a conjugate (e.g., PAC) described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A conjugate (e.g., PAC) or composition, as described herein, can be administered in combination with one or more additional pharmaceutical agents (e.g., therapeutically and/or prophylactically active agents). The conjugates (e.g., PACs) or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a conjugate (e.g., PAC) described herein and an additional pharmaceutical agent shows a synergistic effect that is absent in a pharmaceutical composition including one of the conjugate (e.g., PAC) and the additional pharmaceutical agent, but not both. In some embodiments, the additional pharmaceutical agent achieves a desired effect for the same disorder. In some embodiments, the additional pharmaceutical agent achieves different effects.

The conjugate (e.g., PAC) or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), proteins, carbohydrates, monosaccharides, oligosaccharides, polysaccharides, nucleoproteins, mucoproteins, lipoproteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

The additional pharmaceutical agents include, but are not limited to, anti-proliferative agents, anti-cancer agents, anti-angiogenesis agents, steroidal or non-steroidal anti-inflammatory agents (NSAIDs), immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

Each additional pharmaceutical agent may be administered at a dose and/or on a time schedule determined for that pharmaceutical agent. The additional pharmaceutical agents may also be administered together with each other and/or with the conjugate (e.g., PAC) or composition described herein in a single dose or composition or administered separately in different doses or compositions. The particular combination to employ in a regimen will take into account compatibility of the conjugate (e.g., PAC) described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the conjugate (e.g., PAC) is used in combination with one or more different treatment modalities such as radiation therapy or surgery.

Also encompassed by the disclosure are kits (e.g., pharmaceutical packs). The kits provided may comprise a pharmaceutical composition or conjugate (e.g., PAC) described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or conjugate (e.g., PAC) described herein. In some embodiments, the pharmaceutical composition or conjugate (e.g., PAC) described herein provided in the first container and the second container are combined to form one unit dosage form. Thus, in one aspect, provided are kits including a first container comprising a conjugate (e.g., PAC) or pharmaceutical composition described herein. In certain embodiments, the kits are useful for treating a disease (e.g., cancer) in a subject in need thereof. In certain embodiments, the kits are useful for preventing a disease in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits provide instructions for treating a disease (e.g., cancer) in a subject in need thereof. In certain embodiments, the kits provide instructions for preventing a disease (e.g., cancer) in a subject in need thereof. A kit described herein may include one or more additional pharmaceutical agents described herein as a separate composition.

### Methods of Treatment and Uses

Conjugates (e.g., PACs) provided herein promote the uptake of therapeutically active agents into cells and are therefore useful in the treatment and/or prevention of diseases (e.g., proliferative diseases (e.g., cancer)). Conjugates (e.g., PACs) provided herein can also promote the internalization and/or degradation of cell surface proteins (e.g., target proteins, e.g., receptors) and are therefore useful in the treatment and/or prevention of diseases (e.g., proliferative diseases (e.g., cancer)) associated with said proteins.

Provided herein are methods of treating and/or preventing a disease in a subject comprising administering to the subject a therapeutically and/or prophylactically effective amount of a conjugate (e.g., PAC) provided herein, or a pharmaceutical composition thereof. Also provided herein are conjugates (e.g., PACs), and pharmaceutical compositions thereof, for use in treating and/or preventing a disease in a subject. Also provided herein are uses of conjugates (e.g., PACs), and pharmaceutical compositions thereof, for the manufacture of medicaments. In certain embodiments, the disease is a proliferative disease (e.g., cancer), infectious disease (e.g., bacterial infection), inflammatory disease, or autoimmune disease.

In certain embodiments, the disease is a proliferative disease (e.g., cancer). Provided herein are methods of treating a proliferative disease (e.g., cancer) in a subject comprising administering to the subject a therapeutically effective amount of a conjugate (e.g., PAC) provided herein, or a pharmaceutical composition thereof. Also provided herein are conjugates (e.g., PACs), and pharmaceutical compositions thereof, for use in treating a proliferative disease (e.g., cancer) in a subject. Also provided herein are uses of conjugates (e.g., PACs), and pharmaceutical compositions thereof, for the manufacture of medicaments for treating proliferative diseases (e.g., cancer). In certain embodiments, the proliferative disease is cancer.

A “proliferative disease” refers to a disease that occurs due to abnormal growth or extension by the multiplication of cells (See, e.g., Walker, Cambridge Dictionary of Biology; Cambridge University Press: Cambridge, UK, 1990). A proliferative disease may be associated with: (1) the pathological proliferation of normally quiescent cells; (2) the pathological migration of cells from their normal location (e.g., metastasis of neoplastic cells); (3) the pathological expression of proteolytic enzymes such as the matrix metalloproteinases (e.g., collagenases, gelatinases, and elastases); or (4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasia. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located.

The term “cancer” refers to a class of diseases characterized by the development of abnormal cells that proliferate uncontrollably and have the ability to infiltrate and destroy normal body tissues. In certain embodiments, the cancer is a solid tumor. In certain embodiments, the cancer is a hematopoietic cancer (i.e., hematological cancer).

In certain embodiments, the cancer is a hematopoietic cancer (e.g., leukemia (e.g., acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)); lymphoma (e.g., Hodgkin lymphoma (HL) (e.g., B-cell HL, T-cell HL)), non-Hodgkin lymphoma (NHL) (e.g., B-cell NHL such as diffuse large cell lymphoma (DLCL) (e.g., diffuse large B-cell lymphoma)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome)), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); heavy chain disease (e.g., alpha chain disease, gamma chain disease, mu chain disease); a myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); multiple myeloma (MM); plasma cell neoplasia; familiar hypereosinophilia; inflammatory myofibroblastic tumors; immunocytic amyloidosis). In certain embodiments, the cancer is leukemia. In certain embodiments, the cancer is acute lymphoblastic leukemia (ALL). In certain embodiments, the cancer is early T-cell precursor (ETP)-acute lymphoblastic leukemia (ALL).

In certain embodiments, the cancer is a musculoskeletal cancer (e.g., bone cancer (e.g., osteosarcoma, osteoid osteoma, malignant fibrous histiocytoma, Ewing's sarcoma, chordoma, malignant giant cell tumor chordoma, chondrosarcoma osteochondroma, benign chondroma, chondroblastoma chondromyxofibroma, myelodysplastic syndrome (MDS)), muscle cancer (e.g., rhabdomyosarcoma, rhabdomyoma), connective tissue cancer, synovioma).

In certain embodiments, the cancer is a nervous system cancer (e.g., brain cancer (e.g., astrocytoma, medulloblastoma, glioma (e.g., astrocytoma, oligodendroglioma), glioblastomas, glioblastoma multiform, medulloblastoma, ependymoma, germinoma (i.e., pinealoma), oligodendroglioma, schwannoma, retinoblastoma, congenital tumors, craniopharyngioma), spinal cord cancer, neurofibroma (e.g., neurofibromatosis (NF) type 1 or type 2, schwannomatosis), neuroblastoma, primitive neuroectodermal tumors (PNT), meningeal cancer (e.g., meningioma, meningiosarcoma, gliomatosis), skull cancer, acoustic neuroma, ependymoma, hemangioblastoma, ocular cancer (e.g., intraocular melanoma, retinoblastoma)). In certain embodiments, the disease to be treated is a brain tumor. In certain embodiments, the disease is pleomorphic xenoanthrocytoma (PXA). In certain embodiments, the disease is pediatric pleomorphic xenoanthrocytoma (PXA).

In certain embodiments, the cancer is selected from endocrine/exocrine cancers (e.g., thyroid cancer (e.g., papillary thyroid carcinoma, follicular thyroid carcinoma; medullary thyroid carcinoma, multiple endocrine neoplasia type 2A, multiple endocrine neoplasia type 2B, familial medullary thyroid cancer, pheochromocytoma, paraganglioma), pancreatic cancer (e.g., pancreatic andenocarcinoma, intraductal papillary mucinous neoplasm (IPMN), Islet cell tumors, ductal adenocarcinoma, insulinoma, glucagonoma, vipoma), adrenal gland cancer, neuroendocrine cancer (e.g., gastroenteropancreatic neuroendocrine tumor (GEP-NET), carcinoid tumor), sebaceous gland carcinoma, sweat gland carcinoma). In certain embodiments, the cancer is sweat gland cancer (e.g., sweat gland carcinoma).

In certain embodiments, the cancer is liver cancer (e.g., hepatocellular cancer (HCC) (e.g., hepatocellular carcinoma, hepatoblastoma, hepatocellular adenoma), malignant hepatoma, hemangiomas, biliary cancer (e.g., cholangiocarcinoma)).

In certain embodiments, the cancer is a head and neck cancer (e.g., squamous cell carcinoma of the head and neck (SCCHN), adenoid cystic carcinoma). In certain embodiments, the cancer is oral cancer (e.g., buccal cavity cancer, lip cancer, tongue cancer, mouth cancer, pharynx cancer, hypopharynx cancer (e.g., hypopharyngeal carcinoma), throat cancer (e.g., laryngeal cancer, pharyngeal cancer, nasopharyngeal cancer, oropharyngeal cancer), salivary gland cancer). In certain embodiments, the cancer is esophageal cancer (e.g., esophageal squamous cell carcinoma, esophageal adenocarcinoma, Barrett's adenocarcinoma, esophageal leiomyosarcoma).

In certain embodiments, the cancer is a gastrointestinal cancer (e.g., anal cancer, colorectal cancer (e.g., colon cancer, rectal cancer, colorectal adenocarcinoma), gall bladder cancer, gastric cancer (e.g., stomach cancer (e.g., stomach adenocarcinoma)), gastrointestinal stromal tumor (GIST), small bowel cancer (e.g., appendix cancer, small bowel carcinoma, e.g., small bowel adenocarcinoma), small intestine cancer, large bowel cancer, large intestine cancer).

In certain embodiments, the cancer is cardiovascular cancer (e.g., primary cardiac tumors, angiosarcoma (e.g., lymphangiosarcoma, lymphangioendotheliosarcoma, hemangiosarcoma), endotheliosarcoma (e.g., Kaposi's sarcoma, multiple idiopathic hemorrhagic sarcoma), cardiac myxoma, cardiac rhabdomyoma).

In certain embodiments, the cancer is a lung cancer (e.g., bronchus cancer (e.g., bronchogenic carcinoma, bronchial adenoma), alveolar carcinoma, mesothelioma, small cell lung cancer (SCLC), non-small cell lung cancer (NSCLC), lung adenocarcinoma, chondromatous hamartoma, papillary adenocarcinoma).

In certain embodiments, the cancer is a genitourinary cancer (e.g., bladder cancer (e.g., urothelial carcinoma), urethral cancer, kidney cancer (e.g., nephroblastoma a.k.a. Wilms' tumor, renal cell carcinoma), testicular cancer (e.g., seminoma, testicular embryonal carcinoma), germ cell cancer, prostate cancer (e.g., prostate adenocarcinoma), penile cancer (e.g., Paget's disease of the penis and scrotum)).

In certain embodiments, the cancer is a gynecological cancer (e.g., endometrial cancer (e.g., uterine cancer (e.g., uterine sarcoma, choriocarcinoma), endometrial carcinoma), cervical cancer (e.g., cervical adenocarcinoma), ovarian cancer (e.g., cystadenocarcinoma, ovarian embryonal carcinoma, ovarian adenocarcinoma), germ cell cancer, vulvar cancer (e.g., Paget's disease of the vulva) vaginal cancer, fallopian tube cancer).

In certain embodiments, the cancer is breast cancer (e.g., adenocarcinoma of the breast, papillary carcinoma of the breast, mammary cancer, medullary carcinoma of the breast, triple negative breast cancer, HER-2 positive breast cancer, HER2-negative breast cancer).

In certain embodiments, the cancer is skin cancer (e.g., squamous cell carcinoma (SCC), keratoacanthoma (KA), melanoma, basal cell carcinoma (BCC), dermatofribroma).

In certain embodiments, the cancer is a soft tissue cancer (e.g., intraepithelial neoplasms, epithelial carcinomas, epithelial sarcomas, adenocarcinomas, adenomas, fibrosarcomas, fibromas, liposarcomas, lipomas, myxomas, teratomas).

In certain embodiments, the cancer is a rare cancer. The term “rare cancer” refers to cancers that occur in a relatively small number of patients. Rare cancers include, but are not limited to, sarcomas (e.g., soft tissue sarcoma, liposarcoma, uterine sarcoma, leiomyosarcoma, myxofibrosarcoma, osteosarcoma, angiosarcoma, Ewing's sarcoma, synovial sarcoma, rhabdomyosarcoma, intimal sarcoma), malignant lymphomas, thymic cancer (e.g., thymomas), mesothelioma, gastrointestinal stromal tumors (GISTs), neuroendocrine cancer, eye cancer, brain tumors, bone soft tissue tumors, skin cancer, and germ cell tumors.

In certain embodiments, the cancer is lung cancer (e.g., NSCLC), ovarian cancer, cervical cancer, pancreatic cancer, breast cancer, stomach cancer, esophageal cancer, head and neck cancer, colorectal cancer, multiple myeloma, Leukemia (e.g., chronic lymphocytic leukemia), or lymphoma (e.g., DLBCL, Hodgkin's lymphoma, non-Hodgkin's lymphoma, Burkitt's lymphoma, follicular lymphoma).In certain embodiments, the cancer expresses an antigen selected from CD38, CD20, HER2, and EGFR. In certain embodiments, the cancer expresses said antigen, and the antibody component of the conjugate (e.g., PAC) is an antibody directed against said antigen, or an antigen-binding fragment thereof.

Additionally, provided herein are methods of inhibiting tumor growth in a subject comprising administering to the subject an effective amount of a conjugate (e.g., PAC) provided herein, or a pharmaceutical composition thereof. Also provided herein are conjugates (e.g., PACs), and pharmaceutical compositions thereof, for use in inhibiting tumor growth in a subject. Also provided herein are uses of conjugates (e.g., PACs) provided herein, and pharmaceutical compositions thereof, for the manufacture of medicaments for inhibiting tumor growth.

As used herein the term “inhibit” or “inhibition” in the context of tumor growth, for example, refers to a reduction in the rate of growth of the tumor (i.e., reduction in the rate of proliferation of the tumor's cells). In some embodiments, the term refers to a reduction in the rate of tumor growth to a level that is statistically significantly lower than an initial rate (e.g., the rate of tumor growth before administration or application of a conjugate (e.g., PAC) provided herein). In some embodiments, the term refers to a reduction in the rate of tumor growth to a rate that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial rate (e.g., the rate of tumor growth before administration or application of a conjugate (e.g., PAC) provided herein).

In certain embodiments, treating cancer and/or inhibiting tumor growth can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement. In certain embodiments, the tumor size is reduced by at least 25% relative to its size prior to treatment.

In certain embodiments, treating cancer and/or inhibiting tumor growth may further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×).

In certain embodiments, treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

In certain embodiments, treating cancer can result in an increase in average survival time of a population of subjects treated according to the present disclosure in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the present disclosure. An increase in average survival time of a population may also be measured, for example, by calculating for a population the average length of survival following completion of a first round of treatment with the compound of the present disclosure.

In certain embodiments, treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with the compound of the present disclosure. A decrease in the mortality rate of a population may also be measured, for example, by calculating for a population the average number of disease-related deaths per unit time following completion of a first round of treatment with the compound of the present disclosure.

In certain embodiments, treating cancer can also result in an increased average progression-free survival time of a population of treated subjects in comparison to an untreated population. For example, the average progression-free survival time is increased by more than 30 days (more than 60 days, 90 days, or 120 days). An increase in average progression-free survival time of a population may be measured by any reproducible means. An increase in average progression-free survival time of a population may be measured, for example, by calculating for a population the average length of progression-free survival following initiation of treatment with the compound of the present disclosure. An increase in average progression-free survival time of a population may also be measured, for example, by calculating for a population the average length of progression-free survival following completion of a first round of treatment with the compound of the present disclosure. “Progression-free survival” as used herein refers to the length of time during and after medication or treatment during which the disease being treated (e.g., cancer) does not get worse.

Also provided herein are methods of triggering cancer cell death comprising contacting the cancer cell with an effective amount of a conjugate (e.g., PAC) provided herein, or a pharmaceutical composition thereof. In certain embodiments, the method is a method for selectively triggering cancer cell death (i.e., selectively killing cancer cells). In certain embodiments, a conjugate (e.g., PAC) provided herein is selectively cytotoxic to cancer cells. In certain embodiments, the cell is contacted in vitro. In certain embodiments, the cell is contacted in vivo (i.e., in a subject). In certain embodiments, the cell is contacted in a biological sample.

A conjugate (e.g., PAC) described herein “selectively” triggers the death of one type of cell over another (e.g., selectively triggers cancer cell death over non-cancer cell death) if it triggers cell death of one type of cell to a greater extent than the other. A conjugate (e.g., PAC) described herein “selectively” triggers cancer cell death if it triggers cancer cell death to a greater extent than non-cancer cell death. A peptide described herein is “selectively” cytotoxic to cancer cells over non-cancer cells if it is toxic (e.g., by lysing, killing, promoting apoptosis of, or otherwise damaging) to cancer cells to a greater extent than the non-cancer cells. In certain embodiments, the selectivity in any of the foregoing embodiments is at least 1.1-fold, at least 1.5-fold, 2-fold, at least 3-fold, at least 5-fold, at least 10-fold, at least 30-fold, at least 50-fold, at least 100-fold, at least 300-fold, at least 500-fold, at least 1,000-fold, at least 3,000-fold, at least 5,000-fold, at least 10,000-fold, at least 30,000-fold, at least 50,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

### Examples

### General Methods

Solid phase peptide synthesis: Fmoc-based solid-phase peptide synthesis was used to synthesize the promoter peptides and their stapled derivatives. To achieve the i+4 staple lengths, α-methyl, α-alkenyl amino acids were used flanking three residues. For the stapling reaction, Grubbs 1st generation ruthenium catalyst dissolved in dichloroethane was added to the peptides while still on resin. To ensure maximal conversion, three to five rounds of stapling were performed. Once stapled, the appropriate maleimide linker was coupled to the N-terminus and the linker-peptides were cleaved off the resin using trifluoroacetic acid, then precipitated using a hexane:ether (1:1) mixture, and afterwards purified using a prep HPLC. Final linker-peptide characterization for purity was assessed using a UHPLC/MS system.

Cell culture: Cell lines were maintained in appropriate medium supplemented with fetal bovine serum to a final concentration of 10%.

72 or 144 hour cytotoxicity assay: Cells were plated in a 96-well format, and after 24 hour incubation, serial dilutions of PACs from a 1 mg/mL stock, or vehicle, were then added to the cells in a final volume of 100 μl. After incubating at 37° C. for 72 or 144 hours, 100 μl of CellTiter-Glo® reagent was added to the cells, and the plates were incubated 15 minutes at room temperature. Luminescence was then measured on a microplate reader.

Peptide conjugation protocol: (1) Prepare stapled peptide using solid-phase synthesis; (2) On resin, to an N-terminally deprotected stapled peptide, add a linking reagent. For example, 6-maleimidohexanoyl-Val-Cit-p-aminobenzoylcarbonate-4-nitrophenyl ester (Mc-Val-Cit-PABC-PNP) can be used; (3) Cleave from resin and purify by HPLC; (4) In a separate batch, reduce the antibody using tris(2-carboxyethyl)phosphine (TCEP) (see protocol below); (5) Conjugate the stapled peptide to the antibody; (6) Quench unreacted peptide using N-acetylcysteine; and (7) Purify by salt exchange.

Antibody conjugation protocol: (1) Buffer exchange the antibody into PBS-E, pH 6-8, by the following protocol: (a) Dilute a solution of antibody provided by the manufacturer to 1 mg/mL in PBS-E, pH 6-8, (b) Remove the loading buffer by centrifugating using a spin column. Add more PBS-E and equilibrate by centrifugating further, (c) Add antibody solution (1 mg/mL), centrifugate, collecting the flowthrough, (d) Measure the antibody concentration in the eluate, and (e) Use an ultrafiltration device per manufacturer instructions to raise antibody concentration to 10 mg/mL; (2) Reduce antibody with TCEP; (3) Conjugate cytotoxin/peptide through co-incubation at room temperature; (4) Quench reaction with n-acetylcysteine; and (5) Purify.

Antibody peptide/cytotoxin co-conjugation protocol: (1) Buffer exchange the antibody into PBS-E, pH 6-8, by the following protocol: (a) Dilute a solution of antibody provided by the manufacturer to 1 mg/mL in PBS-E, pH 6-8, (b) Remove the loading buffer by centrifugating using a spin column. Add more PBS-E and equilibrate by centrifugating further, (c) Add antibody solution (1 mg/mL), centrifugate, collecting the flowthrough, (d) Measure the antibody concentration in the eluate, and (e) Use an ultrafiltration device per manufacturer instructions to raise antibody concentration to 10 mg/mL; (2) Reduce antibody with TCEP; (3) Conjugate cytotoxin/peptide through co-incubation at room temperature; (4) Reduce conjugate further with DTT at room temperature; (5) Purify by multiple rounds of ultrafiltration to afford reduced mono-conjugate at 10 mg/ml in PBS-E; (6) Conjugate cytotoxin/peptide at 25° C.; (7) and purify.

Antibody SMCC peptide conjugation protocol: (1) Buffer exchange the antibody into PBS-E, pH 6-8, by the following protocol: (a) Dilute a solution of antibody provided by the manufacturer to 1 mg/mL in PBS-E, pH 6-8, (b) Remove the loading buffer by centrifugating using a spin column. Add more PBS-E and equilibrate by centrifugating further, (c) Add antibody solution (1 mg/mL), centrifugate, collecting the flowthrough, (d) Measure the antibody concentration in the eluate, and (e) Use an ultrafiltration device per manufacturer instructions to raise antibody concentration to 10 mg/mL; (2) Conjugate with Succinimidyl-4-(N-maleimidomethyl)cyclohexane-1-carboxylate (SMCC) through co-incubation at room temperature; (3) Purify; (4) Conjugate with thiol-containing peptide; (5) and purify. If further conjugation is needed to make a co-conjugate, (6) add DTT for reduction at 25° C.; (7) Purify into PBS-E; (7) Conjugate second payload at 25° C.; (8) and purify.

EGFR Degradation In Vitro Assay: Cells were plated in a 24-well format, and after 24-hour incubation, PACs and their relevant unconjugated antibody at a final concentration of 10 μg/mL, or vehicle, were then added to the cells in a final volume of 500 μL in the presence or absence of cycloheximide at 400 μg/mL. After incubating at 37° C. for 24 hours, media was removed, and ice-cold RIPA buffer was added to the cells to lyse them. Lysates were then centrifuged, and supernatant collected for western blot analysis of EGFR protein levels. GAPDH protein levels were used as a loading control.

Antibody uptake analysis by flow cytometry: Cells that express the target of interest were first rinsed briefly with accutase, then were incubated with accutase until detached. Once detached, cells were spun down and resuspended in media and plated in a 96-well plate format. Cells were then treated with PACs and relevant parent antibody at 10 nM for one hour on ice and then washed twice. One plate was taken immediately for Fc staining to measure antibody surface levels at time zero. Other plates were incubated for various time lengths (Up to 4 hours) at 37° C. in buffer and then processed via Fc staining with a fluorescent anti-human IgG Fc specific antibody. Cells were then analyzed via flow cytometry to determine relative antibody surface levels compared to the time zero plate.

### Protein Internalization Examples

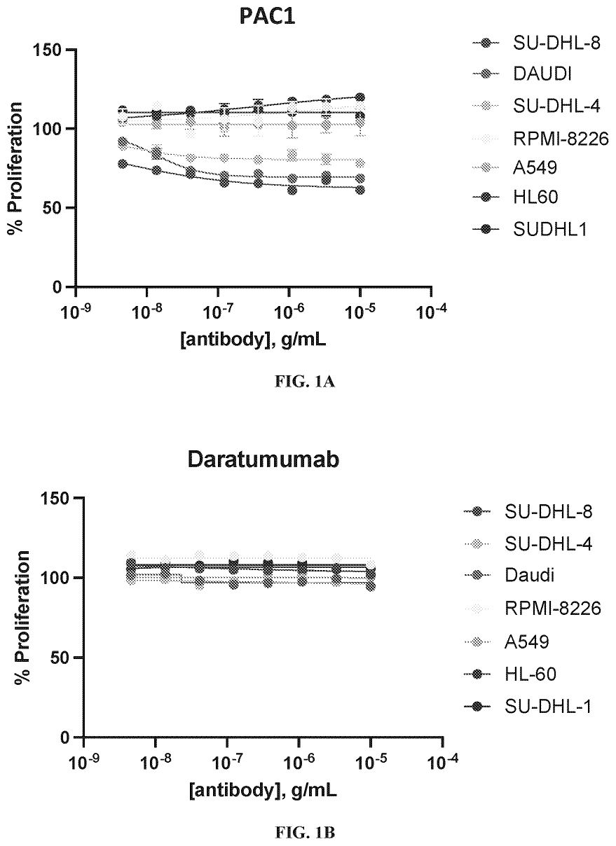

FIG. 1A shows anti-proliferative activity of PAC1 in various cell lines. FIG. 1B shows anti-proliferative activity of daratumumab (not conjugated to a promoter peptide) in various cell lines.

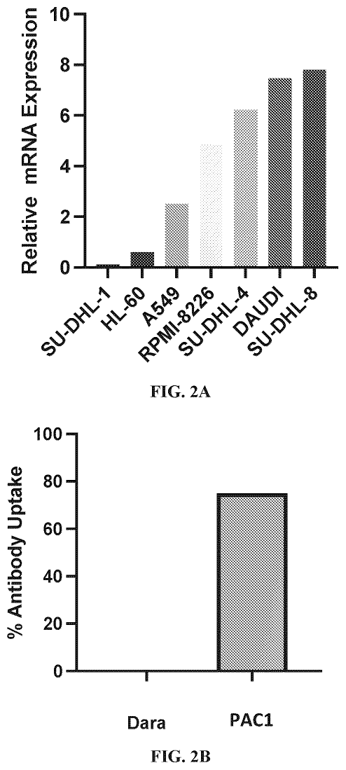

FIG. 2A shows CD38 RNA expression in various cell lines derived from DepMap database. FIG. 2B shows anti-CD38 antibody (daratumumab; “Dara”) uptake in CD38+ cell line SUDHL8; unconjugated daratumumab compared to PAC1.

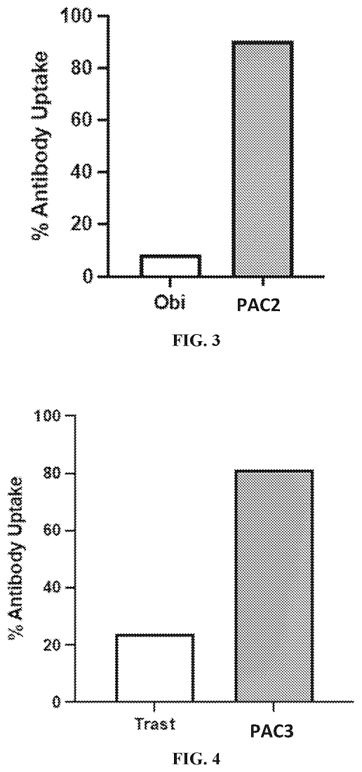

FIG. 3 shows anti-CD20 antibody (obinutuzumab; “Obi”) uptake in a CD20+ cell line SUDHL4; unconjugated obinutuzumab compared to PAC2.

FIG. 4 shows anti-HER2 antibody (trastuzumab; “Trast”) uptake in a HER2+ cell line SKOV3; unconjugated trastuzumab compared to PAC3.

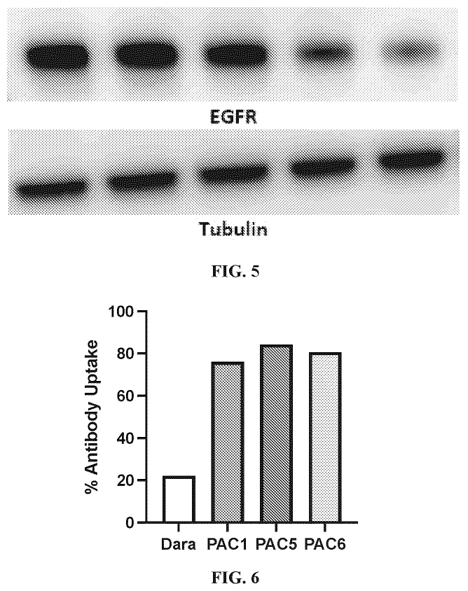

FIG. 5 shows time course of degradation of EGFR in an EGFR+ cell line (COLO678) with PAC4. Lanes from left to right: Time 0, 1 hour post-treatment, 4 hours post-treatment, 8 hours post-treatment, 24 hours post-treatment.

FIG. 6 shows anti-CD38 antibody (daratumumab; “Dara”) uptake in CD38+ cell line SUDHL4; unconjugated daratumumab compared to PAC1, PAC5 and PAC6.

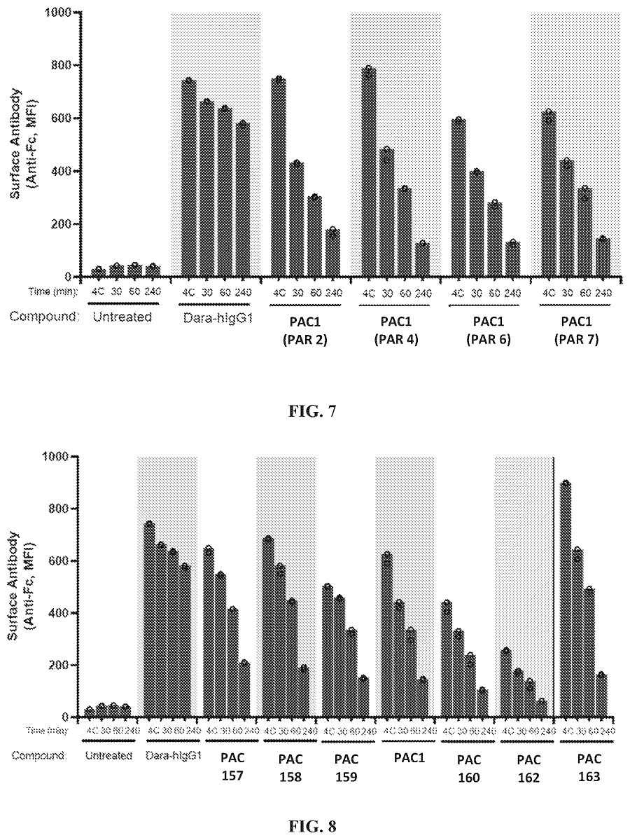

FIG. 7 shows antibody internalization with different peptide-antibody ratios overtime, and FIG. 8 shows antibody internalization with different linkers over time. PAR=peptide-antibody ratio.

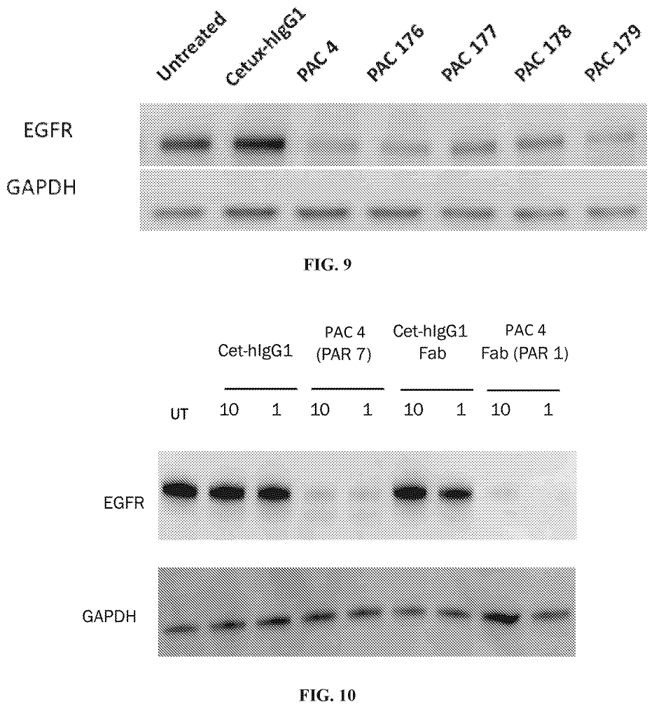

FIG. 9 shows EGFR degradation with various stapled promoter peptides conjugated to Cetuximab after 24 hours of treatment. FIG. 10 shows EGFR degradation with full antibody and Fab fragment conjugated to a promoter peptide after 24 hours of treatment in the presence of cycloheximide. PAR=peptide-antibody ratio.

### Cellular Proliferation Assays

Table 9A below shows the antiproliferative activity of PACs described herein comprising the anti-CD38 mAb daratumumab. Activity was tested in DAUDI and SUDHL4 cell lines.

TABLE 9A
Antiproliferative Activity of PACs Comprising Daratumumab

| PAC# | DAUDI (IC50; ng/mL) | SUDHL4 (IC50; ng/mL) |
|---|---|---|
| 11 | 8 | >10,000 |
| 12 | 41 | 21 |
| 13 | 55 | 65 |
| 14 | 16 | >10,000 |
| 15 | >10,000 | >10,000 |
| 16 | 39 | >10,000 |
| 17 | 44 | >10,000 |
| 18 | 34 | 3 |
| 19 | 12 | <4 ng/mL |
| 20 | 17 | <4 ng/mL |
| 21 | >10,000 | >10,000 |
| 22 | >10,000 | >10,000 |
| 23 | >10,000 | >10,000 |
| 24 | 38 | 32 |
| 25 | 65 | >10,000 |
| 26 | 120 | >10,000 |
| 27 | >10,000 | >10,000 |
| 28 | 24 | <4 ng/mL |
| 29 | 23 | <4 ng/mL |
| 30 | 36 | 19 |
| 31 | 43 | 23 |
| 32 | 10 | <4 ng/mL |
| 33 | 18 | 11 |
| 34 | 110 | >10,000 |
| 35 | 16 | <4 ng/mL |
| 36 | 63 | <4 ng/mL |
| 37 | 40 | 18 |
| 38 | >10,000 | >10,000 |
| 39 | 94 | >10,000 |
| 40 | 13 | >10,000 |
| (A1)* | 11 | <4 ng/mL |
| (A2)* | 21 | 9 |
| 41 | 7 | 6 |
| 42 | 29 | 6 |
| 43 | 45 | 7 |
| 44 | 68 | 14 |
| 45 | 38 | 7 |
| 56 | <4 ng/mL | N/A |
| 57 | 8 | N/A |
| 58 | 6 | <4 ng/mL |
| 59 | <4 ng/mL | N/A |
| 60 | <4 ng/mL | N/A |
| 61 | 6 | <4 ng/mL |
| (A6)* | 50 | 4 |
| 62 | 9 | <4 ng/mL |
| 63 | <4 ng/mL | <4 ng/mL |
| 64 | 6 | <4 ng/mL |
| 65 | 13 | N/A |
| 66 | 7 | <4 ng/mL |
| 67 | 12 | <4 ng/mL |
| (A8)* | 17 | 11 |
| 68 | <4 ng/mL | <4 ng/mL |
| 69 | <4 ng/mL | <4 ng/mL |
| 70 | <4 ng/mL | <4 ng/mL |
| 71 | 8 | N/A |
| 72 | <4 ng/mL | N/A |
| 73 | <4 ng/mL | <4 ng/mL |
| (A9)* | 5 | <4 ng/mL |
| 74 | 11 | <4 ng/mL |
| 75 | 15 | <4 ng/mL |
| 76 | 7 | <4 ng/mL |
| 77 | 13 | <4 ng/mL |
| 78 | 10 | <4 ng/mL |
| 79 | 12 | <4 ng/mL |
| 95 | 22 | <4 ng/mL |
| 96 | 34 | 10 |
| 97 | <4 ng/mL | <4 ng/mL |
| 98 | 12 | <4 ng/mL |
| 99 | 6 | <4 ng/mL |
| 100 | 8 | <4 ng/mL |
| 101 | 10 | <4 ng/mL |
| 102 | 9 | <4 ng/mL |
| 103 | 6 | <4 ng/mL |
| 104 | 15 | <4 ng/mL |
| 105 | <4 ng/mL | <4 ng/mL |
| 106 | 4 | <4 ng/mL |
| 107 | <4 ng/mL | <4 ng/mL |
| 108 | 6 | <4 ng/mL |
| 109 | 11 | 4 |
| 110 | N/A | N/A |
| 111 | N/A | N/A |
| 112 | N/A | N/A |
| 113 | N/A | N/A |
| 114 | N/A | N/A |
| 115 | N/A | N/A |
| 116 | <4 ng/mL | <4 ng/mL |
| 117 | <4 ng/mL | 5 |
| 118 | <4 ng/mL | <4 ng/mL |
| 119 | 6 | 5 |
| 120 | <4 ng/mL | <4 ng/mL |
| 121 | 5 | <4 ng/mL |
| 122 | 19 | 6 |
| 123 | 30 | 7 |
| 124 | 16 | <4 ng/mL |
| 125 | 5 | <4 ng/mL |
| 126 | <4 ng/mL | <4 ng/mL |
| 127 | 6 | <4 ng/mL |
| 128 | 5 | <4 ng/mL |
| 129 | 5 | <4 ng/mL |
| 130 | 8 | <4 ng/mL |
| 131 | 8 | <4 ng/mL |
| 132 | 10 | <4 ng/mL |
| 133 | 9 | <4 ng/mL |
| 134 | 12 | 4 |
| 135 | 10 | <4 ng/mL |
| 136 | 9 | <4 ng/mL |
| 137 | 13 | <4 ng/mL |
| 138 | 13 | <4 ng/mL |
| 139 | 17 | <4 ng/mL |
| 140 | 17 | <4 ng/mL |
| 141 | 20 | <4 ng/mL |
| 142 | 19 | <4 ng/mL |
| 143 | 17 | <4 ng/mL |
| (A12)* | 100 | 258 |
| 144 | <4 ng/mL | <4 ng/mL |
| 145 | 14 | <4 ng/mL |
| (A13)* | 750 | >10,000 |
| (A14)* | 16 | N/A |
| (A15)* | 270 | 297 |
| (A16)* | 137 | 466 |
| (A17)* | 1511 | 1195 |
| 164 | 15 | 15 |
| 165 | N/A | N/A |
| 166 | 35 | 90 |
| (A18)* | 77 | 94 |
| 167 | 39 | N/A |

*(A#) indicates conjugates provided in Table 10 below that do not comprise promoter peptides.

Table 9B below shows the antiproliferative activity of PACs described herein comprising the anti-HER3 mAb patritumab. Activity was tested in a SKBR3 cell line.

TABLE 9B
Antiproliferative Activity of PACs Comprising Patritumab

| PAC# | SKBR3 (IC50; ng/mL) |
|---|---|
| (A3)* | N/A |
| 46 | N/A |
| 47 | N/A |
| 48 | N/A |
| 49 | N/A |
| 50 | N/A |
| (A4)* | 412 |
| 51 | 24 |
| (A10)* | 6 |
| 80 | 4 |
| 82 | 13 |
| 83 | 256 |

*(A#) indicates conjugates provided in Table 10 below that do not comprise promoter peptides.

Table 9C below shows the antiproliferative activity of PACs described herein comprising the anti-Folate Receptor Alpha mAb mirvetuximab. Activity was tested in a JEG3 cell line.

TABLE 9C
Antiproliferative Activity of PACs Comprising Mirvetuximab

| PAC# | JEG3 (IC50; ng/mL) |
|---|---|
| 52 | N/A |
| 53 | N/A |
| 54 | N/A |
| (A5)* | N/A |
| 55 | N/A |
| (A11)* | 19 |
| 81 | 8 |
| 84 | N/A |
| 85 | N/A |
| 86 | N/A |
| 87 | N/A |
| 88 | N/A |
| 89 | 7 |
| 90 | 7 |
| 91 | 9 |
| 92 | 13 |
| 93 | 8 |
| 94 | 10 |

*(A#) indicates conjugates provided in Table 10 below that do not comprise promoter peptides.

Tables 9D-9G below show antiproliferative activity of PACs described herein comprising anti-EGFR, CD33, CD19, and CD20 antibodies, respectively.

TABLE 9D
Antiproliferative Activity of PACs Comprising Nimotuzumab

| PAC# | NCI-H292 (IC50; ng/mL) | SKOV3 (IC50; ng/mL) |
|---|---|---|
| 168 | 8 | >10,000 |
| 169 | 41 | 21 |

TABLE 9E
Antiproliferative Activity of PACs Comprising Vadastuximab

| PAC# | MV-411 (IC50; ng/mL) |
|---|---|
| 170 | 8 |
| (A19)* | 41 |

*(A#) indicates conjugates provided in Table 10 below that do not comprise promoter peptides.

TABLE 9F
Antiproliferative Activity of PACs Comprising
Inebilizumab or Tafasitamab

| PAC# | SU-DHL-4 (IC50; ng/mL) | SU-DHL-8 (IC50; ng/mL) |
|---|---|---|
| 171 | <1 | <1 |
| 173 | <1 | <1 |

TABLE 9G
Antiproliferative Activity of PACs Comprising Obinutuzumab

| PAC# | SU-DHL-4 (IC50; ng/mL) | DAUDI (IC50; ng/mL) |
|---|---|---|
| 172 | <1 | <1 |

Conjugates A1-All referenced in Tables 9A-9C above are provided below in Table 10. Conjugates in Table 10 do not comprise promoter peptides and are provided for comparison purposes, for example.

TABLE 10
ADC Reference Conjugates

| ADC# | Antibody | Cytotoxin | Linker-Cytotoxin | Target |
|---|---|---|---|---|
| A1 | Daratumumab | MMAE | T2 | CD38 |
| A2 | Daratumumab | MMAF | T1* | CD38 |
| A3 | Patritumab | MMAF | T1* | HER3 |
| A4 | Patritumab | MMAE | T2 | HER3 |
| A5 | Mirvetuximab | MMAE | T2 | Folate Receptor Alpha |
| A6 | Daratumumab | MMAE | T4 | CD38 |
| A7 | Daratumumab | MMAE | T4* | CD38 |
| A8 | Daratumumab | MMAF | T3 | CD38 |
| A9 | Daratumumab | MMAF | T1 | CD38 |
| A10 | Patritumab | MMAE | T4 | HER3 |
| A11 | Mirvetuximab | MMAE | T4 | Folate Receptor Alpha |
| A12 | Daratumumab | DM1 | T5 | CD38 |
| A13 | Daratumumab | Deruxtecan | T10 | CD38 |
| A14 | Daratumumab | Exatecan | T12 | CD38 |
| A15 | Daratumumab | Exatecan | T6 | CD38 |
| A16 | Daratumumab | Exatecan | T8 | CD38 |
| A17 | Daratumumab | Exatecan | T7 | CD38 |
| A18 | Daratumumab | MIK665 | T9 | CD38 |
| A19 | Vadastuximab** | Deruxtecan | T10 | CD33 |

*Linker-Cytotoxin is conjugated to a lysine of the antibody via an AJICAP reagent. See, e.g., Yamada et al. “AJICAP: Affinity Peptide Mediated Regiodivergent Functionalization of Native Antibodies” Angew. Chem. Int. Ed. 2019, vol. 58, 5592-5597.
**Vadastuximab S239C.

### EQUIVALENTS AND SCOPE

In the claims, articles such as “a,” “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of promoting uptake of an agent into a cell, the method comprising contacting the cell with a conjugate, wherein the conjugate comprises a promoter peptide conjugated to the agent; and wherein the promoter peptide is a stapled peptide.

2. A method of promoting uptake of an agent into a cell, the method comprising contacting the cell with a conjugate, wherein the conjugate comprises a promoter peptide conjugated to the agent; and wherein the promoter peptide is a Magainin peptide.

3. A method of promoting uptake of an agent into a cell, the method comprising contacting the cell with a conjugate, wherein the conjugate comprises a promoter peptide conjugated to the agent; and wherein the promoter peptide comprises any one of SEQ ID NOs: 5-19.

4. The method of any one of claims 1-3, wherein the percent uptake of the agent into the cell is greater than 25%.

5. The method of any one of claims 1-3, wherein the percent uptake of the agent into the cell is greater than 50%.

6. The method of any one of claims 1-3, wherein the percent uptake of the agent into the cell is greater than 75%.

7. The method of any one of claim 1-6, wherein uptake of the agent into the cell is increased by at least 25%, relative to uptake of the agent not conjugated to the promoter peptide.

8. The method of any one of claim 1-6, wherein uptake of the agent into the cell is increased by at least 100%, relative to uptake of the agent not conjugated to the promoter peptide.

9. The method of any one of claim 1-6, wherein uptake of the agent into the cell is increased by at least 400%, relative to uptake of the agent not conjugated to the promoter peptide.

10. The method of any one of claims 1-6, wherein uptake of the agent into the cell is increased by at least 800%, relative to uptake the agent not conjugated to the promoter peptide.

11. The method of any one of claims 1-10, wherein the agent is a protein.

12. The method of any one of claims 1-11, wherein the agent is an antibody or antigen-binding fragment thereof.

13. The method of claim 12, wherein the antibody or antigen-binding fragment thereof is an antibody-drug conjugate (ADC).

14. The method of claim 13, wherein the ADC comprises the antibody or antigen-binding fragment thereof conjugated to a small molecule cytotoxic agent.

15. A method of promoting uptake of a small molecule into a cell, the method comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a stapled peptide.

16. A method of promoting uptake of a small molecule into a cell, the method comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide is a Magainin peptide.

17. A method of promoting uptake of a small molecule into a cell, the method comprising contacting the cell with a peptide-antibody conjugate (PAC), wherein the PAC comprises a promoter peptide conjugated to an antibody-drug conjugate (ADC); wherein ADC comprises the small molecule conjugated to an antibody or antigen-binding fragment thereof; and wherein the promoter peptide comprises any one of SEQ ID NOs: 5-19.

18. The method of any one of claims 15-17, wherein the promoter peptide is conjugated to the antibody or antigen-binding fragment thereof of the ADC.

19. The method of any one of claims 15-18, wherein the percent uptake of the small molecule into the cell is greater than 25%, greater than 50%, or greater than 75%.

20. The method of any one of claims 15-19, wherein uptake of the small molecule into the cell is increased by at least 25%, by at least 100%, by at least 400%, or by at least 800%, relative to uptake of the small molecule when the ADC is not conjugated to the promoter peptide.

21. The method of any one of claims 15-20, wherein the small molecule is a small molecule cytotoxic agent.

22. A method of promoting uptake of a target protein into a cell, the method comprising contacting the cell with a conjugate, wherein the conjugate comprises a promoter peptide conjugated to an agent that binds the target protein; and wherein the promoter peptide is a stapled peptide.

23. A method of promoting uptake of a target protein into a cell, the method comprising contacting the cell with a conjugate, wherein the conjugate comprises a promoter peptide conjugated to an agent that binds the target protein; and wherein the promoter peptide is a Magainin peptide.

24. A method of promoting uptake of a target protein into a cell, the method comprising contacting the cell with a conjugate, wherein the conjugate comprises a promoter peptide conjugated to an agent that binds the target protein; and wherein the promoter peptide comprises any one of SEQ ID NOs: 5-19.

25. The method of any one of claims 22-24, wherein the target protein is on the surface of the cell.

26. The method of any one of claims 22-25, wherein the target protein is a receptor or a fragment thereof.

27. The method of any one of claims 22-26, wherein the target protein is selected from CD20, CD38, HER2, HER3, FRα, CD33, CD20, CD19, PD-L1, Nectin-4, TROP-2, Claudin 18.2, c-MET, Tfr1, BCMA, CD70, PSMA, Tissue factor, MUC-16, NaPi2b, Axl, B7-H4, B7-H3, and EGFR.

28. The method of claim any one of claims 22-27, wherein the target protein is a soluble protein.

29. The method of any one of claims 22-28, wherein the target protein is degraded after internalization into the cell.

30. The method of any one of claims 22-29, wherein the agent that binds the target protein is an antibody or an antigen-binding fragment thereof.

31. The method of any one of claims 22-30, wherein the percent uptake of the target protein into the cell is greater than 25%, greater than 50%, or greater than 75%.

32. The method of any one of claims 22-31, wherein uptake of the target protein into the cell is increased by at least 25%, by at least 100%, by at least 400%, or by at least 800%, relative to uptake of the target protein when the agent that binds the target protein is not conjugated to the promoter peptide.

33. The method of any one of the preceding claims, wherein the antibody or antigen-binding fragment thereof is directed to a target protein expressed on a cancer cell.

34. The method of any one of the preceding claims, wherein the antibody is an antibody directed against CD38, or an antigen-binding fragment thereof.

35. The method of any one of the preceding claims, wherein the antibody is daratumumab, isatuximab, or mezagitamab.

36. The method of any one of the preceding claims, wherein the antibody is an antibody directed against CD20, CD33, or CD19, or an antigen-binding fragment thereof.

37. The method of any one of the preceding claims, wherein the antibody is obinutuzumab, vadastuximab, nimotuzumab, inebilizumab, or tafastimab.

38. The method of any one of the preceding claims, wherein the antibody is an antibody directed against HER2, or an antigen-binding fragment thereof.

39. The method of any one of the preceding claims, wherein the antibody is trastuzumab.

40. The method of any one of the preceding claims, wherein the antibody is an antibody directed against EGFR, or an antigen-binding fragment thereof.

41. The method of any one of the preceding claims, wherein the antibody is cetuximab.

42. The method of any one of the preceding claims, wherein the antibody or antigen-binding fragment thereof is a nanobody.

43. The method of any one of the preceding claims, wherein the antibody is a monoclonal antibody (mAb), or an antigen-binding fragment thereof.

44. The method of any one of the preceding claims, wherein the stapled peptide is a singly stapled, doubly stapled, or stitched peptide.

45. The method of any one of the preceding claims, wherein the stapled peptide is a stapled Magainin peptide.

46. The method of any one of the preceding claims, wherein the stapled peptide is a stapled Magainin II peptide.

47. The method of any one of the preceding claims, wherein the Magainin peptide is an unstapled Magainin peptide.

48. The method of any one of the preceding claims, wherein the Magainin peptide is an unstapled Magainin II peptide.

49. The method of any one of the preceding claims, wherein the promoter peptide comprises the amino acid sequence:
(SEQ ID NO: 1)
G X¹ G Dap F X² Dap Dap Dap Dap Dap F G Dap A X³ V
G E X⁴ A Dap Dap,
or a pharmaceutically acceptable salt thereof, wherein:
X¹, X², X³, and X⁴ are each independently a natural or non-natural amino acid;
optionally wherein X¹ and X² are connected via a crosslink, and X³ and X⁴ are connected via a crosslink; and
wherein the amino acid sequence includes 0 to 11 amino acid substitutions, inclusive.

50. The method of claim 49, wherein the amino acid sequence includes 0 to 5 amino acid substitutions, inclusive.

51. The method of claim 49 or 50, wherein the amino acid sequence includes 1 or 2 amino acid substitutions.

52. The method of any one of the preceding claims, wherein the promoter peptide comprises the amino acid sequence:
(SEQ ID NO: 2)
G X¹ G Dap F X² Dap Dap Dap Dap Dap FG Dap A X³ V
G E X⁴ A Dap Dap G,
or a pharmaceutically acceptable salt thereof, wherein:
X¹, X², X³, and X⁴ are each independently a natural or non-natural amino acid;
optionally wherein X¹ and X² are connected via a crosslink, and X³ and X⁴ are connected via a crosslink.

53. The method of any one of claims 49-52, wherein X¹ and X² are connected via a crosslink and X³ and X⁴ are connected via a crosslink.

54. The method of any one of the preceding claims, wherein the promoter peptide is conjugated to the agent, the ADC, or the agent that binds a target protein, via a linker.

55. The method of claim 54, wherein the linker comprises optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

56. The method of claim 54 or 55, wherein the linker is a cleavable linker.

57. The method of claim 56, wherein the cleavable linker is pH cleavable or cleavable by a protease, esterase, or intracellular disulfide reduction.

58. The method of claim 54 or 55, wherein the linker is a non-cleavable linker.

59. The method of any one of claims 54-58, wherein the linker is a peptidic linker.

60. The method of claim 59, wherein the peptidic linker comprises —Y^{A}Y^{B}Y^{C}Y^{D}— (SEQ ID NO: 140), wherein Y^{A} is glycine, glutamic acid, lysine, or is absent; Y^{B} is valine, phenylalanine, alanine, tyrosine, or glycine; Y^{C} is citrulline, arginine, lysine, alanine, or glycine; and Y^{D} is glycine or is absent.

61. The method of claim 59 or 60, wherein the peptidic linker comprises -valine-citrulline-.

62. The method of claim 61, wherein the peptidic linker is any one of L1-L34.

63. The method of any one of the preceding claims, wherein the cell is in a subject, and the method comprises administering to the subject an effective amount of the conjugate or PAC.

64. The method of claim 63, wherein the subject is diagnosed with a disease.

65. The method of claim 64, wherein the disease is cancer or a tumor.

66. The method of any one of claims 63-65, wherein the effective amount is a therapeutically effective amount.

67. A peptide-antibody conjugate (PAC) selected from those in Table 2A, Table 2B, and Table 4.

68. A peptide comprising the amino acid sequence:
(SEQ ID NO: 5)
X⁵X⁶X⁷X⁵X⁶X⁷X⁵,
or a pharmaceutically acceptable salt thereof, wherein:
each X⁵ is independently selected from arginine, histidine, Dap, Dab, Orn, homo-lysine, homo-arginine, homo-histidine, 4-guanidino-phenylalanine, and 4-amino-phenylalanine;
each X⁶ is independently selected from modified or unmodified phenylalanine, leucine, isoleucine, tryptophan, tyrosine, and norleucine; and
each X⁷ is independently selected from alanine, valine, leucine, isoleucine, glycine, and serine.

69. The peptide of claim 68, wherein the peptide comprises the amino acid sequence SEQ ID NO: 6 or SEQ ID NO: 19, or a pharmaceutically acceptable salt thereof.

70. The peptide of claim 68, wherein the peptide comprises any one of SEQ ID NOs: 7-18, or a pharmaceutically acceptable salt thereof.

71. A conjugate comprising a peptide of any one of claims 68-70, or a pharmaceutically acceptable salt thereof, conjugated to an agent.

72. The conjugate of claim 71, wherein the agent is an antibody or an antigen-binding fragment thereof.

73. A peptide-antibody conjugate (PAC) comprising a promoter peptide of any one of SEQ ID NOs: 5-19, or a pharmaceutically acceptable salt thereof, conjugated to an antibody or an antigen-binding fragment thereof.

74. The PAC of claim 73, wherein the promoter peptide is of any one of SEQ ID NOs: 7-18.

75. A peptide-antibody conjugate (PAC) comprising a promoter peptide or a pharmaceutically acceptable salt thereof conjugated to an antibody or an antigen-binding fragment thereof, wherein the promoter peptide comprises any one of SEQ ID NOs: 142-174.

76. The PAC of claim 75, wherein the antibody or antigen-binding fragment thereof is an antibody against CD38, HER3, EGFR, or Folate Receptor Alpha, or an antigen-binding fragment thereof.

77. The PAC of claim 75, wherein the antibody or antigen-binding fragment thereof is daratumumab, patritumab, cetuximab, or mirvetuximab, or an antigen-binding fragment thereof.

78. The PAC of any one of claims 75-77 further comprising a small molecule cytotoxic agent.

79. The PAC of claim 78, wherein the small molecule cytotoxic agent is selected from auristatins, maytansinoids, camptothecins, anthracyclines, tubulysins, calicheamycins, duocarymycins, pyrrolobenzodiazepines (PBD) dimers, pyridinobenzodiazepines (PDDs), amatoxins, and immunotoxins.

80. The PAC of claim 78, wherein the small molecule cytotoxic agent is selected from those in Table 3A and Table 3B.

81. The PAC of claim 78, wherein the small molecule cytotoxic agent is MMAE or MMAF, or a pharmaceutically acceptable salt thereof.

82. A stapled or unstapled peptide comprising any one of SEQ ID NOs: 142-174, or a pharmaceutically acceptable salt thereof.
